(19) United States
(12) Patent Application Publication
(10) Pub. No.: US 2010/0173828 A1
(43) Pub. Date: Jul. 8, 2010

(54) Aß(X - 38 .. 43) oligomers, and processes, compositions, and uses thereof

(75) Inventors: Heinz Hillen, Hassloch (DE); Andreas Striebinger, Speyer (DE); Simone Giaisi, Mannheim (DE); Stefan Barghorn, Mannheim (DE); Gerald Gellermann, Mannheim (DE); Ulrich Ebert, Mannheim (DE); Joerg Moelleken, Heidelberg (DE)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT GmbH & Co. KG, Wiesbaden-Delkenheim (DE)

(21) Appl. No.: 12/509,315

(22) Filed: Jul. 24, 2009

Related U.S. Application Data
(60) Provisional application No. 61/083,597, filed on Jul. 25, 2008.

(51) Int. Cl. A61K 38/17; C07K 14/435; A61P 25/28; C40B 30/04; G01N 33/53

(52) U.S. Cl. 514/8; 530/395; 506/9; 435/7.92

(57) ABSTRACT

The present invention relates to an Aβ(X-38 . . . 43) oligomer having a high molecular weight, or a derivative thereof, a process for preparing the oligomer or derivative, compositions comprising the oligomer or derivative, and uses of the oligomer or derivative such as its use for treating or preventing an amyloidosis (e.g. by active immunization), for diagnosing an amyloidosis, and for providing agents that are capable of binding to the Aβ(X-38 . . . 43) oligomer or derivative. The subject invention also describes agents that are capable of binding to the Aβ(X-38 . . . 43) oligomer or derivative, e.g. antibodies, compositions comprising the agents, and uses of the agents such as their use for treating or preventing an amyloidosis (e.g. by passive immunization) and for diagnosing an amyloidosis.

This application claims priority to the provisional application Ser. No. 61/083,597 filed Jul. 25, 2008, which is incorporated herein by reference.

### FIELD OF THE INVENTION

The subject invention relates to an Aβ(X-38 . . . 43) oligomer or a derivative thereof, a process for preparing the oligomer or derivative, compositions comprising the oligomer or derivative, and uses of the oligomer or derivative such as its use for treating or preventing an amyloidosis (e.g. by active immunization), for diagnosing an amyloidosis, and for providing agents that are capable of binding to the oligomer or derivative. The subject invention also describes agents that are capable of binding to the Aβ(X-38 . . . 43) oligomer or derivative, e.g. antibodies, compositions comprising the agents, and uses of the agents such as their use for treating or preventing an amyloidosis (e.g. by passive immunization) and for diagnosing an amyloidosis.

### BACKGROUND OF THE INVENTION

In 1907, the physician Alois Alzheimer first described the neuropathological features of a form of dementia subsequently named in his honor as Alzheimer's disease (AD). In particular, AD is the most frequent cause for dementia among the aged, with an incidence of about 10% of the population in those above 65 years of age. With increasing age, the probability of disease also rises. Globally, there are about 15 million people affected with the disease and further increases in life expectancy are expected to increase the number of people affected with the disease to about three-fold over the next decades.

From a molecular point of view, Alzheimer's disease (AD) is characterized by a deposit of abnormally aggregated proteins. In the case of extracellular amyloid plaques, these deposits consist mostly of amyloid-β-peptide filaments, and in the case of the intracellular neurofibrillary tangles (NFTs), mostly of the tau protein. The amyloid β(Aβ) peptide arises from the β-amyloid precursor protein by proteolytic cleavage. This cleavage is effected by the cooperative activity of several proteases named α-, β- and γ-secretase. Cleavage leads to a number of specific fragments of differing length. The amyloid plaques consist mostly of peptides with a length of 40 or 42 amino acids (Aβ40, Aβ42). The dominant cleavage product is Aβ40; however, Aβ42 has a much stronger toxic effect. Cerebral amyloid deposits and cognitive impairments very similar to those observed in Alzheimer's disease are also hallmarks of Down's syndrome (trisomy 21), which occurs at a frequency of about 1 in 800 births.

The amyloid cascade hypothesis of Hardy and Higgins postulated that increased production of Aβ(1-42) would lead to the formation of protofibrils and fibrils (i.e., the principal components of Aβ plaques), these fibrils being responsible for the symptoms of Alzheimer's disease. Despite the poor correlation between severity of dementia and Aβ plaque burden deposited, this hypothesis was favored until recently. The discovery of soluble Aβ forms in AD brains, which correlates better with AD symptoms than plaque load does, has, however, led to a revised amyloid-cascade-hypothesis.

Active immunization with Aβ peptides leads to a reduction in the formation as well as to partial dissolution of existing plaques. At the same time, it leads to alleviation of cognitive defects in APP transgenic mouse models.

The results of a phase IIa trial (ELAN Corporation Plc, South San Francisco, Calif., USA and Dublin, UK) of active immunization with AN-1792 (Aβ(1-42) peptide in fibrillary condition of aggregation) suggest that immunotherapy directed to Aβ peptide was successful. In a subgroup of 30 patients, the progression of disease was significantly reduced in patients with positive anti-Aβ antibody titer, measured by MMSE and DAD index.

However, this study was stopped because of serious side effects in the form of a meningoencephalitis (Bennett and Holtzman, 2005, Neurology, 64, 10-12). In particular, meningoencephalitis was characterized by neuroinflammation and infiltration of T-cells into the brain. Presumably, this was due to a T-cell immune response induced by the injection of Aβ(1-42) peptide as antigen.

Accordingly, Aβ monomers, Aβ fibrils and sAPPα may have physiologic function for the body and targeting them may elicit unwanted side effects such as microhemorrhages.

WO 2004/067561 relates to globular oligomers (“globulomers”) of Aβ(1-42) peptide and a process for preparing them. The data suggest the existence of an amyloid fibril independent pathway of Aβ folding and assembly into Aβ oligomers which display one or more unique epitopes (hereinafter referred to as the globulomer epitopes). Since globulomer epitopes were detected in the brain of AD patients and APP transgenic mice and the globulomer specifically binds to neurons and blocks LTP, the globulomer represents a pathologically relevant Aβ conformer. WO 2004/067561 further describes that limited proteolysis of the globulomers yields truncated versions of said globulomers such as Aβ(20-42) or Aβ(12-42) globulomers.

WO 2006/094724 relates to non-diffusible globular Aβ(X-38 . . . 43) oligomers wherein X is selected from the group consisting of numbers 1 . . . 24. These globulomers are said to be obtainable by same processes as described in WO 2004/067561, i.e. SDS- or fatty acid-induced oligomerization of Aβ(1-38 . . . 43) peptide to generate Aβ(1-38 . . . 43) globulomers, and limited proteolysis of Aβ(1-38 . . . 43) globulomers to generate truncated versions thereof, i.e., (Aβ(X-38 . . . 43) globulomers wherein X is selected from the group consisting of numbers 2 . . . 24).

Aβ(20-42) globulomer obtained by proteolytic cleavage has been used to generate globulomer-specific antibodies. For instance, WO 2007/062852 describes several monoclonal antibodies which specifically recognize Aβ(20-42) globulomer.

WO 2006/094724 also describes that truncated Aβ peptides may be subjected to the conditions of SDS- or fatty acid-induced oligomerization. For instance, when Aβ(12-42) peptide was subjected to the conditions of SDS-induced oligomerization a Aβ(12-42) globulomer preparation was obtained which gave rise to a 12 kDa band in SDS-PAGE (see example 6 of WO 2006/094724). This Aβ(12-42) globulomer could successfully be used for generating monoclonal antibodies that specifically recognize Aβ globulomers.

However, when an Aβ(20-42) peptide was subjected to the conditions of SDS- or fatty acid-induced oligomerization a preparation was obtained which gave rise to a 32 kDa band in SDS-PAGE but contained only very small amounts of globulomer (see example 5 of WO 2006/094724).

It was therefore an object of the present invention to provide oligomers of Aβ peptide which are capable of eliciting a selective immune response for Aβ oligomers. To this end, it was a further object of the present invention to provide Aβ oligomers which do not comprise N-terminal sequences of Aβ peptide.

### SUMMARY OF THE INVENTION

The present invention provides high molecular weight Aβ(X-38 . . . 43) oligomers which are directly obtainable from Aβ(X-38 . . . 43) peptide, wherein X is 12 . . . 24, derivatives thereof.

Particular embodiments of said oligomers or derivatives include:

the oligomers or derivatives, wherein X is 15;
the oligomers or derivatives, wherein X is 18;
the oligomers or derivatives, wherein X is 19;
the oligomers or derivatives, wherein X is 20;
the oligomers or derivatives, wherein X is 21;
the oligomers or derivatives, wherein X is 22;
the oligomers or derivatives of any one of the preceding embodiments, wherein Aβ(X-38 . . . 43) is Aβ(X-42);

The oligomers or derivatives of any one of the preceding embodiments, wherein Aβ(X-38 . . . 43) is Aβ(X-40);

the oligomers or derivatives, wherein Aβ(X-38 . . . 43) is Aβ(20-42);
the oligomers or derivatives of any one of the preceding embodiments, wherein the oligomers have an apparent molecular weight in size exclusion chromatography (1.0% SDS) of at least 200 kDa, at least 300 kDa, at least 400 kDa, or at least 500 kDa;
the oligomers or derivatives of the preceding embodiments, wherein size exclusion chromatography is carried out on a Superose 6 HR 10/300 GL column using as mobile phase a buffer composed of 20 mM NaH₂PO₄, 140 mM NaCl, pH 7.4 and 1% SDS with a flow-rate of 0.5 ml/min;
the oligomers or derivatives of any one of the preceding embodiments, wherein the oligomers are soluble;
the oligomers or derivatives of any one of the preceding embodiments, wherein the oligomer has a size in the range of about 5 nm to about 500 nm, when determined by photon correlation spectroscopy;
the oligomers or derivatives of any one of the preceding embodiments, wherein the oligomers are reactive with a monoclonal antibody selected from the group consisting of 7C6, 7E5, 4D10 and 5F7;
the oligomers or derivatives of any one of the preceding embodiments, wherein the oligomers are capable of eliciting a polyclonal antiserum which comprises an antibody having a binding affinity to the oligomer or derivative or to an Aβ(20-42) truncated globulomer that is greater than the binding affinity of the antibody to at least one Aβ form selected from the group consisting of monomeric Aβ(1-42), monomeric Aβ(1-40), monomeric Aβ(20-42), fibrillomeric Aβ(1-42), fibrillomeric Aβ(1-40) and Aβ(1-42) globulomer;
the oligomers or derivatives of any one of the preceding embodiments, wherein the oligomers are capable of eliciting a polyclonal antiserum having an affinity to the oligomer or derivative or to an Aβ(20-42) truncated globulomer which is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antiserum to at least one Aβ form selected from the group consisting of monomeric Aβ(1-42), monomeric Aβ(1-40), monomeric Aβ(20-42), fibrillomeric Aβ(1-42), fibrillomeric Aβ(1-40) and Aβ(1-42) globulomer;

The present invention also relates to a process for preparing an Aβ(X-38 . . . 43) oligomer or a derivative thereof, wherein X is selected from the group consisting of the numbers 12 . . . 24, which process comprises
(i) dissolving Aβ(X-38 . . . 43) peptide or a derivative thereof in a hydrogen bond-breaking agent;
(ii) adding an amphipatic agent to the solution of Aβ(X-38 . . . 43) peptide or derivative in the hydrogen bond-breaking agent and incubating at a temperature of more than 15° C.

The present invention further relates to a process for preparing an Aβ(X-38 . . . 43) oligomer or a derivative thereof, wherein X is selected from the group consisting of the numbers 12 . . . 24, which process comprises
(i) dissolving Aβ(X-38 . . . 43) peptide or a derivative thereof in a hydrogen bond-breaking agent;
(ii) and incubating the dissolved Aβ(X-38 . . . 43) peptide or derivative at a pH in the range of 8 to 10 and a temperature of more than 15° C.

Particular embodiments of said processes include:

the processes, wherein the solvent is a hydrogen bond-breaking agent;
the processeses of the preceding embodiments, wherein the Aβ(X-38 . . . 43) peptide is a synthetic Aβ(X-38 . . . 43) peptide;
the processes of any one of the preceding embodiments, wherein the hydrogen bond-breaking agent is volatile;
the processes of any one of the preceding embodiments, wherein the hydrogen bond-breaking agent is HFIP;
the processes of any one of the preceding embodiments, wherein the hydrogen bond-breaking agent is an aqueous solution of sodium hydroxide;
the processes of the preceding embodiment, wherein the aqueous solution comprises from 0.025% by weight to 0.25% by weight of sodium hydroxide;
the processes of any one of the preceding embodiments, wherein dissolving the Aβ(X-38 . . . 43) peptide in the hydrogen bond-breaking agent comprises shaking for 15 minutes to 2 hours at 22 to 37° C.;
the processes of any one of the preceding embodiments, wherein the concentration of the Aβ(X-38 . . . 43) peptide dissolved in the hydrogen bond-breaking agent is 2 mg/mL to 50 mg/mL;
the processes of any one of the preceding embodiments, wherein a clear solution is obtained by dissolving the Aβ(X-38 . . . 43) peptide in the hydrogen bond-breaking agent;
the processes of any one of the preceding embodiments, wherein the amphipatic agent is SDS, lauric acid, N-lauroylsarcosin, tert-octylphenol x 9-10EO, nonylphenol x 20EO, 3-(3-cholamidopropyldimethylammonio-1-propane sulfonate, dodecyl-N,N-dimethyl-3-amino-1-propane sulfonate, dodecylamine or a polyethoxylated sorbitol ester;
the processes of any one of the preceding embodiments, wherein the amphipatic agent is added in the form of an aqueous solution;
the processes of the preceding embodiment, wherein the amount of aqueous solution added is 5 to 50 times the volume of the solution of the Aβ(X-38 . . . 43) peptide in the hydrogen bond-breaking agent;
the processes of the preceding embodiments, wherein the aqueous solution comprises 0.05% by weight to 0.7% by weight of amphipatic agent;
the processes of any one of the preceding embodiments, wherein the aqueous solution is buffered and has a pH value of 6.0 to 10.0;
the processes of any one of the preceding embodiments, wherein the buffered aqueous solution comprises 5 mM NaH₂PO₄ and 35 mM NaCl;
the processes of any one of the preceding embodiments, wherein the solution containing the Aβ(X-38 . . . 43) peptide and the buffered aqueous solution are mixed;
the processes of the preceding embodiment, wherein mixing comprises vortexing;
the processes of the preceding embodiments, wherein the pH of the mixture is adjusted to 6.0 to 10.0;
the processes of the preceding embodiments, wherein the pH of the mixture is adjusted to 8.0 to 10.0;
the processes of the preceding embodiment, wherein the adjustment of pH comprises adding an aqueous solution of HCl;
the processes of any one of the preceding embodiments, wherein the mixture is pre-incubated for 2 to 30 minutes;
the processes of the preceding embodiment, wherein pre-incubation comprises shaking the mixture;
the processes of the preceding embodiments, wherein the temperature of pre-incubation is 20 to 37° C.;
the processes of any one of the preceding embodiments, wherein the pre-incubated mixture is centrifuged and supernatant of the centrifuged mixture is obtained;
the processes of any one of the preceding embodiments, wherein the time of incubation is 5 minutes to 30 hours;
the processes of any one of the preceding embodiments, wherein the temperature of incubation is 15 to 50° C.;
the processes of any one of the preceding embodiments, wherein incubation comprises shaking;
the processes of the preceding embodiment, wherein shaking is performed in an orbital shaker;
the processes of any one of the preceding embodiments, wherein incubation comprises incubating at a first temperature from 15 to 50° C. followed by incubation at a second temperature from 15 to 50° C., wherein the second temperature is inferior to the first temperature;
the processes of the preceding embodiment, wherein the time of incubation at the first temperature is 10 minutes to 10 hours and the time of incubation at the second temperature is 20 minutes to 30 hours, respectively;
the processes of any one of the preceding embodiments, wherein the time of incubation at 15 to 50° C. is 5 minutes to 30 hours;
the processes of any one of the preceding embodiments, wherein at least a part of the hydrogen bond-breaking agent is allowed to evaporate;
the processes of the preceding embodiment, wherein the temperature of incubation is 15 to 50° C.;
the processes of the preceding embodiments, wherein the time of incubation is 10 minutes to 10 hours;
the processes of any one of claims 17 to 50, wherein the incubation mixture is centrifuged and the supernatant of the centrifuged incubation mixture is obtained;
the processes of the preceding embodiment, wherein the supernatant of centrifuged incubation mixture is frozen, thawed, again centrifuged and the supernatant of centrifuged mixture is obtained;
the processes of the preceding embodiment, wherein the supernatant of centrifuged incubation mixture is frozen at −5 to −50° C. for 10 minutes to 20 hours;
the processes of any one of the preceding embodiments, which further comprises
(iii) concentrating the incubation mixture;
the processes of the preceding embodiment, wherein the incubation mixture is diluted before concentrated;
the processes of the preceding embodiment, wherein diluting comprises adding a buffered solution;
the processes of the preceding embodiment, wherein the buffered solution has a pH value of 7.4;
the processes of the preceding embodiments, wherein the buffered solution comprises 5 mM NaH₂PO₄ and 35 mM NaCl;
the processes of any one of the preceding embodiments, wherein dilution comprises reducing the concentration of the amphipatic agent below CMC;
the processes of any one of the preceding embodiments, wherein dilution is 2-fold to 20-fold;
the processes of the preceding embodiment, wherein concentrating is done by ultracentifugation;
the processes of the preceding embodiment, wherein the ultracentifugation comprises a 10 to 100 kDa cut-off;
the processes of the preceding embodiments, wherein the ultracentifugation comprises reducing the volume to 1 to 40%;
the processes of any one of the preceding embodiments, wherein the concentrate is centrifuged and the supernatant of the centrifuged concentrate is obtained;
the processes of any one of the preceding embodiments, which further comprises
(iv) reducing salt concentration of the incubation mixture or the concentrated incubation mixture;
the processes of the preceding embodiment, wherein the salt concentration is reduced by subjecting the incubation mixture or the concentrated incubation mixture to dialysis;
the processes of the preceding embodiment, wherein the dialysate is centrifuged and the supernatant of centrifuged dialysate is obtained;
the processes of the preceding embodiment, wherein the salt concentration is reduced by subjecting the incubation mixture or the concentrated incubation mixture to HPLC using a desalting column and an aqueous buffered solution as eluent;
processes for preparing an Aβ(X-38 . . . 43) oligomer or a derivative thereof, wherein X is selected from the group consisting of the numbers 12 . . . 24, which process comprises
(i) dissolving monomeric Aβ(X-38 . . . 43) peptide or a derivative thereof in a hydrogen bond-breaking agent;
(ii) adding an amphipatic agent, mixing, optionally adjusting pH, pre-incubating, centrifuging, obtaining the supernatant, incubating, centrifuging and obtaining the supernatant;
(iii) diluting, concentrating, centrifuging, and obtaining the supernatant; and
(iv) reducing salt concentration, centrifuging and obtaining the supernatant.
   Aβ(X-38 . . . 43) oligomers or derivatives thereof, wherein X is selected from the group consisting of the numbers 12 . . . 24 and which are obtainable by said processes are also included in the invention.

Further, the present invention relates to compositions comprising an Aβ(X-38 . . . 43) oligomer or derivative as defined herein.

Particular embodiments of said compositions include compositions, wherein the composition is a vaccine and further comprises a pharmaceutically acceptable carrier.

The present invention also relates to the use of an Aβ(X-38 . . . 43) oligomer or derivative as defined herein for preparing a pharmaceutical composition for treating or preventing an amyloidosis and to corresponding methods of treating or preventing an amyloidosis in a subject in need thereof, which comprises administering an Aβ(X-38 . . . 43) oligomer or derivative as defined herein to the subject.

Particular embodiments of said use and methods include:

the use and methods, wherein the pharmaceutical composition is for active immunization;
the use and methods of the preceding embodiments, wherein the amyloidosis is Alzheimer's disease;
the use of the preceding embodimetns, wherein the amyloidosis is the amyloidosis of Down's syndrome.

The present invention also relates to the use of an Aβ(X-38 . . . 43) oligomer or derivative as defined herein for preparing a composition for diagnosing an amyloidosis and to corresponding methods of diagnosing an amyloidosis which comprises providing a sample from the subject suspected of having the amyloidosis, contacting the sample with an Aβ(X-38 . . . 43) oligomer or derivative as defined herein for a time and under conditions sufficient for the formation of a complex comprising the Aβ(X-38 . . . 43) oligomer or derivative and an antibody, the presence of the complex indicating the subject has the amyloidosis.

Particular embodiments of said use and methods include:

the use and methods, wherein the amyloidosis is Alzheimer's disease;
the use and methods, wherein the amyloidosis is the amyloidosis of Down's syndrome.

Further, the present invention relates to a method of enriching an agent capable of binding to an Aβ(X-38 . . . 43) oligomer or derivative as defined herein in a preparation comprising said agent, which method comprises the steps of: a) exposing to the Aβ(X-38 . . . 43) oligomer or derivative the preparation comprising the agent for a time and under conditions sufficient for the agent to bind to the Aβ(X-38 . . . 43) oligomer or derivative; and b) obtaining the agent in enriched form.

Particular embodiments of said method include the use, wherein the agent is an antibody, an aptamer or a small molecular weight compound.

Also, the present invention relates to the use of an Aβ(X-38 . . . 43) oligomer or derivative as defined herein for providing an agent that is capable of binding to the Aβ(X-38 . . . 43) oligo-mer or derivative and to corresponding methods, e.g. a method of providing an antibody capable of binding to an Aβ(X-38 . . . 43) oligomer or derivative as defined herein, which comprises
i) providing an antigen comprising the Aβ(X-38 . . . 43) oligomer or derivative;
ii) exposing an antibody repertoire to said antigen; and
iii) selecting from said repertoire an antibody which binds to the Aβ(X-38 . . . 43) oligo-mer or derivative.

Particular embodiments of said use and methods include, wherein the agent is an antibody, a non-antibody binding molecule, an aptamer or a small molecular weight compound.

Antibodies which are obtainable by said process are also described as well as agents capable of binding to an Aβ(X-38 . . . 43) oligomer or derivative of the invention.

Further, the present invention describes compositions comprising an agent capable of binding to an Aβ(X-38 . . . 43) oligomer or derivative of the invention; the use of an agent capable of binding to an Aβ(X-38 . . . 43) oligomer or derivative of the invention for preparing a pharmaceutical composition for treating or preventing an amyloidosis and corresponding methods of treating or preventing an amyloidosis in a subject in need thereof, which comprises administering an agent capable of binding to an Aβ(X-38 . . . 43) oligomer or derivative of the invention to the subject; the use of an agent capable of binding to an Aβ(X-38 . . . 43) oligomer or derivative of the invention for preparing a composition for diagnosing an amyloidosis and corresponding methods of diagnosing an amyloidosis which comprises providing a sample from the subject suspected of having the amyloidosis, contacting the sample with an agent capable of binding to an Aβ(X-38 . . . 43) oligomer or derivative of the invention for a time and under conditions sufficient for the formation of a complex comprising the agent and an antigen, the presence of the complex indicating the subject has the amyloidosis.

### BRIEF DESCRIPTION OF DRAWINGS

FIG. 1 shows a size exclusion chromatogram (SEC) on Superose 6 of Aβ(20-42) HMW oligomer obtained in A) example 1; B) example 2; C) example 3; D) example 4; E) example 5; F) example 6; G) example 7; H) example 8; I) example 9; J) example 10.

FIG. 2 shows a size exclusion chromatogram (SEC) on Superose 6 of the preparation obtained in in A) example 12a; B) example 12b; C) example 12c; D) example 12d; E) example 12e; F) example 12f.

FIG. 3 shows an SDS-PAGE: lane 1) molecular weight marker; lane 2) 500 ng in Aβ(20-42) HFIP and diluted in SDS sample buffer; lane 3) 100 ng Aβ(20-42) in HFIP and diluted in SDS sample buffer; lane 4) example 12a, # 1; lane 5) example 12a, # 2; lane 6) example 12b, #1; lane 7) example 12b, #2; lane 8) example 12c, #1; lane 9) example 12c, #2; lane 10) example 12d, #1; lane 11) example 12d, #2; lane 12) example 12e, #1; lane 13) example 12e, #2; lane 14) example 12f, #1 lane 15) example 12f, #2.

FIG. 4 shows a size exclusion chromatogram (SEC) on Superose 6 of Aβ(20-42) HMW oligomer preparations obtained with different initial HFIP concentrations: A) 5% HFIP, B) 10% HFIP, C) 25% HFIP, D) 50% HFIP.

FIG. 5 shows dot blot analyses of the reactivity with
1. Aβ(1-40) peptide, dissolved in 0.1% NaOH,
2. Aβ(1-42) peptide, dissolved in 0.1% NaOH,
3. Aβ(1-42) globulomer,
4. Aβ(12-42) truncated globulomer,
5. Aβ(20-42) truncated globulomer,
6. Aβ(20-42) HMW oligomer (from example 1),
7. Aβ(1-42) fibril preparation,
8. sAPPa (Sigma),
9. Aβ(20-42) peptide, dissolved in 50% HFIP
   of antisera from 3 rabbits A), B) and C) before (left panel) and 28 days after (right panel) immunization with Aβ(20-42) HMW oligomer.

FIG. 6 shows dot blot analyses of the reactivity with
1. Aβ(1-40) peptide, dissolved in 0.1% NaOH,
2. Aβ(1-42) peptide, dissolved in 0.1% NaOH,
3. Aβ(1-42) globulomer,
4. Aβ(12-42) truncated globulomer,
5. Aβ(20-42) truncated globulomer,
6. Aβ(20-42) HMW oligomer (from example 1),
7. Aβ(1-42) fibril preparation,
8. sAPPa (Sigma),
9. Aβ(20-42) peptide, dissolved in 50% HFIP of antisera from 2 mice (left and right panel) after immunization with Aβ(20-42) HMW oligomer.

FIG. 7 shows dot blot analyses of the reactivity with
1. Aβ(1-40) peptide, dissolved in 0.1% NaOH,
2. Aβ(1-42) peptide, dissolved in 0.1% NaOH,
3. Aβ(1-42) globulomer,
4. Aβ(12-42) truncated globulomer,
5. Aβ(20-42) truncated globulomer,
6. Aβ(20-42) HMW oligomer (from example 1),
7. Aβ(1-42) fibril preparation,
8. sAPPa (Sigma),
9. Aβ(20-42) peptide, dissolved in 50% HFIP
   of antisera from 3 rabbits A), B) and C) 56 days after repeated immunization with Aβ(20-42) HMW oligomer.

FIG. 8 shows dot blot analyses of the reactivity with
1. Aβ(1-42) peptide, dissolved in 0.1% NaOH,
2. Aβ(1-42) globulomer,
3. Aβ(20-42) truncated globulomer,
4. Aβ(20-42) HMW oligomer of example 2,
5. Aβ(20-42) HMW oligomer of example 3,
6. Aβ(20-42) HMW oligomer of example 4,
7. Aβ(20-42) HMW oligomer of example 5
8. Aβ(22-42) HMW oligomer of example 6
9. Aβ(20-42) peptide, dissolved in 50% HFIP,
10. Aβ(22-42) peptide, dissolved in 50% HFIP, of
A) monoclonal antibodies 7C6, 7E5, 4D10, and 5F7 (raised against the Aβ(20-42) truncated globulomer);
B) polyclonal antisera from rabbits immunized with Aβ(20-42) HMW oligomer of example 1 (rabbit #2687 and #2688);
C) polyclonal antisera from rabbits immunized with Aβ(20-42) truncated globulomer (rabbit #5598 and #5600);
D) polyclonal antisera from mice immunized with Aβ(20-42) HMW oligomer of example 2 (mouse #1 and #2); and
E) polyclonal antisera from APP/Lo mice immunized with Aβ(20-42) truncated globulomer (mouse #TAF.WB 993, #TAF.WB 963).

FIG. 9 is a bar diagram which shows the reactivity of monoclonal antibodies 8F5, 5F7, 4D10, 7C6, and control (IgG2a/AP) with of A) Aβ(20-42) truncated globulomer, and B) Aβ(20-42) HMW oligomer of example 3 in an immunoprecipitation assay with relative SELDI-MS quantification.

FIG. 10 is a bar diagram which shows the reactivity of A) polyclonal rabbit immune sera BA197 (Aβ(1-42) globulomer immunization), 5600 (Aβ(20-42) truncated globulomer immunization) and 2687 (Aβ(20-42) HMW oligomer immunization) and B) CSF of control and Alzheimer Disease patients with in vitro generated Aβ(1-42) monomer (NaOH or NH₄OH) and Aβ(20-42) truncated globulomer.

FIG. 11 shows the results of ELISA assays for binding to A) Aβ(1-42) peptide, B) Aβ(20-42) truncated globulomer, and C) Aβ(20-42) HMW oligomer, using the monoclonal antibody 6E10 (A) or 7C6 (B, C) as detection antibody and the following antisera as capture antibody:
BA 197: from rabbit after immunization with Aβ(20-42) globulomer 5598: from rabbit after immunization with Aβ(20-42) globulomer, 5600: from rabbit after immunization with Aβ(20-42) globulomer,
2687-1: from rabbit after immunization with Aβ(20-42) HMW oligomer of example 1,
2687-2: from rabbit after immunization with Aβ(20-42) HMW oligomer of example 1,
2687-pre: from rabbit before immunization with Aβ(20-42) HMW oligomer.

FIG. 12 is A) a bar diagram which shows the amount of Aβ(20-42) HMW oligomer formed with different ampiphatic agents and B) dot blot analyses of the reactivity of monoclonal antibodies 7C6 and 7E5 with
1. Aβ(20-42) peptide+0.5% SDS (pH 7.4),
2. Aβ(20-42) peptide+0.2% Triton X100 (pH 7.4),
3. Aβ(20-42) peptide+0.2% Tergitol NP-40 (pH 7.4),
4. Aβ(20-42) peptide+0.5% Lauric acid (pH 7.4),
5. Aβ(20-42) peptide+0.5% N-Lauroylsarcosine (pH 7.4),
6. Aβ(20-42) peptide+0.5% CHAPS (pH 7.4),
7. Aβ(20-42) peptide+0.25% DDAP (pH 7.4),
8. Aβ(20-42) peptide+0.5% Dodecylamine (pH 7.4),
9. Aβ(20-42) peptide+0.5% Lauric acid (pH 8.7),
10. Aβ(20-42) peptide+0.5% Dodecylamine (pH 10.0),
11) Aβ20-42 peptide+¼ PBS (pH 7.4).

FIG. 13 shows a size exclusion chromatogram (SEC) on Superose 6 of the preparation obtained in example 15.

FIG. 14 shows the size distribution by volume of Aβ(20-42) HMW oligomer obtained in example 1.

FIG. 15 shows dot blot analyses of the reactivity with
A)
1. Aβ(20-42) HMW oligomer of example 9, and
B)
1. Aβ(20-42) HMW oligomer in buffer at pH 8.5 of example 10, of monoclonal antibody 7C6 (raised against the Aβ(20-42) truncated globulomer).

FIG. 16 shows dot blot analyses (left panels) of the reactivity of either 8F5 or 7C6 monoclonal antibody with (A) Aβ(1-42) globulomer (from reference example 3) and (B) Aβ(20-42) HMW oligomer (from example 9) spotted onto a nitrocellulose membrane (1/10 serial dilution) and UV280 nm chromatograms (right panels) resulting from an affinity purification of 8F5 (dotted line) or 7C6 (solid line) antibody including high salt and low pH gradient elution (grey thin line and arrows underneath) on NHS-sepharose based immobilization of (A) Aβ(1-42) globulomer (from reference example 3) and (B) Aβ(20-42) HMW oligomer (from example 9).

FIG. 17 shows an overlay of the UV280 nm chromatograms within the low pH elution (with peak areas (in mAU×mL) being indicated above the figure) of human plasma affinity purified either by Aβ(1-42) globulomer-Sepharose (dashed line), Aβ(20-42) HMW oligomer-Sepharose (dotted line), or as control, Sepharose only (solid line).

FIG. 18 shows (A) immunoprecipitations of CSF of healthy subjects and PBS extracts of AD subjects with different antibodies (as indicated), analyzed by Western blot against Aβ; and (B) densitometric quantification of the Western blot shown in (A), with white bars representing healthy subjects and black bars AD subjects (note that in order to allow for quantification of faint Aβ peptide (low amount of Aβ peptide on Western blot) a long camera exposure time for chemoluminescence detection was chosen, while other Aβ peptide bands were quantified using a short camera exposure time).

### DETAILED DESCRIPTION OF THE INVENTION

As used herein, the ellipsis A . . . B denotes the set comprising all natural numbers from A to B, including both, e.g. “17 . . . 20” thus denotes the group of the numbers 17, 18, 19 and 20. The hyphen denotes a contiguous sequence of amino acids, i.e., “X-Y” comprises the sequence from amino acid X to amino acid Y, including both. Thus, “A . . . B-C . . . D” comprises all possible combinations between members of these two sets, e.g. “17 . . . 20-40 . . . 42” comprises all of the following: 17-40, 17-41, 17-42, 18-40, 18-41, 18-42, 19-40, 19-41, 19-42, 20-40, 20-41 and 20-42. Unless stated otherwise, all numbers refer to the beginning of the mature peptide, 1 indicating the N-terminal amino acid.

The term “Aβ(X-Y)” here refers to the amino acid sequence from amino acid position X to amino acid position Y of the human amyloid β protein including both X and Y, in particular to the amino acid sequence from amino acid position X to amino acid position Y of the amino acid sequence DAEFRHDSGY EVHHQKLVFF AEDVGSNKGA IIGLMVGGW IAT (corresponding to amino acid positions 1 to 43); or any of its naturally occurring variants, in particular those with at least one mutation selected from the group consisting of A2T, H6R, D7N, A21G (“Flemish”), E22G (“Arctic”), E22Q (“Dutch”), E22K (“Italian”), D23N (“Iowa”), A42T and A42V wherein the numbers are relative to the start of the Aβ peptide, including both position X and position Y; or a sequence with up to three amino acid substitutions none of which may prevent oligomer formation; or a sequence with 1, 2, 3, or more conservative amino acid substitutions, preferably with no amino acid substitutions in the portion from amino acid 12 or X, whichever number is higher, to amino acid 42 or Y, whichever number is lower, more preferably with no amino acid substitutions in the portion from amino acid 20 or X, whichever number is higher, to amino acid 42 or Y, whichever number is lower, and most preferably with no amino acid substitutions in the portion from amino acid 20 or X, whichever number is higher, to amino acid 40 or Y, whichever number is lower, an amino acid substitution herein being any deviation from the canonical sequence that is not found in nature. Moreover, one or more amino acids of the above sequences may be systematically substituted with a D-amino acid of the same type (e.g., D-lysine in place of L-lysine).

For instance, the term “Aβ(20-42)” here refers to the amino acid sequence from amino acid position 20 to amino acid position 42 of the human amyloid β protein including both 20 and 42, in particular to the amino acid sequence F AEDVGSNKGA IIGLMVGGVV IA; or any of its naturally occurring variants, in particular those with at least one mutation selected from the group consisting of A21G (“Flemish”), E22G (“Arctic”), E22Q (“Dutch”), E22K (“Italian”), D23N (“Iowa”), A42T and A42V wherein the numbers are relative to the start of the Aβ peptide, including both 20 and 42; or a sequence with up to three amino acid substitutions none of which may prevent oligomer formation; or a sequence with 1, 2, 3, or more conservative amino acid substitutions, preferably without any additional amino acid substitutions.

For purposes of determining conservative amino acid substitutions, the amino acids can be classified into four main categories—acidic, basic, neutral non-hydrophobic, and hydrophobic—depending primarily on the physical-chemical characteristics of the amino acid side chain.

The term “acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

The term “basic amino acid” refers to a hydrophilic amino acid having a side chain pK value of greater than 7. Basic amino acids typically have positively charged side chains at physiological pH due to association with hydronium ion. Genetically encoded basic amino acids include H is (H), Arg (R) and Lys (K).

The term “neutral non-hydrophobic” refers to a neutral and non-hydrophobic amino acid having a side chain that is uncharged at physiological pH, but which has at least one bond in which the pair of electrons shared in common by two atoms is held more closely by one of the atoms. Genetically encoded neutral non-hydrophobic amino acids include Gly (G), Thr (T), Ser (S), Asn (N), Gln (O) and Cys (C).

The term “hydrophobic amino acid” refers to an amino acid exhibiting a hydrophobicity of greater than zero according to the normalized consensus hydrophobicity scale of Eisenberg, 1984, J. Mol. Biol. 179: 1.25-142. Genetically encoded hydrophobic amino acids include Ala (A), Val (V), Leu (L), Ile (I), Met (M), Phe (F), Tyr (Y), Trp (W), and Pro (P).

It will be recognized that in certain embodiments of the invention, the amino acid substitutions are conservative, i.e., the replacing amino acid residue has physical and chemical properties that are similar to the amino acid residue being replaced, i.e., replacing and replaced amino acid residue belong to the same group as defined herein. Especially preferred conservative amino acids substitution groups are: valine-leucine-isoleucine, phenylalanine-tyrosine, lysine-arginine, alanine-valine, and asparagine-glutamine.

The term “Aβ(X-Y) oligomer” here refers to a non-covalent association of Aβ(X-Y) peptides as defined herein, possessing homogeneity and distinct physical characteristics. According to one aspect, Aβ(X-Y) oligomers are stable, non-fibrillar, oligomeric assemblies of Aβ(X-Y) peptides.

According to a particular embodiment, the invention relates to Aβ(X-38 . . . 43) oligomers and derivatives wherein X is 15, 18, 19, 20, 21 or 22. More specifically, the invention relates to Aβ(X-38 . . . 43) oligomers and derivatives wherein Aβ(X-38 . . . 43) is Aβ(X-42) or Aβ(X-40), with X being as defined herein. Specific examples are Aβ(X-38 . . . 43) oligomers and derivatives wherein Aβ(X-38 . . . 43) is Aβ(20-42).

The oligomers and derivatives of the invention can be characterized as having a high molecular weight in size exclusion chromatography.

Size exclusion chromatography is a method well-known in the art.

A high molecular weight in size exclusion chromatography is an apparent molecular weight of at least 200 kDa, at least 300 kDa, at least 400 kDa, or at least 500 kDa, in particular when the size exclusion chromatography is carried out on a Superose 6 HR 10/300 GL column (e.g. GE Health Care, catalogue no. 17-5172-01) using as mobile phase a buffer composed of 20 mM NaH₂PO₄, 140 mM NaCl, pH 7.4 and 1% SDS with a flow-rate of 0.5 ml/min.

According to a particular embodiment, the oligomers and derivatives of the invention are soluble in aqueous media (e.g. an aqueous solution of 5 mM NaH₂PO₄ and 35 mM NaCl, more specifically an aqueous solution of 5 mM NaH₂PO₄ and 35 mM NaCl comprising an amphipatic agent in a concentration as indicated below or an aqueous solution of 5 mM NaH₂PO₄ and 35 mM NaCl having a pH of 8 to 10, 8.0 to 9.5, or 8.0 to 9.0). Solubilities of at least 0.1, 1, or 5 mg protein per mL solution are expedient. Soluble oligomers of the invention also include solubilized oligomers. Solubility can be checked by centrifugation. An oligomer is soluble if it does not precipitate upon centrifugation at 10,000×g and a temperature in the range of 10 to 40° C., e.g. at 37° C.

According to another aspect, the oligomers and derivatives of the invention can be characterized as having a size in the range of about 5 nm to about 500 nm, when determined by photon correlation spectroscopy at 25° C. using a backscatter angle of 173°. Usually, the oligomers or derivatives in an oligomer or derivative preparation of the invention will have a size distribution, with one or more than one frequence maximum in the indicated range. According to a particular embodiment, oligomers or derivatives having a size in the range of about 5 nm to about 50 nm are more frequent than oligomers or derivatives having a size greater than about 50 nm, when determined by photon correlation spectroscopy at 25° C. using a backscatter angle of 173°.

According to a particular embodiment, the oligomers and derivatives of the invention are characterized by their reactivity with particular antibodies. Such antibodies include in particular antibodies having a binding affinity to an Aβ(20-42) truncated globulomer that is greater than the binding affinity of the antibody to an Aβ(1-42) globulomer.

The term “Aβ(X-Y) globulomer” (Aβ(X-Y) globular oligomer) here refers to a soluble, globular, non-covalent association of Aβ(X-Y) peptides as defined herein, possessing homogeneity and distinct physical characteristics. According to one aspect, Aβ(X-Y) globulomers are stable, non-fibrillar, oligomeric assemblies of Aβ(X-Y) peptides. In contrast to monomer and fibrils, these globulomers are characterized by defined assembly numbers of subunits (e.g. early assembly forms, n=4-6, “oligomers A”, and late assembly forms, n=12-14, “oligomers B”, as described in WO2004/067561). The globulomers have a 3-dimensional globular type structure (“molten globule”, see Barghorn et al., 2005, J Neurochem, 95, 834-847). They may be further characterized by one or more of the following features:
cleavability of N-terminal amino acids X-23 with promiscuous proteases (such as thermolysin or endoproteinase GIuC) yielding truncated forms of globulomers;
non-accessibility of C-terminal amino acids 24-Y with promiscuous proteases and antibodies;

truncated forms of these globulomers maintain the 3-dimensional core structure of said globulomers with a better accessibility of the core epitope Aβ(20-Y) in its globulomer conformation.

The term “Aβ(X-Y) truncated globulomer” here refers to a truncated form of Aβ(X-Y) globulomer which can be obtained by subjecting Aβ(X-Y) globulomer to limited proteolytic digestion. More specifically, Aβ(X-Y) truncated globulomers include N-terminally truncated forms wherein X is selected from the group consisting of the numbers 2 . . . 24, with X preferably being 20 or 12, and Y is as defined herein, which are obtainable by truncating Aβ(1-Y) globulomers by treatment with appropriate proteases. For instance, an Aβ(20-42) globulomer can be obtained by subjecting an Aβ(1-42) globulomer to thermolysin proteolysis, and an Aβ(12-42) globulomer can be obtained by subjecting an Aβ(1-42) globulomer to endoproteinase GIuC proteolysis. When the desired degree of proteolysis is reached, the protease is inactivated in a generally known manner. The resulting globulomers may then be isolated following the procedures already described herein and, if required, processed further by further work-up and purification steps. A detailed description of said processes is disclosed in WO 2004/067561, which is incorporated herein by reference.

For the purposes of the present invention, an Aβ(1-42) globulomer is in particular the Aβ(1-42) globulomer as described in reference example 3 herein; an Aβ(20-42) truncated globulomer is in particular the Aβ(20-42) truncated globulomer as described in reference example 5 herein, and an Aβ(12-42) truncated globulomer is in particular the Aβ(12-42) truncated globulomer as described in reference example 4 herein.

Antibodies having a binding affinity to an Aβ(20-42) truncated globulomer that is greater than the binding affinity of the antibody to an Aβ(1-42) globulomer are described in WO 2007/062852 and include, for instance, a monoclonal antibody selected from the group consisting of 7C6, 7E5, 4D10 and 5F7.

According to a particular embodiment, the said antibody binds to the Aβ(X-38 . . . 43) oligomers and derivatives of the present invention with a K_{D} in the range of at least 1×10⁻⁶ M.

Preferably, the antibodies bind to the Aβ(X-38 . . . 43) oligomers and derivatives of the present invention with high affinity, for instance with a K_{D} of 1×10⁻⁷ M or greater affinity, e.g. with a K_{D} of 3×10⁻⁸ M or greater affinity, with a K_{D} of 1×10⁻⁸ M or greater affinity, e.g. with a K_{D} of 3×10⁻⁹ M or greater affinity, with a K_{D} of 1×10⁻⁹ M or greater affinity, e.g. with a K_{D} of 3×10⁻¹⁰ M or greater affinity, with a K_{D} of 1×10⁻¹⁰ M or greater affinity, e.g. with a K_{D} of 3×10⁻¹¹ M or greater affinity, or with a K_{D} of 1×10⁻¹¹ M or greater affinity.

The term “greater affinity” here refers to a degree of interaction where the equilibrium between unbound antibody and unbound oligomer or derivative on the one hand and antibody-oligomer/derivative complex on the other is further in favour of the antibody-oligomer/derivative complex. Likewise, the term “smaller affinity” here refers to a degree of interaction where the equilibrium between unbound antibody and unbound oligomer or derivative on the one hand and antibody-oligomer/derivative complex on the other is further in favour of the unbound antibody and unbound oligomer or derivative. The term “greater affinity” is synonymous with the term “higher affinity” and term “smaller affinity” is synonymous with the term “lower affinity”.

The binding affinities of antibodies (monoclonal or polyclonal) to a given antigen (such as the Aβ(X-38 . . . 43) oligomers and derivatives of the present invention) may be evaluated by using standardized in-vitro immunoassays such as ELISA, dot blot or BIAcore analyses (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnsson, B., et al. (1991) Anal. Biochem. 198:268-277.

According to a particular embodiment, the affinities defined herein refer to the values obtained by performing a dot blot as described in example 20 and evaluating it by densitometry. According to a particular embodiment of the invention, determining the binding affinity by dot blot comprises the following: a certain amount of the antigen or, expediently, an appropriate dilution thereof, for instance in 20 mM NaH₂PO₄, 140 mM NaCl, pH 7.4, 0.2 mg/ml BSA to an antigen concentration of, for example, 100 μmol/μl, 10 μmol/μl, 1 μmol/μl, 0.1 μmol/μl and 0.01 μmol/μl, is dotted onto a nitrocellulose membrane, the membrane is then blocked with milk to prevent unspecific binding and washed, then contacted with the antibody of interest followed by detection of the latter by means of an enzyme-conjugated secondary antibody and a colorimetric reaction; at defined antibody concentrations, the amount of antibody bound allows affinity determination. Thus the relative affinity of two different antibodies to one target, or of one antibody to two different targets, is here defined as the relation of the respective amounts of target-bound antibody observed with the two antibody-target combinations under otherwise identical dot blot conditions. Unlike a similar approach based on Western blotting, the dot blot approach will determine an antibody's affinity to a given target in the latter's natural conformation; unlike the ELISA approach, the dot blot approach does not suffer from differences in the affinities between different targets and the matrix, thereby allowing for more precise comparisons between different targets.

The term “K_{d}”, as used herein, is intended to refer to the dissociation constant of a particular antibody-antigen interaction as is known in the art.

Aβ(X-38 . . . 43) oligomers and derivatives of the present invention that react with globulomer-specific antibodies are believed to display at least one globulomer epitope. Therefore, the oligomers and derivatives of the present invention are capable of eliciting an immune response having a similar profil as the immune response elicited when Aβ(20-42) truncated globulomers or other truncated globulomers are used as immunogen.

The term “epitope” includes any polypeptide determinant capable of specific binding to an immunoglobulin. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. An epitope may also be recognized by other binding agents than immunoglobulins.

According to a further particular embodiment, the oligomers and derivatives of the invention are characterized by their ability of eliciting such a particular immune response, for instance if a mammal, e.g. a rabbit or a mouse, is immunized with an oligomer or derivative of the invention.

An immune response may be regarded as a mixture of antibodies resulting from challenging (immunizing) a host with an antigen (the immunogen). Said mixture of antibodies can be obtained from the host and is hereinafter referred to as the polyclonal antiserum.

In one aspect, such a particular immune response, i.e., the corresponding polyclonal antiserum, is characterized by comprising an antibody having a binding affinity to an oligo-mer or derivative of the invention or to an Aβ(20-42) truncated globulomer that is greater than the binding affinity of the antibody to at least one Aβ form selected from the group consisting of monomeric Aβ(1-42), monomeric Aβ(1-40), monomeric Aβ(20-42), fibrillomeric Aβ(1-42), fibrillomeric Aβ(1-40) and Aβ(1-42) globulomer.

According to a particular embodiment, the immune response, i.e., the corresponding polyclonal antiserum, is characterized by having an affinity to an oligomer or derivative of the invention or to an Aβ(20-42) truncated globulomer which is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antiserum to at least one Aβ form selected from the group consisting of monomeric Aβ(1-42), monomeric Aβ(1-40), monomeric Aβ(20-42), fibrillomeric Aβ(1-42), fibrillomeric Aβ(1-40) and Aβ(1-42) globulomer.

The term “Aβ(X-Y) monomer” or “monomeric Aβ(X-Y)” here refers to the isolated form of the Aβ(X-Y) peptide, preferably a form of the Aβ(X-Y) peptide which is not engaged in essentially non-covalent interactions with other Aβ peptides. Practically, the Aβ(X-Y) monomer is usually provided in the form of an aqueous solution. In a particularly preferred embodiment of the invention, the aqueous monomer solution contains 0.05% to 0.2%, more preferably about 0.1% NH₄OH. In another particularly preferred embodiment of the invention, the aqueous monomer solution contains 0.05% to 0.2%, more preferably about 0.1% NaOH. When used (for instance for determining the binding affinities of the antibodies of the present invention), it may be expedient to dilute said solution in an appropriate manner. Further, it is usually expedient to use said solution within 2 hours, in particular within 1 hour, and especially within 30 minutes after its preparation.

More specifically, the term “Aβ(1-40) monomer” here refers to an Aβ(1-40) monomer preparation as described in reference example 1 herein, and the term “Aβ(1-42) monomer” here refers to an Aβ(1-42) preparation as described in reference example 2, 10 or 11 herein.

In another aspect, such an immune response is characterized by comprising an antibody having a binding affinity to an oligomer or derivative of the invention or to an Aβ(20-42) truncated globulomer that is greater than the binding affinity of the antibody to an Aβ(1-42) globulomer.

The term “fibril” here refers to a molecular structure that comprises assemblies of non-covalently associated, individual Aβ(X-Y) peptides, which show fibrillary structure in the electron microscope, which bind Congo red and then exhibit birefringence under polarized light and whose X-ray diffraction pattern is a cross-6 structure.

In another aspect of the invention, a fibril is a molecular structure obtainable by a process that comprises the self-induced polymeric aggregation of a suitable Aβ peptide in the absence of detergents, e.g. in 0.1 M HCl, leading to the formation of aggregates of more than 24, preferably more than 100 units. This process is well known in the art. Expediently, Aβ(X-Y) fibrils are used in the form of an aqueous solution. In a particularly preferred embodiment of the invention, the aqueous fibril solution is made by dissolving the Aβ peptide in 0.1% NH₄OH, diluting it 1:4 with 20 mM NaH₂PO₄, 140 mM NaCl, pH 7.4, followed by readjusting the pH to 7.4, incubating the solution at 37° C. for 20 h, followed by centrifugation at 10,000 g for 10 min and resuspension in 20 mM NaH₂PO₄, 140 mM NaCl, pH 7.4.

The term “Aβ(X-Y) fibril” here refers to a fibril consisting essentially of Aβ(X-Y) subunits, where it is preferred if on average at least 90% of the subunits are of the Aβ(X-Y) type, more preferred if at least 98% of the subunits are of the Aβ(X-Y) type, and most preferred if the content of non-Aβ(X-Y) peptides is below the detection threshold.

More specifically, the term “Aβ(1-42) fibril” here refers to a Aβ(1-42) fibril preparation as described in reference example 6 herein.

The present invention also relates to a purified Aβ(X-38 . . . 43) oligomer of the invention or a purified derivative thereof. According to one embodiment of the present invention, a purified Aβ(X-38 . . . 43) oligomer or derivative thereof is one which has a purity of more than 80% by weight of total Aβ peptide, preferably of more than 90% by weight of total Aβ peptide, preferably of more than 95% by weight of total Aβ peptide.

The invention also relates to a composition comprising the Aβ(X-38 . . . 43) oligomer or derivative thereof, preferably a composition comprising Aβ(X-38 . . . 43) oligomer or a derivative thereof, wherein the amount of the oligomer or the derivative is at least 0.3 mg/ml of the composition, preferably at least 0.7 mg/ml of the composition and more preferably at least 1.0 mg/ml of the composition.

It may be expedient to modify the oligomers of the invention. For instance, diagnostic applications may require labelling the oligomers. Also, in active immunization it may be of advantage to stabilize the oliogmers by cross-linking or to attach other moieties which prove expedient in active immunization applications. The invention, therefore, includes derivatives of the Aβ(X-38 . . . 43) oligomer of the invention.

Such derivatives in particular include cross-linked, preferably chemically cross-linked, Aβ(X-38 . . . 43) oligomers. Suitable crosslinkers are known to the skilled worker and are usually bifunctional reagents such as formaldehyde, glutardialdehyde, disuccinimidyl suberate, dithiobis(succinimidyl propionate), disuccinimidyl tartrate, disulfosuccinimidyl tartrate, dimethyl adipimidate, dimethyl pimelidate, dimethyl suberimidate, dimethyl 3,3′-dithiobispropionimidate, N-γ-maleinimidobutyloxysuccinimide ester, succinimidyl 4(N-maleinimidomethyl)cyclohexane-1-carboxylate, N-succinimidyl (4-iodacetyl)aminobenzoate and N-succinimidyl 3-(2-pyridyldithio)propionate.

Further derivatives of the Aβ(X-38 . . . 43) oligomer include Aβ(X-38 . . . 43) oligomers which are labelled by being covalently linked to a group that facilitates detection, preferably a fluorophore, e.g. fluorescein isothiocyanate, phycoerythrin, Aequorea victoria fluorescent protein, Dictyosoma fluorescent protein or any combination or fluorescence-active derivative thereof; a chromophore; a chemoluminophore, e.g. luciferase, preferably Photinus pyralis luciferase, Vibrio fischeri luciferase, or any combination or chemoluminescence-active derivative thereof; an enzymatically active group, e.g. peroxidase, e.g. horseradish peroxidase, or any enzymatically active derivative thereof; an electron-dense group, e.g. a heavy metal containing group, e.g. a gold containing group; a hapten, e.g. a phenol derived hapten; a strongly antigenic structure, e.g. peptide sequence predicted to be antigenic, e.g. predicted to be antigenic by the algorithm of Kolaskar and Tongaonkar; a molecule which helps elicit an immune response to the oligomer, e.g., serum albumin, ovalbumin, keyhole limpet hemocyanin, thyroglobulin, a toxoid from bacteria such as tetanus toxoid and diphtheria toxoid, a naturally occurring T cell epitope, a naturally occurring T helper cell epitope; an artificial T-cell epitope such as the pan DR epitope (“PADRE”; WO 95/07707), or another immunostimulatory agent, e.g., mannan, tripalmitoyl-S-glycerine cysteine, and the like; an aptamer for another molecule; a chelating group, e.g. hexahistidinyl; a natural or nature-derived protein structure mediating further specific protein-protein interactions, e.g. a member of the fos/jun pair; a magnetic group, e.g. a ferromagnetic group; or a radioactive group, e.g. a group comprising 1H, 14C, 32P, 35S or 1251 or any combination thereof. Such labelling groups and methods for attaching them to proteins are known in the art. With a view to avoiding the unfavored pro-inflammatory immune response Th1-pathway, oligomer derivatives comprising a molecule which is capable of directing the immune response to the anti-inflammatory pathway (Th2-pathway), e.g. molecules comprising a B cell epitope such as PADRE are expected to provide particular advantages in active immunization (see also, Petrushina I., et al., The Journal of Neuroscience 2007, 27(46): 12721-12731; Woodhouse A., et al., Drugs Aging 2007; 24(2): 107-119).

In another particular embodiment of the invention, the derivative of the Aβ(X-38 . . . 43) oligomer is a Aβ(X-38 . . . 43) oligomer which comprises at least one monomeric subunit that is flagged by being covalently or by non-covalent high-affinity interaction, preferably covalently linked to a group that facilitates inactivation, sequestration, degradation and/or precipitation, preferably flagged with a group that promotes in vivo degradation, more preferably with ubiquitin.

Further derivatives of the Aβ(X-38 . . . 43) oligomer include Aβ(X-38 . . . 43) oligomers which are cross-linked and labelled or cross-linked and flagged.

Unless stated otherwise, the description of the oligomers of the invention also applies to their derivatives.

The oligomers of the invention and derivatives are obtainable by oligomerization of Aβ(X-38 . . . 43) peptide or a derivative thereof. The oligomerization comprises a noncovalent aggregation of monomeric Aβ(X-38 . . . 43) peptide or a derivative thereof so that the oligomers of the invention can be assumed to be composed of a plurality of Aβ(X-38 . . . 43) peptides or derivatives thereof.

The starting material, i.e. Aβ(X-38 . . . 43) peptide or a derivative thereof, may be prepared by known peptide-synthetic methods or recombinantly. In addition, a number of these proteins are commercially available. The same applies also to muteins and allelic variants of Aβ(X-38 . . . 43) peptide. In a particular embodiment, Aβ(X-38 . . . 43) peptide is synthetic Aβ(X-38 . . . 43) peptide or a derivative thereof.

In a first step, monomeric Aβ(X-38 . . . 43) peptide or a derivative thereof is dissolved in a solvent. Preferably, the solvent is a hydrogen bond-breaking agent. The purpose of this treatment is to provide a solution of the unfolded peptide.

Suitable hydrogen bond-breaking agents are known in the art. These include organic compounds such as 1,1,1,3,3,3-hexafluoro-2-propanol (HFIP) and aqueous solutions of bases such as sodium hydroxide, potassium hydroxide, formic acid, 2,2,2-trifluoroethanol (TFE), urea and guanidinium chloride.

It is of advantage if the organic hydrogen bond-breaking agent is volatile so that it can be removed from the oligomer preparation by evaporation.

It is of further advantage if the bond-breaking agent is an acid or base that can easily be neutralized.

According to a particular embodiment, the hydrogen bond-breaking agent is HFIP.

According to a further particular embodiment, the hydrogen bond-breaking agent is an aqueous solution of sodium hydroxide. A concentration of sodium hydroxide in the range from 0.025% by weight to 0.25% by weight, from 0.05% by weight to 0.15% by weight, or from 0.075% by weight to 0.125% by weight proves expedient. For instance, an aqueous solution comprising about 0.1% by weight of sodium hydroxide can be expediently used.

In order to help the Aβ(X-38 . . . 43) peptide dissolving in the hydrogen bond-breaking agent the mixture may be subjected to agitation, e.g. shaking. Times of dissolution of a few minutes to a few hours, for example 15 minutes to 2 hours, are sufficient when the temperature is from 22 to 37° C. For instance, the peptide can be expediently dissolved by shaking it in HFIP for about 1 hour or 1.5 hours at about 37° C., or in 0.1% aqueous NaOH for about 1 minute at about 37° C.

The amount of Aβ(X-38 . . . 43) peptide may be such that 2 mg/mL to 50 mg/mL, 5 mg/mL to 40 mg/mL, or 10 mg/mL to 30 mg/mL of peptide are dissolved in the hydrogen bond-breaking agent. For instance, the concentration of Aβ(X-38 . . . 43) peptide dissolved in the hydrogen bond-breaking agent can be expediently adjusted to about 20 mg/mL in HFIP, or mg/mL or 12.5 mg/mL in 0.1% aqueous NaOH.

It is expedient if a clear solution is obtained by dissolving the Aβ(X-38 . . . 43) peptide in the hydrogen bond-breaking agent.

In a further step, an amphipatic agent is added to the solution of Aβ(X-38 . . . 43) peptide in the hydrogen bond-breaking agent. The addition of the amphipatic agent induces the oligomerization of the peptide to give the oligomers. Alternatively, an amphipatic agent is not added to the solution of Aβ(X-38 . . . 43) peptide in the hydrogen bond-breaking agent; in that case, the oligomerization of the peptide to give the oligomers of the invention will take place if the pH is sufficiently high to maintain the peptide in solution.

Amphipathic agents include fatty acids or detergents, some of which are listed in WO2007064917.

For instance, sulfates, in particular alkyl sulfates and alkyl ether sulfates; sulfonates, e.g. sodium dodecyl sulphate (SDS), carboxylic acids, such as fatty acids, e.g. lauric acid, sarcosines, e.g. N-lauroylsarcosin (also known as sarkosyl NL-30 or Gardol®), alkylaryl alcohol polyoxyethylene ethers, such as octylphenol polyoxyethylene ethers, e.g. tert-octylphenol x 9-10E0 (also known as Triton® X100), or alkylaryl nonylphenol polyoxyethylene ethers, e.g. nonylphenol x 20E0 (also known as Tergitol® NP-40), 3-(3-cholamidopropyldimethylammonio-1-propane sulfonate (CHAPS), dodecyl-N,N-dimethyl-3-amino-1-propane sulfonate (DDAP), and amines, in particular alkyl amines, e.g. dodecylamine, can be expediently used as amphipatic agent in the process of the invention. Also, sugar surfactants, in particular polyethoxylated sorbitol esters, such as, for example, polyethoxylated sorbitol fatty acid esters, e.g. polyoxyethylene sorbitan monooleate (also kown as Polysorbat 80 or Tween® 80), can be expediently used as amphipatic agent in the process of the invention.

According to a particular embodiment, the amphipatic agent is added in the form of an aqueous solution comprising the amphipatic agent. Said solution may be buffered. A pH value in the range from 6.0 to 10.0, 6.5 to 9.5, or 7.0 to 9.0 proves expedient. For instance, a buffered aqueous solution having a pH value of about 7.4 or 8.7 can expediently be used. Suitable buffered aqueous solutions are known in the art. For instance, an aqueous solution comprising 5 mM NaH₂PO₄ and 35 mM NaCl can expediently be used.

By adding the aqueous solution to the Aβ(X-38 . . . 43) peptide dissolved in the hydrogen bond-breaking agent the solution is diluted. An amount of aqueous solution added in the range of 5 to 50, 7 to 30, or 8 to 25 times the volume of the solution of the Aβ(X-38 . . . 43) peptide in the hydrogen bond-breaking agent proves expedient. For instance, the amount of aqueous solution to be added can expediently be about 9 or 20 times the volume of the solution of the Aβ(X-38 . . . 43) peptide in the hydrogen bond-breaking agent. However, the amount of aqueous solution added may also be lower, e.g. in the range of 1 to 4 times the volume of the solution of the Aβ(X-38 . . . 43) peptide in the hydrogen bond-breaking agent.

The concentration of amphipatic agent to be chosen depends on the agent used. If SDS is used, a concentration in the range from 0.05 to 0.7% by weight, from 0.075 to 0.4% by weight, or from 0.1 to 0.3% by weight in the incubation mixture proves expedient. For instance, a buffered aqueous solution comprising about 0.2% or 0.25% by weight of SDS can expediently be used. If lauric acid or N-lauroylsarcosin is used, somewhat higher concentrations are expedient, for example in a range from 0.1 to 1.0% by weight, from 0.25 to 0.75% by weight, or from 0.4 to 0.6% by weight. For instance, a buffered aqueous solution comprising about 0.5% by weight of lauric acid or N-lauroylsarcosin can expediently be used. If polyoxyethylene sorbitan monooleate (e.g. Tween® 80) is used, a concentration in the range from 0.05 to 1% by weight, from 0.075 to 0.5% by weight, or from 0.1 to 0.3% by weight in the incubation mixture proves expedient.

Usually, the solution containing the Aβ(X-38 . . . 43) peptide and the buffered aqueous solution are mixed, expediently under agitation, e.g. vortexing.

If the hydrogen bond-breaking agent used is an aqueous solution of a base such as sodium hydroxide, it is expedient that the pH of the mixture is adjusted to a pH in the range from 6.0 to 10.0, 6.5 to 9.5, or 7.0 to 9.0 after the addition of the amphipatic agent. For instance, a pH of about 7.4 proves expedient. If an amphipatic agent is not added, it is expedient that the pH of the mixture is adjusted to a pH in the range from 8.0 to 10.0, 8.0 to 9.5, or 8.0 to 9.0. For instance, a pH of about 8.5 proves expedient.

The adjustment of pH can be effected in a manner known per se. According to a particular embodiment, a suitable amount of an aqueous solution of HCl can be added. For instance, an aqueous solution comprising 2% or 1% by weight of HCl proves expedient.

Before the mixture is incubated to complete oligomer formation, it may be expedient to remove solids from the mixture.

The removal of solids can be effected in manner known per se. According to a particular embodiment, the mixture is centrifuged and the supernatant of the centrifuged mixture is obtained. For instance, centrifugation for about 10 minutes at about 10,000×g proves expedient.

Before solids are removed, the mixture may be pre-incubated for a relatively short period. A few minutes such as 2 minutes to 30 minutes at a temperature in the range from 20 to 37° C. are suitable. For instance, the mixture can be expediently pre-incubated for about 5, 10 or 20 minutes at about room temperature (usually about 22° C.) or 37° C. During this pre-incubation the mixture may be subjected to agitation, e.g. shaking.

Times of incubation for completing oligomer formation may range from a few minutes to a few hours. 5 minutes to 30 hours, 30 minutes to 24 hours, or 1 hour to 3 hours are sufficient when the temperature of incubation is from 15 to 50° C., from 18 to 45° C. or from 20 to 40° C. For instance, oligomer formation is complete if the mixture is uncubated for about 20 hours or 22 hours at about room temperature (usually about 22° C.) or 37° C.

The incubation step may comprise a change in the incubation temperature. A first period of incubation at a first temperature followed by a second period of incubation at a second temperature, the second temperature being inferior to the first temperature, proves expedient. Said first temperature may be in the range from 15 to 50° C., from 18 to 45° C. or from 20 to 40° C. Said second temperature may be in the range from 15 to 50° C., from 18 to 45° C. or from 20 to 40° C. For instance, incubation can expediently comprise incubating at 37° C. followed by incubation at about room temperature (usually about 22° C.).

Usually, it is expedient if the time of incubation at the higher temperature is shorter than the time of incubation at the lower temperature. Said time of incubation at the higher temperature may be in the range from 10 minutes to 10 hours, from 30 minutes to 6 hours or from 1 hour to 5 hours. Said time of incubation at the lower temperature may be in the range from 20 minutes to 30 hours, from 30 min to 24 hours or from 1 hour to 3 hours. For instance, the time of incubation at the first temperature (e.g. at about 37° C.) can be about 4 or 2 hours and the time of incubation at the second temperature (e.g. at about room temperature) can be about 16, 18 or 20 hours, respectively.

According to a particular embodiment, at least a part of the hydrogen bond-breaking agent is allowed to evaporate during incubation if an organic hydrogen bond-breaking agent is used. To this end, the hydrogen bond-breaking agent is expediently a volatile organic compound such as HFIP.

Evaporation can be effected in manner known per se. According to a particular embodiment, the mixture comprising the hydrogen bond-breaking agent is incubated in an open container (e.g. a tube with open lid).

The efficiency of evaporation may be increased by agitation. During the incubation it may therefore be expedient if the mixture is subjected to agitation, e.g. shaking. For instance, shaking may expediently be performed in an orbital shaker, e.g. at about 850 rpm.

The efficiency of evaporation may be also increased by increasing the surface-to-volume ratio of the incubation mixture. It may therefore be expedient if the total volume of the incubation mixture is distributed to a plurality of incubation containers, e.g. tubes. For instance, a total volume of 30 mL can expediently be aliquoted into 60 containers each containing 500 μL.

Evaporation can expediently effected during the period of incubation at the first temperature, as described herein. For instance, the hydrogen bond-breaking agent can expediently be allowed to evaporate during a period of incubation wherein the temperature of incubation is in the range from 15 to 50° C., from 18 to 45° C. or from 20 to 40° and the time of incubation is in the range from 10 minutes to 10 hours, from 30 min to 5 hours or from 1 hour to 3 hours. For instance, the hydrogen bond-breaking agent can expediently be allowed to evaporate during a period of incubation of about 2 hours at about 37° C.

Alternatively, the incubation does not involve a temperature change. This is in particular expedient if hydrogen bond-breaking agent is an aqueous solution of a base such as sodium hydroxide. In this instance, times of incubation for completing oligomer formation may range from 5 min to 30 hours, 30 minutes to 22 hours, or 1 hour to 4 hours when the temperature of incubation is from 15 to 50° C., from 18 to 45° C. or from 20 to 40° C. For instance, the time of incubation can expediently be about 20 hours (2 hours) hours at about 22° C. (37° C.), or, if no amphipatic agent is added, about 20 hours at about 37° C.

Once oligomer formation is complete, it may be expedient to centrifuge the incubation mixture and obtain the supernatant of centrifuged incubation mixture. For instance, centrifugation for about 5 or 10 minutes at about 10,000×g proves expedient.

According to a particular embodiment, the supernatant of centrifuged incubation mixture can then be frozen. For instance, the supernatant of centrifuged incubation mixture can expediently be frozen at −30° C. for 30 minutes. The frozen supernatant is then thawed and optionally the thawed supernatant is again centrifuged (e.g. for 10 minutes at 10,000×g) and the supernatant of centrifuged mixture is obtained.

The oligomer preparation obtainable by this process may be used as such or subjected to further work-up, e.g. in order to concentrate and/or purify the oligomers or derivatives thereof.

According to a particular embodiment, the process of the invention comprises a step of concentrating the incubation mixture.

Concentrating the incubation mixture can be effected in manner known per se. According to a particular embodiment, concentrating is done by ultracentifugation. Ultracentifugation is a method well-known in the art. Ultracentifugation comprising a 10 to 100, a 20 to 80, or a 25 to 50 kDa cut-off proves expedient. For instance, the oligomers or derivatives of the invention can expediently be concentrated by ultracentifugation comprising an about 30 kDa cut-off.

Ultracentrifugation reduces the volume of the incubation mixture while maintaining the amount of oligomer or derivative that is present in the incubation mixture. Thus, it is expedient to reduce the volume to 1 to 40%, 2 to 35%, or 4 to 33%. For instance, the volume of the incubation mixture can expediently be reduced by ultracentrifugation to about 32%, 10% or 5%.

According to a further particular embodiment, the incubation mixture is diluted before being concentrated. This dilution may be expedient to allow for a reduction of the amount of amphipatic agent in the oligomer preparation.

Dilution of the incubation mixture can be effected in manner known per se. According to a particular embodiment, dilution comprises adding a solution. Said solution may be buffered. A pH value in the range from 6.0 to 10.0, 6.5 to 9.5, or 7.0 to 9.0 proves expedient. For instance, a buffered aqueous solution having a pH value of about 7.4 can expediently be used. Suitable buffered aqueous solutions are known in the art. For instance, an aqueous solution comprising 5 mM NaH₂PO₄ and 35 mM NaCl can expediently be used.

In order to allow for a significant reduction of the amount of amphipatic agent in the oligomer preparation it is expedient if dilution comprises reducing the concentration of the amphipatic agent below its critical micellar concentration (CMC). For instance, if the amphipatic agent is SDS and the initial concentration of SDS in the incubation mixture is about 0, 2 or 0,25%, the mixture can expediently be diluted about 2-fold to 20-fold, 3-fold to 15-fold, or 4-fold to 10-fold, e.g. 4-fold (1:3).

It may be expedient to centrifuge the concentrate and obtain the supernatant of centrifuged concentrate. For instance, centrifugation for about 10 minutes at about 10,000×g proves expedient.

According to a particular embodiment, the process of the invention comprises the step of reducing the salt concentration of the incubation mixture or the concentrated incubation mixture.

A reduction of the salt concentration (and of the amphipathic agent, which is particularly important for a use in active immunization) can be effected in manner known per se. According to a particular embodiment, the salt concentration is reduced by subjecting the incubation mixture or the concentrated incubation mixture to dialysis. Dialysis is a method well-known in the art. For instance, dialysis of the incubation mixture or the concentrated incubation mixture can expediently be performed against a solution comprising 5 mM NaH₂PO₄ and 35 mM NaCl. The solution may also comprise amphipatic agent in a suitable amount. During dialysis it may be expedient to replace the solution by a fresh one.

Dialysis is performed until salt reduction is complete. For instance, about 20 hours at about 22° C. prove expedient.

It may further be expedient to centrifuge the dialysate and obtain the supernatant of centrifuged dialysate. For instance, centrifugation for about 10 minutes at about 10,000×g proves expedient.

According to a further particular embodiment, the salt concentration (and/or amphipathic agent) is reduced by subjecting the incubation mixture or the concentrated incubation mixture to HPLC using a desalting column and an aqueous buffered solution as eluent. This desalting method is also a method well-known in the art. For instance, a buffered solution comprising 5 mM NaH₂PO₄, 35 mM NaCl can expediently be used as eluent. The solution may also comprise amphipatic agent in a suitable amount.

Thus, according to a particular embodiment, the invention relates to a process for preparing an Aβ(X-38 . . . 43) oligomer or a derivative thereof, wherein X is selected from the group consisting of the numbers 12 . . . 24, which process comprises
(i) dissolving monomeric Aβ(X-38 . . . 43) peptide or a derivative thereof in a hydrogen bond-breaking agent;
(ii) adding an amphipatic agent, mixing, optionally adjusting pH, pre-incubating, centrifuging, obtaining the supernatant, incubating, centrifuging and obtaining the supernatant;
(iii) diluting, concentrating, centrifuging, and obtaining the supernatant; and
(iv) reducing salt and/or amphipathic agent concentration, centrifuging and obtaining the supernatant.

Derivatization of the oligomers can be performed before or after oligomerization. Accordingly, Aβ peptide or a derivative thereof suitable for being used in said method includes optionally derivatized Aβ(X-38 . . . 43) monomer, X preferably being selected from the group consisting of the numbers 12 . . . 24.

Aβ(X-38 . . . 43) oligomers or derivatives thereof which are obtainable by a process described herein are also an aspect of the present invention.

The Aβ(X-38 . . . 43) oligomers or derivatives of the invention have many utilities. For instance, they can be used in: 1) immunization-based interventional therapies (e.g., oligo-mers or derivatives thereof may be used in active immunization to treat or prevent an amyloidosis); 2) diagnostic testing (e.g., the oligomers or derivatives may be used to diagnose an amyloidosis; 3) providing agents such as antibodies and aptamers that bind to the oligomers or derivatives; and 4) crystallographic or NMR-based structure-based design research for developing agents such as antibodies and aptamers that bind to the oligomers or derivatives.

The term “amyloidosis” here denotes a number of disorders characterized by abnormal folding, clumping, aggregation and/or accumulation of particular proteins (amyloids, fibrous proteins and their precursors) in various tissues of the body. In Alzheimer's disease and Down's syndrome, nerve tissue is affected, and in cerebral amyloid angiopathy (CAA) blood vessels are affected. According to a particular embodiment of the present invention, an amyloidosis is selected from the group consisting of Alzheimer's disease (AD) and the amyloidosis of Down's syndrome.

In active immunization, Aβ(20-42) truncated globulomer was shown to be effective in reversing cognitive defects in Alzheimer Disease transgenic mice. The Aβ(X-38 . . . 43) oligomers or derivatives of the present invention are able to elicit an immune response whose profil is similar to the profil of the immune response elicited by Aβ(20-42) truncated globulomer.

Thus, the invention also relates to the Aβ(X-38 . . . 43) oligomer or derivative as defined herein for therapeutic uses.

In one aspect, the invention relates to therapeutic compositions comprising an Aβ(X-38 . . . 43) oligomer or derivative as defined herein. According to a particular embodiment, said compositions are pharmaceutical compositions which further comprise a pharmaceutical acceptable carrier.

In a further aspect, the invention relates to the use of the Aβ(X-38 . . . 43) oligomer or derivative as defined herein for preparing a pharmaceutical composition for treating or preventing an amyloidosis.

In a preferred embodiment of the invention, the pharmaceutical composition is a vaccine for active immunization.

Accordingly, the invention also relates to a method of treating or preventing an amyloidosis in a subject in need thereof, which comprises administering the Aβ(X-38 . . . 43) oligomer or derivative as defined herein to the subject.

In a preferred embodiment of the invention, administering the Aβ(X-38 . . . 43) oligomer or derivative is for actively immunizing the subject against an amyloidosis.

In the context of active immunization, it is particularly preferred if the Aβ(X-38 . . . 43) oligo-mer or derivative is not able to enter the patient's CNS in significant amounts.

It is also particularly preferred if the pharmaceutical composition comprising the Aβ(X-38 . . . 43) oligomer or derivative is capable of inducing a strong immune response against Aβ oligomers, preferably a strong immune response directed against Aβ oligomers only, more preferably a strong non-inflammatory antibody-based immune response against Aβ oligomers only. Thus, in one embodiment of the invention the pharmaceutical composition comprises an immunological adjuvant, preferably an adjuvant and a signalling molecule, e.g. a cytokine, that directs the immune response towards the non-inflammatory, antibody-based type. Such adjuvants and signalling molecules are well known to those skilled in the art.

It is particularly preferred if the pharmaceutical composition for active immunization is administered via a route selected from the group consisting of the intravenous route, the intramuscular route, the subcutaneous route, the intranasal route, and by inhalation. It is also particularly preferred if the composition is administered by a method selected from injection, bolus infusion and continuous infusion, each of which may be performed once, repeatedly or in regular intervals.

In a particular embodiment of the invention, long-term continuous infusion is achieved by employing an implantable device. In a further particular embodiment of the invention, the composition is applied as an implantable sustained release or controlled release depot formulation. Suitable formulations and devices are known to those skilled in the art. The details of the method to be used for any given route will depend on the stage and severity of the disease and the overall medical parameters of the subject and are preferably decided upon individually at the treating physician's or veterinary's discretion.

In an especially preferred embodiment of the invention, the pharmaceutical composition for active immunization comprises one or more substances selected from the group consisting of pharmaceutically acceptable preservatives, pharmaceutically acceptable colorants, pharmaceutically acceptable protective colloids, pharmaceutically acceptable pH regulators and pharmaceutically acceptable osmotic pressure regulators. Such substances are described in the art.

In line with globulomer hypothesis, it is believed that subjects suffering from an amyloidosis develop an immune response against endogenous globulomer epitopes. As the Aβ(X-38 . . . 43) oligomers of the present invention react with antibodies that are specifically reactive with said epitopes the oligomers are believed to display the same or a very similar epitope.

The invention thus also relates to the Aβ(X-38 . . . 43) oligomer or derivative as defined herein for diagnostic uses.

In one aspect, the invention relates to diagnostic compositions comprising an Aβ(X-38 . . . 43) oligomer or derivative as defined herein. According to a particular embodiment, said compositions are pharmaceutical compositions which further comprise a pharmaceutical acceptable carrier.

In a further aspect, the invention relates to the use of an Aβ(X-38 . . . 43) oligomer or derivative as defined herein for preparing a composition for diagnosing an amyloidosis.

Accordingly, the invention also relates to a method of diagnosing an amyloidosis which comprises providing a sample from the subject suspected of having the amyloidosis, contacting the sample with an Aβ(X-38 . . . 43) oligomer or derivative as defined herein for a time and under conditions sufficient for the formation of a complex comprising the Aβ(X-38 . . . 43) oligomer or derivative and an antibody, the presence of the complex indicating the subject has the amyloidosis. According to a particular embodiment, at least the step of contacting the sample is carried out ex vivo and in particular in vitro.

Thus, the oligomers and derivatives of the present invention may be used in a variety of diagnostic methods and assays.

According to one embodiment, the method of diagnosing an amyloidosis in a patient suspected of having this disease comprises the steps of: a) isolating a biological sample from the patient; b) contacting the biological sample with an oligomer or derivative for a time and under conditions sufficient for the formation of antibody/oligomer/derivative complexes; c) adding a conjugate to the resulting antibody/oligomer/derivative complexes for a time and under conditions sufficient to allow the conjugate to bind to the bound antibody, wherein the conjugate comprises an antibody attached to a signal generating compound capable of generating a detectable signal; and d) detecting the presence of antibodies which may be present in the biological sample by detecting a signal generated by the signal generating compound, the signal indicating a diagnosis of an amyloidosis in the patient. According to a particular embodiment, at least one of steps b), c) and d) is carried out ex vivo and in particular in vitro. According to a further particular embodiment, the method does not comprise step a).

According to a further embodiment, the method of diagnosing an amyloidosis in a patient suspected of having this disease comprises the steps of: a) isolating a biological sample from the patient; b) contacting the biological sample with anti-antibody specific for antibodies in the sample for a time and under conditions sufficient to allow for formation of anti-antibody/antibody complexes; b) adding a conjugate to resulting anti-antibody/antibody complexes for a time and under conditions sufficient to allow the conjugate to bind to bound antibody, wherein the conjugate comprises an oligomer or derivative of the present invention attached to a signal generating compound capable of generating a detectable signal; and c) detecting a signal generated by the signal generating compound, the signal indicating a diagnosis of an amyloidosis in the patient. According to a particular embodiment, at least one of said steps b) and c) is carried out ex vivo and in particular in vitro. According to a further particular embodiment, the method does not comprise step a).

More specifically, as the oligomers and derivatives of the present invention display the globulomer epitope and the globulomer epitope is believed to be an endogenous antigen which gives rise to an endogenous immune response, diagnosis of amyloidoses can be related to the determination of the presence of auto-antibodies which specifically bind to the Aβ(X-38 . . . 43) oligomers and derivatives of the invention.

The invention thus also relates to the use of the Aβ(X-38 . . . 43) oligomer or derivative as defined herein for preparing a composition for detecting in a subject auto-antibodies that bind to the oligomer or derivative. Accordingly, the invention also relates to a method of detecting auto-antibodies that bind to the Aβ(X-38 . . . 43) oligomer or derivative in a subject, which method comprises administering to the subject Aβ(X-38 . . . 43) oligomer or derivative as defined herein and detecting a complex formed by the antibody and the oligomer or derivative, the presence of the complex indicating the presence of the auto-antibodies. In a particular embodiment of the invention, the subject is suspected of having any form of amyloidosis, e.g. Alzheimer's disease, and detecting auto-antibodies is for diagnosing the presence or absence of any form of amyloidosis, e.g. Alzheimer's disease, in the subject.

The invention also relates to the use of the Aβ(X-38 . . . 43) oligomer or derivative as defined herein for detecting auto-antibodies that bind to the oligomer or derivative in a sample. Accordingly, the invention also refers to a method of detecting auto-antibodies that bind to the Aβ(X-38 . . . 43) oligomer or derivative in a sample, which method comprises contacting the sample with the Aβ(X-38 . . . 43) oligomer or derivative as defined herein and detecting a complex formed by the antibody and the oligomer or derivative, the presence of the complex indicating the presence of the auto-antibodies. According to a particular embodiment, at least the step of contacting the sample is carried out ex vivo and in particular in vitro. In a preferred embodiment of the invention, the sample is derived from a subject suspected of having an amyloidosis, e.g. Alzheimer's disease, and detecting the auto-antibodies is for diagnosing the presence or absence of the amyloidosis, e.g. Alzheimer's disease in the subject. Suitable samples include biological fluids which may be tested by the aforesaid method. These include plasma, whole blood, dried whole blood, serum, cerebrospinal fluid or aqueous or organo-aqueous extracts of tissues and cells.

It is particularly preferred if the subject suspected of having an amyloidosis is a subject having the amyloidosis or having an increased risk of getting the amyloidosis.

According to a particular embodiment of the invention, detecting auto-antibodies as described herein further comprises a pre-treatment of the preparation (sample) which causes dissociation of auto-antibody/antigen complexes. A method comprising such a pre-treatment may therefore be used in order to determine the total amount of auto-antibodies present in the preparation (sample) while a method not comprising said pre-treatment may be used in order to determine the amount of auto-antibodies which can still bind to the antigen. Further, both methods will allow to indirectly determine the amount of complexed auto-antibodies.

Conditions suitable for inducing dissociation of auto-antibody/antigen complexes are known to the skilled person. For instance, treating the preparation (sample) with acid, e.g., using a buffer such that the pH of the resulting preparation (sample) is in the range of 1 to 5, preferably in the range of 2 to 4 and in particular in the range of 2 to 3, may be expedient. Suitable buffers include salts in a physiological concentration, e.g. NaCl and acetic acid. A method for separation of antibody/antigen complexes has been described in WO2005/037209, which is incorporated herein in its entirety.

Briefly, dissociating the antibody from the antigen in the antibody/antigen complex comprises the steps of: contacting the sample containing an antibody/antigen complex with a dissociation buffer; incubating the sample; and optionally concentrating the sample.

The dissociation buffer may be a PBS buffer which has a pH in the range as indicated herein. For instance a PBS buffer containing about 1.5% BSA and 0.2 M glycine-acetate pH 2.5, or 140 mM NaCl and 0.58% acetic acid is suitable.

Incubation for several minutes, for instance such as 10 to 30, e.g., 20 minutes at a temperature in the range of 20 to 40° C. has proven sufficient.

Concentration can be achieved in a manner known per se, for instance by passing the sample over a Centriprep YM30 (Amincon Inc.).

In one embodiment of the present invention, the Aβ(X-38 . . . 43) oligomer or derivative, or a portion thereof is coated on a solid phase. The sample (e.g., whole blood, cerebrospinal fluid, serum, etc.) is then contacted with the solid phase. If the antibodies, e.g. the auto-antibodies, are present in the sample, such antibodies bind to the Aβ(X-38 . . . 43) oligomer or derivative on the solid phase and are then detected by either a direct or indirect method. The direct method comprises simply detecting presence of the complex itself and thus presence of the antibodies. In the indirect method, a conjugate is added to the bound antibody. The conjugate comprises a second antibody, which binds to the first bound antibodies, attached to a signal-generating compound or label. Should the second antibody bind to a bound first antibody, the signal-generating compound generates a measurable signal. Such a signal then indicates presence of the first antibodies in the sample.

Examples of solid phases used in diagnostic immunoassays are porous and non-porous materials, latex particles, magnetic particles, microparticles (see U.S. Pat. No. 5,705,330), beads, membranes, microtiter wells and plastic tubes. The choice of the solid phase material and the method of labeling the antigen or antibodies present in the conjugate, if desired, are determined based upon desired assay format performance characteristics.

As noted herein, the conjugate (or indicator reagent) will comprise an antibody (or perhaps anti-antibodies, depending upon the assay), attached to a signal-generating compound or “label”. This signal-generating compound or label is itself detectable or may be reacted with one or more additional compounds to generate a detectable product. Examples of signal-generating compounds are described herein and in particular include chromophores, radioisotopes (e.g., 125I, 131I, 32P, 3H, 35S and 14C), chemiluminescent compounds (e.g., acridinium), particles (visible or fluorescent), nucleic acids, complexing agents, or catalysts such as enzymes (e.g., alkaline phosphatase, acid phosphatase, horseradish peroxidase, beta-galactosidase and ribonuclease). In the case of enzyme use (e.g., alkaline phosphatase or horseradish peroxidase), addition of a chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

Kits are also included within the scope of the present invention. More specifically, the present invention includes kits for determining the presence of antibodies such as auto-antibodies in a subject. In particular, a kit for determining the presence of said antibodies in a sample comprises a) a Aβ(X-38 . . . 43) oligomer or derivative as defined herein; and optionally b) a conjugate comprising an antibody attached to a signal generating compound capable of generating a detectable signal. The kit may also contain a control or calibrator which comprises a reagent which binds to the antigen.

The present invention also includes another type of kit for detecting antibodies such as auto-antibodies in a sample. The kit may comprise a) an anti-antibody specific for the antibody of interest, and b) Aβ(X-38 . . . 43) oligomer or derivative as defined herein. A control or calibrator comprising a reagent which binds to the Aβ(X-38 . . . 43) oligomer or derivative may also be included. More specifically, the kit may comprise a) an anti-antibody specific for the auto-antibody and b) a conjugate comprising the Aβ(X-38 . . . 43) oligomer or derivative, the conjugate being attached to a signal generating compound capable of generating a detectable signal. Again, the kit may also comprise a control or calibrator comprising a reagent which binds to the antigen.

The kit may also comprise one container such as a vial, bottle or strip, with each container with a pre-set solid phase, and other containers containing the respective conjugates. These kits may also contain vials or containers of other reagents needed for performing the assay, such as washing, processing and indicator reagents.

The Aβ(X-38 . . . 43) oligomers and derivatives of the invention are useful for providing agents that are capable of binding to the Aβ(X-38 . . . 43) oligomer or derivative. Such agents include, e.g., antibodies (hereinafter also referred to as anti-oligomer antibody), non-antibody binding molecules (such as affibodies, affilin molecules, AdNectins, Anticalins, DARPins, domain antibodies, evibodies, knotins, Kunitz-type domains, maxibodies, tetranectins, trans-bodies, and V(NAR)'s, as described, for instance, in the Handbook of Therapeutic Antibodies, ed. by Stefan Dübel, Volume II, Chapter 7, Wiley-VCH Verlag GmbH & Co. KGaA, Weinheim, 2007), aptamers or small-molecular weight compounds.

In one aspect, the invention thus relates to the use of the Aβ(X-38 . . . 43) oligomer or derivative for screening an agent that is capable of binding to the oligomer or derivative. Accordingly, the invention also relates to a method of identifying such agents, which method comprises the steps of: a) exposing one or more agents of interest to the oligomer or derivative described herein for a time and under conditions sufficient for the one or more agents to bind to the oligomer or derivative; and b) identifying those agents which bind to the oligomer or derivative.

In another aspect, the invention relates to the use of the Aβ(X-38 . . . 43) oligomer or derivative for enriching an agent that is capable of binding to the Aβ(X-38 . . . 43) oligomer or derivative in a preparation comprising said agent. Accordingly, the invention also relates to a method of enriching such an agent in a preparation comprising said agent, which method comprises the steps of: a) exposing to the Aβ(X-38 . . . 43) oligomer or derivative described herein the preparation comprising the agent that is capable of binding to the Aβ(X-38 . . . 43) oligomer or derivative for a time and under conditions sufficient for the agent to bind to the Aβ(X-38 . . . 43) oligomer or derivative; and b) obtaining the agent in enriched form. More particularly, the Aβ(X-38 . . . 43) oligomer or derivative can be immobilized (for instance on a resin), which allows the agent to be captured. Obtaining the agent in enriched form may then comprise desorbing the captured agent, preferably in such a way that desorbing the captured agent comprises contacting the captured agent with a high salt buffer or an acidic solution. This method can, for instance, be used for enriching auto-antibodies as described herein by subjecting commercial immunoglobulin preparations like IVIG or Octagam®(Octapharma Inc. Vienna, Austria) to this method. It is believed that these immunoglobulin preparations contain auto-antibodies to Aβ, and by treating subjects one raises the level of anti-Aβ antibodies in their body. A preparation that is enriched for said auto-antibodies would be expected to be more efficacious.

In a further aspect, the invention thus relates to the use of the Aβ(X-38 . . . 43) oligomer or derivative for providing an antibody that binds to the oligomer or derivative. Accordingly, the invention also relates to a method for providing an antibody that binds to the Aβ(X-38 . . . 43) oligomer or derivative as defined herein, which method comprises:

a) providing an antigen comprising the oligomer or derivative;
b) exposing an antibody repertoire or potential antibody repertoire to said antigen; and
c) selecting from said repertoire an antibody which binds to said oligomer or derivative thereof.

Here it is to be understood that a “potential antibody repertoire” refers to any library, collection, assembly or set of amino acid or corresponding nucleic acid sequences or to any generator of such a library, collection, assembly or set of amino acid sequences that can be used for producing an antibody repertoire in vivo or in vitro. In a preferred embodiment of the invention, the generator is the adaptive immune system of an animal, in particular the antigen-producing part of the immune system of a mammal which generates antibody diversity by a recombination process well known to those skilled in the art. In another preferred embodiment of the invention, the generator is a system for the spawning of random nucleic acid sequences which can then, by insertion into a suitable antibody framework, be used to produce an antibody repertoire in vitro.

In a preferred embodiment of the invention, the antibody repertoire or potential antibody repertoire is exposed to the antigen in vivo by immunizing an organism with said antigen. In another preferred embodiment of the invention, the potential antibody repertoire is a library of suitable nucleic acids which is exposed to the antibody by in vitro affinity screening as described in the art, e.g. a phage display and panning system.

In another aspect, the invention also provides antibodies that bind to the Aβ(X-38 . . . 43) oligomer or derivative as defined herein.

In a preferred embodiment of the invention, the antibody is obtainable by a method comprising selecting the antibody from a repertoire or potential repertoire as described herein.

According to a particularly preferred embodiment, the present invention provides oligomer-specific antibodies. These include in particular antibodies having a comparatively smaller affinity for both the monomeric and fibrillomeric forms of Aβ peptide than for the Aβ(X-38 . . . 43) oligomer or derivative of the invention. In certain embodiments, an antibody is said to specifically bind an antigen when it preferentially recognizes its target antigen in a complex mixture of proteins and/or macromolecules.

In a preferred embodiment of the invention, the affinity of the antibody to the oligomer or derivative is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to a monomeric Aβ(1-42).

In a preferred embodiment of the invention, the affinity of the antibody to the oligomer or derivative is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to a monomeric Aβ(1-40).

Expediently, the antibody of the present invention binds to one or, more preferably, both monomers with low affinity, most preferably with a K_{D} of 1×10⁻⁸ M or smaller affinity, e.g. with a K_{D} of 3×10⁻⁸ M or smaller affinity, with a K_{D} of 1×10⁻⁷ M or smaller affinity, e.g. with a K_{D} of 3×10⁻⁷ M or smaller affinity, or with a K_{D} of 1×10⁻⁸ M or smaller affinity, e.g. with a K_{D} of 3×10⁻⁵ M or smaller affinity, or with a K_{D} of 1×10⁻⁵ M or smaller affinity.

In a preferred embodiment of the invention, the affinity of the antibody to the oligomer or derivative is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to a fibrillomeric Aβ(1-42).

In a preferred embodiment of the invention, the affinity of the antibody to the oligomer or derivative is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to a fibrillomeric Aβ(1-40).

Expediently, the antibody of the present invention binds to one or, more preferably, both fibrils with low affinity, most preferably with a K_{D} of 1×10⁻⁸ M or smaller affinity, e.g. with a K_{D} of 3×10⁻⁸ M or smaller affinity, with a K_{D} of 1×10⁻⁷ M or smaller affinity, e.g. with a K_{D} of 3×10⁻⁷ M or smaller affinity, or with a K_{D} of 1×10⁻⁶ M or smaller affinity, e.g. with a K_{D} of 3×10⁻⁵ M or smaller affinity, or with a K_{D} of 1×10⁻⁵ M or smaller affinity.

The antibodies of the present invention are preferably isolated antibodies. An “isolated antibody” means an antibody having the binding affinities as described above and which is essentially free of other antibodies having different binding affinities. The term “essentially free” here refers to an antibody preparation in which at least 95% of the antibodies, preferably at least 98% of the antibodies and more preferably at least 99% of the antibodies have the desired binding affinity. Moreover, an isolated antibody may be substantially free of other cellular material and/or chemicals.

The isolated antibodies of the present invention include monoclonal antibodies. A “monoclonal antibody” as used herein is intended to refer to a preparation of antibody molecules, antibodies which share a common heavy chain and common light chain amino acid sequence, in contrast with “polyclonal” antibody preparations which contain a mixture of antibodies of different amino acid sequence. Monoclonal antibodies can be generated by several novel technologies like phage, bacteria, yeast or ribosomal display, as well as by classical methods exemplified by hybridoma-derived antibodies (e.g., an antibody secreted by a hybridoma prepared by hybridoma technology, such as the standard Kohler and Milstein hybridoma methodology ((1975) Nature 256:495-497). Thus, a non-hybridoma-derived antibody with uniform sequence is still referred to as a monoclonal antibody herein although it may have been obtained by non-classical methodologies, and the term “monoclonal” is not restricted to hybridoma-derived antibodies but used to refer to all antibodies derived from one nucleic acid clone.

Thus, the monoclonal antibodies of the present invention include recombinant antibodies. The term “recombinant” herein refers to any artificial combination of two otherwise separated segments of sequence, e.g., by chemical synthesis or by the manipulation of isolated segments of nucleic acids by genetic engineering techniques. In particular, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes (see, for example, Taylor, L. D., et al. (1992) Nucl. Acids Res. 20:6287-6295); or antibodies which are produced, expressed, generated or isolated in any other way in which particular immunoglobulin gene sequences (such as human immunoglobulin gene sequences) are assembled with other DNA sequences. Recombinant antibodies include, for example, chimeric, CDR graft and humanized antibodies. The person skilled in the art will be aware that expression of a conventional hybridoma-derived monoclonal antibody in a heterologous system will require the generation of a recombinant antibody even if the amino acid sequence of the resulting antibody protein is not changed or intended to be changed.

In a particular embodiment of the invention, the antibody is a humanized antibody.

According to a multiplicity of embodiments, the antibody may comprise an amino acid sequence derived entirely from a single species, such as a human antibody or a mouse antibody. According to other embodiments, the antibody may be a chimeric antibody or a CDR graft antibody or another form of a humanized antibody.

The term “antibody” is intended to refer to immunoglobulin molecules consisting of 4 polypeptide chains, two heavy (H) chains and two light (L) chains. The chains are usually linked to one another via disulfide bonds. Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

The term “antigen-binding moiety” of an antibody (or simply “antibody moiety”) refers to one or more fragments of an antibody of the invention, said fragment(s) still having the binding affinities as defined above. Fragments of a complete antibody have been shown to be able to carry out the antigen-binding function of an antibody. In accordance with the term “antigen-binding moiety” of an antibody, examples of binding fragments include (i) an Fab fragment, i.e. a monovalent fragment composed of the VL, VH, CL and CH1 domains; (ii) an

F(ab')₂ fragment, i.e. a bivalent fragment comprising two Fab fragments linked to one another in the hinge region via a disulfide bridge; (iii) an Fd fragment composed of the VH and CH1 domains; (iv) an Fv fragment composed of the FL and VH domains of a single arm of an antibody; (v) a dAb fragment (Ward et al., (1989) Nature 341:544-546) consisting of a VH domain or of VH, CH1, CH2, DH3, or VH, CH2, CH3; and (vi) an isolated complementarity-determining region (CDR). Although the two domains of the Fv fragment, namely VL and VH, are encoded by separate genes, they may further be linked to one another using a synthetic linker, e.g. a poly-G₄S amino acid sequence, and recombinant methods, making it possible to prepare them as a single protein chain in which the VL and VH regions combine in order to form monovalent molecules (known as single chain Fv (ScFv); see, for example, Bird et al. (1988) Science 242:423-426; and Huston et al. (1988) Proc. Natl. Acad. Sci. USA 85:5879-5883). The term “antigen-binding moiety” of an antibody is also intended to comprise such single chain antibodies. Other forms of single chain antibodies such as “diabodies” are likewise included here. Diabodies are bivalent, bispecific antibodies in which VH and VL domains are expressed on a single polypeptide chain, but using a linker which is too short for the two domains being able to combine on the same chain, thereby forcing said domains to pair with complementary domains of a different chain and to form two antigen-binding sites (see, for example, Holliger, P., et al. (1993)Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art.

Furthermore, an antibody of the present invention or antigen-binding moiety thereof may be part of a larger immunoadhesion molecule formed by covalent or noncovalent association of said antibody or antibody-binding moiety with one or more further proteins or peptides. Relevant to such immunoadhesion molecules are the use of the streptavidin core region in order to prepare a tetrameric scFv molecule (Kipriyanov, S. M., et al. (1995) Human Antibodies and Hybridomas 6:93-101) and the use of a cystein residue, a marker peptide and a C-terminal polyhistidinyl, e.g. hexahistidinyl, tag in order to produce bivalent and biotinylated scFv molecules (Kipriyanov, S. M., et al. (1994) Mol. Immunol. 31:1047-1058).

The term “human antibody” refers to antibodies whose variable and constant regions correspond to or are derived from immunoglobulin sequences of the human germ line, as described, for example, by Kabat et al. (see Kabat, et al. (1991) Sequences of Proteins of Immunological Interest, Fifth Edition, U.S. Department of Health and Human Services, NIH Publication No. 91-3242). However, the human antibodies of the invention may contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies of the invention have variable regions and may also contain constant regions derived from immunoglobulin sequences of the human germ line (see Kabat, E. A., et al. (1991) Sequences of Proteins of Immunological Interest, Fifth Edition, U.S. Department of Health and Human Services, NIH Publication No. 91-3242). According to particular embodiments, however, such recombinant human antibodies are subjected to in-vitro mutagenesis (or to a somatic in-vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both.

Preferably, mutagenesis leads to an affinity to the target which is greater, and/or an affinity to non-target structures which is smaller than that of the parent antibody.

The term “chimeric antibody” refers to antibodies which contain sequences for the variable region of the heavy and light chains from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies which contain sequences of the variable region of heavy and light chains from a nonhuman species (e.g. mouse, rat, rabbit, chicken, camelid, sheep or goat) but in which at least one part of the VH and/or VL sequence has been altered in order to be more “human-like”, i.e. to be more similar to variable sequences of the human germ line. One type of a humanized antibody is a CDR graft antibody in which human CDR sequences have been inserted into nonhuman VH and VL sequences to replace the corresponding nonhuman CDR sequences.

Methods of producing antibodies of the invention are described below. A distinction is made here between in-vivo approaches, in-vitro approaches or a combination of both.

Some methods of producing antibodies of the invention are described below. A distinction is made here between in-vivo approaches, in-vitro approaches or a combination of both.

### In-Vivo Approaches

Depending on the type of the desired antibody, various host animals may be used for in-vivo immunization. A host expressing itself an endogenous version of the antigen of interest may be used. Alternatively, it is possible to use a host which has been made deficient in an endogenous version of the antigen of interest. For example, mice which had been made deficient in a particular endogenous protein via homologous recombination at the corresponding endogenous gene (i.e. knockout mice) have been shown to generate a humoral response to the protein with which they have been immunized and therefore to be able to be used for production of high-affinity monoclonal antibodies to the protein (see, for example, Roes, J. et al. (1995) J. Immunol. Methods 183:231-237; Lunn, M. P. et al. (2000) J. Neurochem. 75:404-412).

A multiplicity of nonhuman mammals are suitable hosts for antibody production in order to produce nonhuman antibodies of the invention. They include mice, rats, chickens, camelids, rabbits, sheep and goats (and knockout versions thereof), although preference is given to mice for the production of hybridomas. Furthermore, a nonhuman host animal expressing a human antibody repertoire may be used for producing essentially human antibodies to a human antigen with dual specificity. Nonhuman animals of this kind include transgenic animals (e.g. mice) bearing human immunoglobulin transgenes (chimeric hu-PBMC SCID mice) and human/mouse irradiation chimeras which are described in more detail below.

According to one embodiment, the animal immunized with oligomer or derivative is a nonhuman mammal, preferably a mouse, which is transgenic due to human immunoglobulin genes so that said nonhuman mammal makes human antibodies upon antigenic stimulation. Typically, immunoglobulin transgenes for heavy and light chains with human germ line configuration are introduced into such animals which have been altered such that their endogenous heavy and light chain loci are inactive. If such animals are stimulated with antigen (e.g. with a human antigen), antibodies derived from the human immunoglobulin sequences (human antibodies) are produced. It is possible to make from the lymphocytes of such animals human monoclonal antibodies by means of standardized hybridoma technology. For a further description of transgenic mice with human immunoglobulins and their use in the production of human antibodies, see, for example, U.S. Pat. No. 5,939,598, WO 96/33735, WO 96/34096, WO 98/24893 and WO 99/53049 (Abgenix Inc.), and U.S. Pat. No. 5,545,806, U.S. Pat. No. 5,569,825, U.S. Pat. No. 5,625,126, U.S. Pat. No. 5,633,425, U.S. Pat. No. 5,661,016, U.S. Pat. No. 5,770,429, U.S. Pat. No. 5,814,318, U.S. Pat. No. 5,877,397 and WO 99/45962 (Genpharm Inc.); see also MacQuitty, J. J. and Kay, R. M. (1992) Science 257:1188; Taylor, L. D. et al. (1992) Nucleic Acids Res. 20:6287-6295; Lonberg, N. et al. (1994) Nature 368:856-859; Lonberg, N. and Huszar, D. (1995) Int. Rev. Immunol. 13:65-93; Harding, F. A. and Lonberg, N. (1995) Ann. N.Y. Acad. Sci. 764:536-546; Fishwild, D. M. et al. (1996) Nature Biotechnology 14:845-851; Mendez, M. J. et al. (1997) Nature Genetics 15:146-156; Green, L. L. and Jakobovits, A. (1998) J. Exp. Med. 188:483-495; Green, L. L. (1999) J. Immunol. Methods 231:11-23; Yang, X. D. et al. (1999) J. Leukoc. Biol. 66:401-410; Gallo, M. L. et al. (2000) Eur. J. Immunol. 30:534-540.

According to another embodiment, the animal which is immunized with the oligomer or derivative may be a mouse with severe combined immunodeficiency (SCID), which has been reconstituted with human peripheral mononuclear blood cells or lymphoid cells or precursors thereof. Such mice which are referred to as chimeric hu-PBMC SCID mice produce human immunoglobulin responses upon antigenic stimulation, as has been proved. For a further description of these mice and of their use for generating antibodies, see, for example, Leader, K. A. et al. (1992) Immunology 76:229-234; Bombil, F. et al. (1996) Immunobiol. 195:360-375; Murphy, W. J. et al. (1996) Semin. Immunol. 8:233-241; Herz, U. et al. (1997) Int. Arch. Allergy Immunol. 113:150-152; Albert, S. E. et al. (1997) J. Immunol. 159:1393-1403; Nguyen, H. et al. (1997) Microbiol. Immunol. 41:901-907; Arai, K. et al. (1998) J. Immunol. Methods 217:79-85; Yoshinari, K. and Arai, K. (1998) Hybridoma 17:41-45; Hutchins, W. A. et al. (1999) Hybridoma 18:121-129; Murphy, W. J. et al. (1999) Clin. Immunol. 90:22-27; Smithson, S. L. et al. (1999) Mol. Immunol. 36:113-124; Chamat, S. et al. (1999) J. Infect. Diseases 180:268-277; and Heard, C. et al. (1999) Molec. Med. 5:35-45.

According to another embodiment, the animal which is immunized with the oligomer or derivative is a mouse which has been treated with a lethal dose of total body irradiation, then protected from radiation with bone marrow cells from mice with severe combined immunodeficiency (SCID) and subsequently transplanted with functional human lymphocytes. This type of chimera, referred to as the Trimera system, is used in order to produce human monoclonal antibodies by immunizing said mice with the antigen of interest and then producing monoclonal antibodies by using standardized hybridoma technology. For a further description of these mice and of their use for generating antibodies, see, for example, Eren, R. et al. (1998) Immunology 93:154-161; Reisner, Y. and Dagan, S. (1998) Trends Biotechnol. 16:242-246; Ilan, E. et al. (1999) Hepatology 29:553-562; and Bocher, W. O. et al. (1999) Immunology 96:634-641.

Starting from the in-vivo generated antibody-producing cells, monoclonal antibodies may be produced by means of standardized techniques such as the hybridoma technique originally described by Kohler and Milstein (1975, Nature 256:495-497) (see also Brown et al. (1981) J. Immunol. 127:539-46; Brown et al. (1980) J Biol Chem 255:4980-83; Yeh et al. (1976) PNAS 76:2927-31; and Yeh et al. (1982) Int. J. Cancer 29:269-75). The technology of producing monoclonal antibody hybridomas is sufficiently known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet., 3:231-36). Briefly, an immortalized cell line (typically a myeloma) is fused with lymphocytes (typically splenocytes or lymph node cells or peripheral blood lymphocytes) of a mammal immunized with the oligomer or derivative of the invention, and the culture supernatants of the resulting hybridoma cells are screened in order to identify a hybridoma which produces a monoclonal antibody of the present invention. Any of the many well known protocols for fusing lymphocytes and immortalized cell lines can be applied for this purpose (see also G. Galfre et al. (1977) Nature 266:550-52; Gefter et al. Somatic Cell Genet., cited supra; Lerner, Yale J. Biol. Med., cited supra; Kenneth, Monoclonal Antibodies, cited supra). Moreover, the skilled worker will appreciate that there are diverse variations of such methods, which are likewise useful. Typically, the immortalized cell line (e.g. a myeloma cell line) is derived from the same mammalian species as the lymphocytes. For example, murine hybridomas may be established by fusing lymphocytes from a mouse immunized with an immunogenic preparation of the invention with an immortalized mouse cell line. Preferred immortalized cell lines are mouse myeloma cell lines which are sensitive to culture medium containing hypoxanthine, aminopterine and thymidine (HAT medium). Any of a number of myeloma cell lines may be used by default as fusion partner, for example the P3-NS1/1-Ag4-1, P3-x63-Ag8.653 or Sp2/O—Ag14 myeloma lines. These myeloma cell lines are available from the American Type Culture Collection (ATCC), Rockville, Md. Typically, HAT-sensitive mouse myeloma cells are fused to mouse splenocytes using polyethylene glycol (PEG). Hybridoma cells resulting from the fusion are then selected using HAT medium, thereby killing unfused and unproductively fused myeloma cells (unfused splenocytes die after several days because they are not transformed). Hybridoma cells producing monoclonal antibodies of the invention are identified by screening the hybridoma culture supernatants for such antibodies, for example by using a dot blot assay in order to select those antibodies which have the binding affinities as defined herein.

Likewise, said hybridoma can be used as a source of nucleic acid encoding light and/or heavy chains in order to recombinantly produce antibodies of the present invention, as is described below in further detail.

### In-Vitro Approaches

As an alternative to producing antibodies of the invention by immunization and selection, antibodies of the invention may be identified and isolated by screening recombinant combinatorial immunoglobulin libraries with the oligomer or derivative to thereby isolate immunoglobulin library members which have the required binding affinity. Kits for generating and screening display libraries are commercially available (e.g. the Pharmacia Recombinant NaH₂PO₄, 140 mM NaCl, 1% SDS, pH 7.4) and 100 μl of the mixture immediately analyzed by SEC.

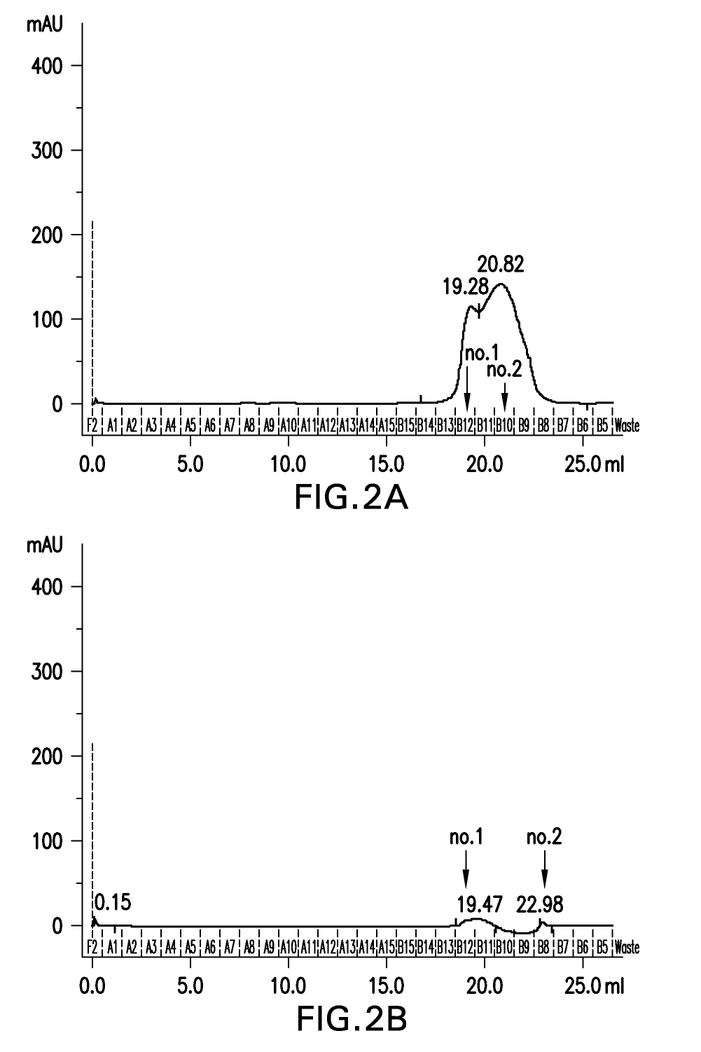

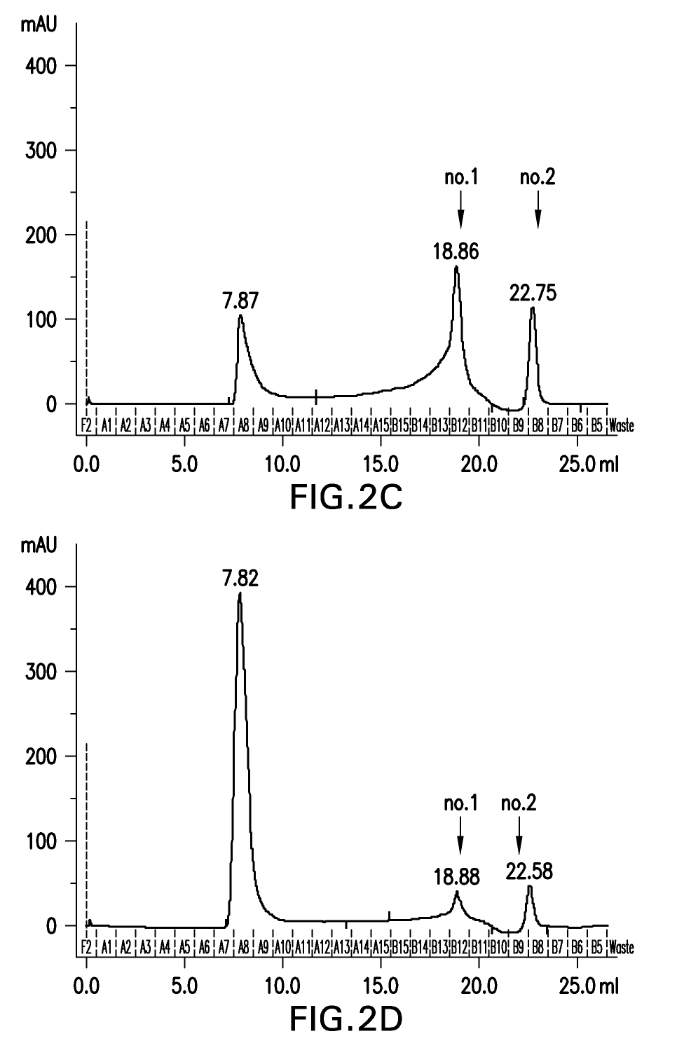

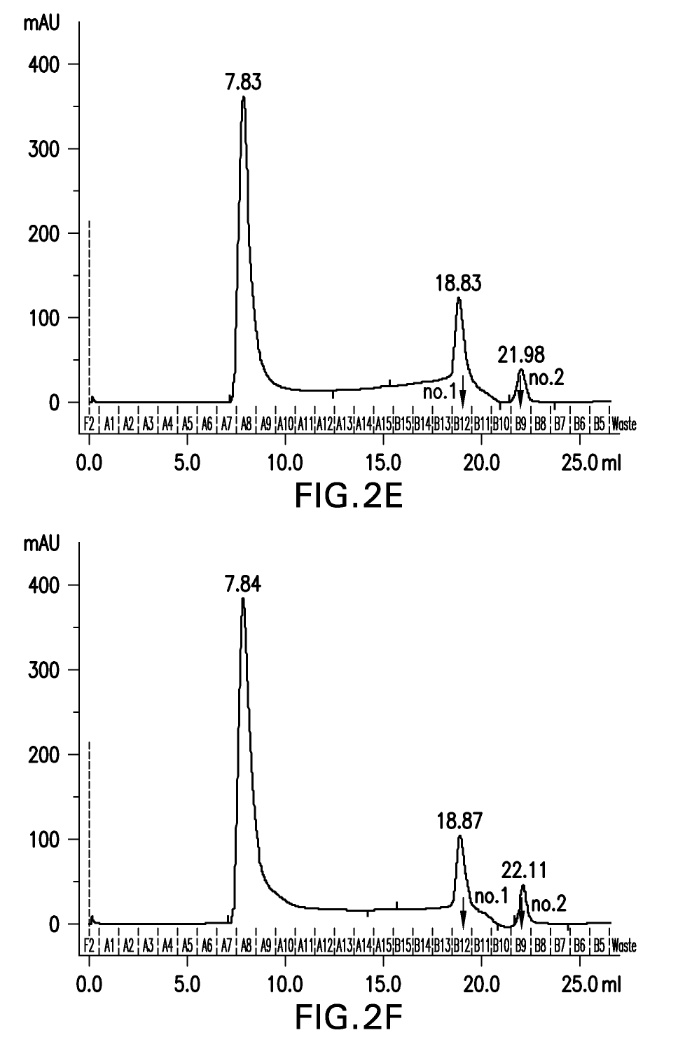

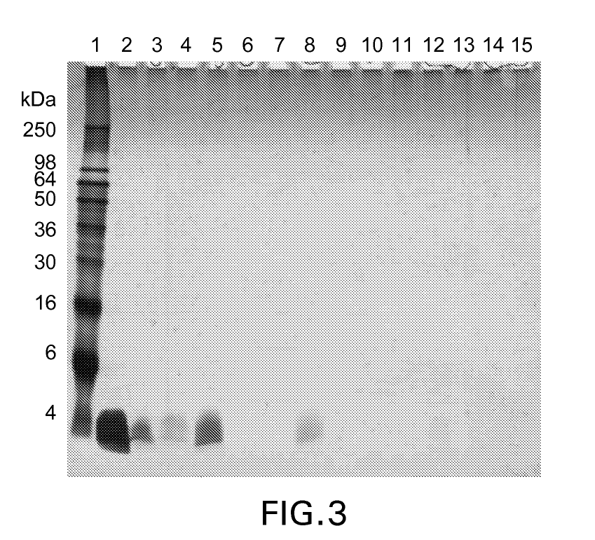

The resulting size exclusion chromatogram (FIG. 2E) showed peaks at ˜8 ml, ˜19 ml and ˜21 ml elution volume. Peak hights were similar to those observed in example 13d. SDS-PAGE analysis (FIG. 3, lanes 12 and 13) revealed that the peak at ˜8 ml contained most of the peptide. The peaks at ˜19 ml and ˜21 ml elution volume did not contain any detectable Aβ(20-42) peptide.

This indicates that the amount of Aβ(20-42) HMW oligomer formed in this example was comparable to that formed in example 13d. The Aβ(20-42) HMW oligomer formation was complete.

f) Aβ(20-42) peptide sample in buffer 0.25% SDS for 2 h at 37° C. with open tube lid followed for 20 h at room temperature with close tube lid: 250 μL buffer composed of 5 mM NaH₂PO₄, 35 mM NaCl, 0.25% SDS, pH 7.4, were added to 12.5 μl of the Aβ(20-42) HFIP solution (20 mg/mL) and the sample was incubated at 37° C. under shaking for 10 min. The sample was centrifuged at room temperature for 10 min at 10,000×g. Supernatant was incubated for 2 h with open tube lid at 850 rpm in an orbital shaker at 37° C. Subsequently the lid was closed and the sample further incubated for 20 h at room temperature. 75 μl of the sample were mixed with 75 μl of the SEC mobile phase (20 mM NaH₂PO₄, 140 mM NaCl, 1% SDS, pH 7.4) and 100 μl of the mixture immediately analyzed by SEC.

The resulting size exclusion chromatogram (FIG. 2F) showed peaks at ˜8 ml, ˜19 ml and ˜21 ml elution volume. Peak heights were similar to those observed in example 13d and 13e. SDS-PAGE analysis (FIG. 3, lanes 14 and 15) revealed that the peak at ˜8 ml contained most of the peptide. The peaks at ˜19 ml and ˜21 ml elution volume did not contain any detectable Aβ(20-42) peptide.

This indicates that in this example no additional Aβ(20-42) HMW oligomer was formed when compared to the amount of Aβ(20-42) HMW oligomer formed in example 13d or 13e. The Aβ(20-42) HMW oligomer formation was complete.

SDS-PAGE analysis of the size exclusion chromatography peaks SDS-PAGE:

Phage Antibody System, catalog No. 27-9400-01; and the Stratagene SurfZAP® Phage Display Kit, catalog No. 240612). In many embodiments, the display library is an scFv library or an Fab library. The phage display technique for screening recombinant antibody libraries has been adequately described. Examples of methods and compounds which can be used particularly advantageously for generating and screening antibody display libraries can be found, for example, in McCafferty et al. WO 92/01047, U.S. Pat. No. 5,969,108 and EP 589 877 (describes in particular scFv display), Ladner et al. U.S. Pat. No. 5,223,409, U.S. Pat. No. 5,403,484, U.S. Pat. No. 5,571,698, U.S. Pat. No. 5,837,500 and EP 436 597 (describes pill fusion, for example); Dower et al. WO 91/17271, U.S. Pat. No. 5,427,908, U.S. Pat. No. 5,580,717 and EP 527 839 (describes in particular Fab display); Winter et al. International Publication WO 92/20791 and EP 368,684 (describes in particular the cloning of sequences for variable immunoglobulin domains); Griffiths et al. U.S. Pat. No. 5,885,793 and EP 589 877 (describes in particular isolation of human antibodies to human antigens by using recombinant libraries); Garrard et al. WO 92/09690 (describes in particular phage expression techniques); Knappik et al. WO 97/08320 (describes the human recombinant antibody library HuCal); Salfeld et al. WO 97/29131, (describes production of a recombinant human antibody to a human antigen (human tumor necrosis factor alpha) and also in-vitro affinity maturation of the recombinant antibody) and Salfeld et al. U.S. Provisional Application No. 60/126,603 and the patent applications based hereupon (likewise describes production of recombinant human antibodies to human antigen (human interleukin-12), and also in-vitro affinity maturation of the recombinant antibody).

Further descriptions of screenings of recombinant antibody libraries can be found in scientific publications such as Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clarkson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; Barbas et al. (1991) PNAS 88:7978-7982; McCafferty et al. Nature (1990) 348:552-554; and Knappik et al. (2000) J. Mol. Biol. 296:57-86.

As an alternative to using bacteriophage display systems, recombinant antibody libraries may be expressed on the surface of yeast cells or of bacterial cells. WO 99/36569 describes methods of preparing and screening libraries expressed on the surface of yeast cells. WO 98/49286 describes in more detail methods of preparing and screening libraries expressed on the surface of bacterial cells.

In all in vitro approaches, a selection process for enriching recombinant antibodies with the desired properties form an integral part of the process, which is generally referred to as “panning” and often takes the form of affinity chromatography over columns to whose matrix the target structure has been attached. Promising candidate molecules are then subjected to individual determination of their absolute and/or relative affinities, preferably by means of a standardized dot blot assay, as described above.

As may be appreciated by skilled workers, such in vitro methods for selection and enrichment may also be applied towards obtaining non-immunoglobulin related antigen-binding moieties.

Once an antibody of interest of a combinatorial library has been identified and sufficiently characterized, the DNA sequences encoding the light and heavy chains of said antibody are isolated by means of standardized molecular-biological techniques, for example by means of PCR amplification of DNA from the display package (e.g. the phage) which has been isolated during library screening. Nucleotide sequences of genes for light and heavy antibody chains, which may be used for preparing PCR primers, are known to the skilled worker. A multiplicity of such sequences are described, for example, in Kabat, E. A., et al. (1991) Sequences of Proteins of Immunological Interest, Fifth Edition, U.S. Department of Health and Human Services, NIH Publication No. 91-3242 and in the database of sequences of the human germ line VBASE.

An antibody or antibody-binding moiety of the invention may be produced by recombinantly expressing the genes for light and heavy immunoglobulin chains in a host cell. In order to recombinantly express an antibody, a host cell is transfected with one or more recombinant expression vectors carrying DNA fragments encoding the light and heavy immunoglobulin chains of said antibody, thereby expressing the light and heavy chains in the host cell and secreting them preferably into the medium in which said host cells are cultured. The antibodies can be isolated from this medium. Standardized recombinant DNA methods are used in order to obtain genes for heavy and light antibody chains, to insert said genes into recombinant expression vectors and to introduce said vectors into host cells. Methods of this kind are described, for example, in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

Once DNA fragments encoding VH and VL segments of the antibody of interest have been obtained, said DNA fragments may be further manipulated using standardized recombinant DNA techniques, for example in order to convert the genes for variable regions to genes for full length antibody chains, to genes for Fab fragments or to an scFv gene. These manipulations comprise linking a VL- or VH-encoding DNA fragment operatively to another DNA fragment encoding another protein, for example a constant antibody region or a flexible linker. The term “operatively linked” is to be understood here as meaning that the two DNA fragments are linked in such a way that the amino acid sequences encoded by said two DNA fragments remain in frame.

The isolated DNA encoding the VH region may be converted to a gene for a full length heavy chain by operatively linking the VH-region encoding DNA with another DNA molecule encoding heavy chain constant regions (CH1, CH2 and CH3). The sequences of human heavy chain constant region genes are well known (see, for example, Kabat, E. A., et al. (1991) Sequences of Proteins of Immunological Interest, Fifth Edition, U.S. Department of Health and Human Services, NIH Publication No. 91-3242), and DNA fragments spanning said regions may be obtained by means of standardized PCR amplification. The heavy chain constant region may be a constant region from IgG1, IgG2, IgG3, IgG4, IgM, IgA, IgE or IgD, with preference being given to a constant region from IgG, in particular IgG1 or IgG4. To obtain a gene for a heavy chain Fab fragment, the VH-encoding DNA may be operatively linked to another DNA molecule encoding merely the heavy chain constant region CH₁.

The isolated DNA encoding the VL region may be converted to a gene for a full length light chain (and a gene for an Fab light chain) by operatively linking the VL-encoding DNA to another DNA molecule encoding the light chain constant region CL. The sequences of genes of the constant region of human light chain are well known (see Kabat, E. A., et al. (1991) Sequences of Proteins of Immunological Interest, Fifth Edition, U.S. Department of Health and Human Services, NIH Publication No. 91-3242), and DNA fragments spanning said regions may be obtained by means of standardized PCR amplification. The light chain constant region may be a constant kappa or lambda region, a constant kappa region being preferred.

In order to generate an scFv gene, the VH- and VL-encoding DNA fragments may be operatively linked to another fragment encoding a flexible linker, for example the amino acid sequence (Gly₄-Ser)₃ so that the VH and VL sequences are expressed as a continuous single-chain protein, with the VL and VH regions being linked to one another via said flexible linker (see Bird et al. (1988) Science 242:423-426; Huston et al. (1988) Proc. Natl. Acad. Sci. USA 85:5879-5883; McCafferty et al., Nature (1990) 348:552-554).

Single domain VH and VL having the binding affinities as described above may be isolated from single domain libraries by the above-described methods. Two VH single-domain chains (with or without CH1) or two VL chains or a pair of one VH chain and one VL chain with the desired binding affinity may be useful as described herein for the antibodies of the invention.

In order to express the recombinant antibodies or antibody moieties of the invention, the DNAs encoding partial or full length light and heavy chains may be inserted into expression vectors so as to operatively link the genes to appropriate transcriptional and translational control sequences. In this context, the term “operatively linked” is to be understood as meaning that an antibody gene is ligated in a vector in such a way that transcriptional and translational control sequences within the vector fulfill their intended function of regulating transcription and translation of said antibody gene.

Expediently, the expression vector and the expression control sequences are chosen so as to be compatible with the expression host cell used. The gene for the antibody light chain and the gene for the antibody heavy chain may be inserted into separate vectors or both genes are inserted into the same expression vector, this being the usual case. The antibody genes are inserted into the expression vector by means of standardized methods (for example by ligation of complementary restriction cleavage sites on the antibody gene fragment and the vector, or by ligation of blunt ends, if no restriction cleavage sites are present). The expression vector may already carry sequences for antibody constant regions prior to insertion of the sequences for the light and heavy chains. For example, one approach is to convert the VH and VL sequences to full length antibody genes by inserting them into expression vectors already encoding the heavy and, respectively, light chain constant regions, thereby operatively linking the VH segment to the CH segment(s) within the vector and also operatively linking the VL segment to the CL segment within the vector.

Additionally or alternatively, the recombinant expression vector may encode a signal peptide which facilitates secretion of the antibody chain from the host cell. The gene for said antibody chain may be cloned into the vector, thereby linking the signal peptide in frame to the N terminus of the gene for the antibody chain. The signal peptide may be an immuno-globulin signal peptide or a heterologous signal peptide (i.e. a signal peptide from a non-immunoglobulin protein). In addition to the genes for the antibody chain, the expression vectors of the invention may have regulatory sequences controlling expression of the genes for the antibody chain in a host cell.

The term “regulatory sequence” is intended to include promoters, enhancers and further expression control elements (e.g. polyadenylation signals) which control transcription or translation of the genes for the antibody chain. Regulatory sequences of this kind are described, for example, in Goeddel; Gene Expression Technology: Methods in Enzymology 185, Academic Press, San Diego, Calif. (1990). The skilled worker will appreciate that the expression vector design which includes selection of regulatory sequences may depend on factors such as the choice of the host cell to be transformed, the desired strength of expression of the protein, etc. Preferred regulatory sequences for expression in mammalian host cells include viral elements resulting in strong and constitutive protein expression in mammalian cells, such as promoters and/or enhancers derived from cytomegalovirus (CMV) (such as the CMV promoter/enhancer), simian virus 40 (SV40) (such as the SV40 promoter/enhancer), adenovirus (e.g. the adenovirus major late promoter (AdMLP)) and polyoma. For a further description of viral regulatory elements and sequences thereof, see, for example, U.S. Pat. No. 5,168,062 to Stinski, U.S. Pat. No. 4,510,245 to Bell et al. and U.S. Pat. No. 4,968,615 to Schaffner et al.

Apart from the genes for the antibody chain and the regulatory sequences, the recombinant expression vectors of the invention may have additional sequences such as those which regulate replication of the vector in host cells (e.g. origins of replication) and selectable marker genes. The selectable marker genes facilitate the selection of host cells into which the vector has been introduced (see, for example, U.S. Pat. Nos. 4,399,216, 4,634,665 and 5,179,017, all to Axel et al.). For example, it is common for the selectable marker gene to render a host cell into which the vector has been inserted resistant to cytotoxic drugs such as G418, hygromycin or methotrexate. Preferred selectable marker genes include the gene for dihydrofolate reductase (DHFR) (for use in dhfr⁻ host cells with methotrexate selection/amplification) and the neo gene (for G418 selection).

For expression of the light and heavy chains, the expression vector(s) encoding said heavy and light chains is(are) transfected into a host cell by means of standardized techniques. The various forms of the term “transfection” are intended to comprise a multiplicity of techniques customarily used for introducing exogenous DNA into a prokaryotic or eukaryotic host cell, for example electroporation, calcium phosphate precipitation, DEAE-dextran transfection, and the like. Although it is theoretically possible to express the antibodies of the invention either in prokaryotic or eukaryotic host cells, preference is given to expressing the antibodies in eukaryotic cells and, in particular, in mammalian host cells, since the probability of a correctly folded and immunologically active antibody being assembled and secreted is higher in such eukaryotic cells and in particular mammalian cells than in prokaryotic cells. Prokaryotic expression of antibody genes has been reported as being ineffective for production of high yields of active antibody (Boss, M. A. and Wood, C. R. (1985) Immunology Today 6:12-13).

Preferred mammalian host cells for expressing recombinant antibodies of the invention include CHO cells (including dhfr CHO cells described in Urlaub and Chasin, (1980)Proc. Natl. Acad. Sci. USA 77:4216-4220, which are used together with a DHFR-selectable marker, as described, for example, in R. J. Kaufman and P. A. Sharp (1982) Mol. Biol. 159:601-621), NSO myeloma cells, COS cells and SP2 cells. When introducing recombinant expression vectors encoding the antibody genes into mammalian host cells, the antibodies are produced by culturing the host cells until the antibody is expressed in said host cells or, preferably, the antibody is secreted into the culture medium in which the host cells grow. The antibodies may then be isolated from the culture medium by using standardized protein purification methods.

It is likewise possible to use host cells in order to produce moieties of intact antibodies, such as Fab fragments or scFv molecules. Variations of the above-described procedure are of course included in the invention. For example, it may be desirable to transfect a host cell with DNA encoding either the light chain or the heavy chain (but not both) of an antibody of the invention. If either light or heavy chains are present which are not required for binding of the antigen of interest, then the DNA encoding either such a light or such a heavy chain or both may be removed partially or completely by means of recombinant DNA technology. Molecules expressed by such truncated DNA molecules are likewise included in the antibodies of the invention. In addition, it is possible to produce bifunctional antibodies in which a heavy chain and a light chain are an antibody of the invention and the other heavy chain and the other light chain have specificity for an antigen different from the antigen of interest, by crosslinking an antibody of the invention to a second antibody by means of standardized chemical methods.

In a preferred system for recombinant expression of an antibody of the invention or an antigen-binding moiety thereof, a recombinant expression vector encoding both the antibody heavy chain and the antibody light chain is introduced into dhfr⁻ CHO cells by means of calcium phosphate-mediated transfection. Within the recombinant expression vector, the genes for the heavy and light antibody chains are in each case operatively linked to regulatory CMV enhancer/AdMLP-promoter elements in order to effect strong transcription of said genes. The recombinant expression vector also carries a DHFR gene which can be used for selecting dhfr⁻ CHO cells transfected with the vector by using methotrexate selection/amplification. The selected transformed host cells are cultured so that the heavy and light antibody chains are expressed, and intact antibody is isolated from the culture medium. Standardized molecular-biological techniques are used in order to prepare the recombinant expression vector, to transfect the host cells, to select the transformants, to culture said host cells, and to obtain the antibody from the culture medium. Thus the invention also provides a method of synthesizing a recombinant antibody of the invention by culturing a host cell of the invention in a suitable culture medium until a recombinant antibody of the invention has been synthesized. The method may furthermore comprise isolating said recombinant antibody from said culture medium.

As an alternative to screening recombinant antibody libraries by phage display, other methods known to the skilled worker may be used for screening large combinatorial libraries to identify the antibodies of the invention. Basically, any expression system in which a close physical linkage between a nucleic acid and the antibody encoded thereby is established and may be used to select a suitable nucleic acid sequence by virtue of the properties of the antibody it encodes may be employed.

In one type of an alternative expression system, the recombinant antibody library is expressed in the form of RNA-protein fusions, as described in WO 98/31700 to Szostak and Roberts, and in Roberts, R. W. and Szostak, J. W. (1997) Proc. Natl. Acad. Sci. USA 94:12297-12302. In this system, in-vitro translation of synthetic mRNAs carrying on their 3′ end puromycin, a peptidyl acceptor antibiotic, generates a covalent fusion of an mRNA and the peptide or protein encoded by it. Thus a specific mRNA of a complex mixture of mRNAs (e.g. a combinatorial library) may be concentrated on the basis of the properties of the encoded peptide or protein (e.g. of the antibody or a moiety thereof), such as binding of said antibody or said moiety thereof to the oligomer or derivative. Nucleic acid sequences which encode antibodies or moieties thereof and which are obtained by screening of such libraries may be expressed by recombinant means in the above-described manner (e.g. in mammalian host cells) and may, in addition, be subjected to further affinity maturation by either screening in further rounds mRNA-peptide fusions, introducing mutations into the originally selected sequence(s), or using other methods of in-vitro affinity maturation of recombinant antibodies in the above-described manner.

### Combinations of In-Vivo and In-Vitro Approaches

The antibodies of the invention may likewise be produced by using a combination of in-vivo and in-vitro approaches such as methods in which the oligomer or derivative is first allowed to act on an antibody repertoire in a host animal in vivo to stimulate production of oligomer- or derivative-binding antibodies and then further antibody selection and/or antibody maturation (i.e. optimization) are accomplished with the aid of one or more in-vitro techniques. According to one embodiment, a combined method of this kind may comprise firstly immunizing a nonhuman animal (e.g. a mouse, rat, rabbit, chicken, camelid, sheep or goat or a transgenic version thereof or a chimeric mouse) with said oligomer or derivative to stimulate an antibody response to the antigen and then preparing and screening a phage display antibody library by using immunoglobulin sequences of lymphocytes which have been stimulated in vivo by the action of said oligomer or derivative. The first step of this combined procedure may be carried out in the manner described above in connection with the in-vivo approaches, while the second step of this procedure may be carried out in the manner described above in connection with the in-vitro approaches. Preferred methods of hyperimmunizing nonhuman animals with subsequent in-vitro screening of phage display libraries prepared from said stimulated lymphocytes include those described by BioSite Inc., see, for example, WO 98/47343, WO 91/17271, U.S. Pat. No. 5,427,908 and U.S. Pat. No. 5,580,717.

According to another embodiment, a combined method comprises firstly immunizing a nonhuman animal (e.g. a mouse, rat, rabbit, chicken, camelid, sheep, goat or a knockout and/or transgenic version thereof, or a chimeric mouse) with an oligomer or derivative of the invention to stimulate an antibody response to said oligomer or derivative and selecting the lymphocytes which produce the antibodies having the desired specificity by screening hybridomas (prepared, for example, from the immunized animals). The genes for the antibodies or single domain antibodies are isolated from the selected clones (by means of standardized cloning methods such as reverse transcriptase polymerase chain reaction) and subjected to in-vitro affinity maturation in order to improve thereby the binding properties of the selected antibody or the selected antibodies. The first step of this procedure may be conducted in the manner described above in connection with the in-vivo approaches, while the second step of this procedure may be conducted in the manner described above in connection with the in-vitro approaches, in particular by using methods of in-vitro affinity maturation, such as those described in WO 97/29131 and WO 00/56772.

In a further combined method, the recombinant antibodies are generated from individual isolated lymphocytes by using a procedure which is known to the skilled worker as selected lymphocyte antibody methods (SLAM) and which is described in U.S. Pat. No. 5,627,052, WO 92/02551 and Babcock, J. S. et al. (1996) Proc. Natl. Acad. Sci. USA 93:7843-7848. In this method, a nonhuman animal (e.g. a mouse, rat, rabbit, chicken, camelid, sheep, goat, or a transgenic version thereof, or a chimeric mouse) is firstly immunized in vivo with the oligomer or derivative to stimulate an immune response to said oligomer or derivative, and then individual cells secreting antibodies of interest are selected by using an antigen-specific haemolytic plaque assay. To this end, the globulomer or derivative thereof or structurally related molecules of interest may be coupled to sheep erythrocytes, using a linker such as biotin, thereby making it possible to identify individual cells secreting antibodies with suitable specificity by using the haemolytic plaque assay. Following the identification of cells secreting antibodies of interest, cDNAs for the variable regions of the light and heavy chains are obtained from the cells by reverse transcriptase PCR, and said variable regions may then be expressed in association with suitable immunoglobulin constant regions (e.g. human constant regions) in mammalian host cells such as COS or CHO cells. The host cells transfected with the amplified immunoglobulin sequences derived from in vivo-selected lymphocytes may then be subjected to further in-vitro analysis and in-vitro selection by spreading out the transfected cells, for example, in order to isolate cells expressing antibodies with the binding affinity. The amplified immunoglobulin sequences may furthermore be manipulated in vitro.

Antibodies having the required affinities defined herein can be selected by performing a dot blot essentially as described above. Briefly, the antigen is attached to a solid matrix, preferably dotted onto a nitrocellulose membrane, in serial dilutions. The immobilized antigen is then contacted with the antibody of interest followed by detection of the latter by means of an enzyme-conjugated secondary antibody and a colorimetric reaction; at defined antibody and antigen concentrations, the amount of antibody bound allows affinity determination. Thus the relative affinity of two different antibodies to one target, or of one antibody to two different targets, is here defined as the relation of the respective amounts of target-bound antibody observed with the two antibody-target combinations under otherwise identical dot blot conditions.

Antibody moieties such as Fab and F(ab')₂ fragments may be produced from whole antibodies by using conventional techniques such as digestion with papain or pepsin. In addition, antibodies, antibody moieties and immunoadhesion molecules may be obtained by using standardized recombinant DNA techniques.

In a further aspect, the invention also relates to the use of the Aβ(X-38 . . . 43) oligomer or derivative of the invention for providing an aptamer that binds to the oligomer or derivative (hereinafter also referred to as anti-oligomer aptamer). Accordingly, the invention relates also to a method for providing an aptamer having specificity for the Aβ(X-38 . . . 43) oligomer or derivative as defined herein, which method comprises at least the steps of

a) providing a binding target comprising the oligomer or derivative;
b) exposing an aptamer repertoire or potential aptamer repertoire to said binding target; and
c) selecting from said repertoire an aptamer which specifically binds to said oligomer or derivative.

An “aptamer” herein refers to oligonucleic acid or peptide molecules that are capable of specific, non-covalent binding to its target. Apatmer preferably comprise peptide, DNA or

RNA sequence, more preferably peptide, DNA or RNA sequence of about 3 to 100 monomers, which may at one end or both ends be attached to a larger molecule, preferably a larger molecule mediating biochemical functions, more preferably a larger molecule inducing inactivation and/or degradation, most preferably ubiquitin, or preferably a larger molecule facilitating destruction, more preferably an enzyme or a fluorescent protein.

Here it is to be understood that a “potential aptamer repertoire” refers to any library, collection, assembly or set of amino acid sequences or nucleic acid sequences or to any generator of such a library, collection, assembly or set of amino acid sequences that can be used for producing an aptamer repertoire in vivo or in vitro.

In another aspect, the invention also provides aptamers that bind to the Aβ(X-38 . . . 43) oligomer or derivative as defined herein.

In a preferred embodiment of the invention, the aptamer is obtainable by a method comprising selecting the aptamer from a repertoire or potential repertoire as described herein.

According to a particularly preferred embodiment, the present invention provides oligomer-specific aptamers. These include in particular aptamers having a comparatively smaller affinity for both the monomeric and fibrillomeric forms of Aβ peptide than for the Aβ(X-38 . . . 43) oligomer or derivative of the invention.

The agents that are capable of binding to the Aβ(X-38 . . . 43) oligomer or derivative of the invention also have many potential applications, some of which are described in the following. They are especially useful for therapeutic and diagnostic purposes.

Antibodies that specifically bind to the globulomer epitope have proven to be useful agents in therapeutic and diagnostic applications. As the Aβ(X-38 . . . 43) oligomers or derivatives of the present invention react with said antibodies the oligomers are believed to display the same or a very similar epitope.

Thus, the invention also provides agents that are capable of binding to the Aβ(X-38 . . . 43) oligomer or derivative of the invention for therapeutic uses.

In one aspect, the invention also provides therapeutic compositions comprising an agent that is capable of binding to the Aβ(X-38 . . . 43) oligomer or derivative of the invention. According to a particular embodiment, said compositions are pharmaceutical compositions which further comprise a pharmaceutical acceptable carrier.

Said pharmaceutical compositions of the invention may furthermore contain at least one additional therapeutic agent, for example one or more additional therapeutic agents for the treatment of a disease for whose relief the agents of the invention are useful. If, for example, the agent of the invention binds to an oligomer or derivative of the invention, the pharmaceutical composition may furthermore contain one or more additional therapeutic agents useful for the treatment of disorders in which the activity of said oligomer or derivative is important.

Pharmaceutically suitable carriers include any solvents, dispersing media, coatings, antibacterial and antifungal agents, isotonic and absorption-delaying agents, and the like, as long as they are physiologically compatible. Pharmaceutically acceptable carriers include, for example, water, saline, phosphate-buffered saline, dextrose, glycerol, ethanol and the like, and combinations thereof. In many cases, preference is given to using isotonic agents, for example sugars, polyalcohols such as mannitol or sorbitol, or sodium chloride in addition. Pharmaceutically suitable carriers may furthermore contain relatively small amounts of auxiliary substances such as wetting agents or emulsifiers, preservatives or buffers, which increase the half life or efficacy of the antibodies.

The pharmaceutical compositions may be suitable for parenteral administration, for example. Here, the agents are prepared preferably as injectable solutions with an agent, e.g. antibody, content of 0.1-250 mg/ml. The injectable solutions may be prepared in liquid or lyophilized form, the dosage form being a flint glass or vial, an ampoule or a filled syringe. The buffer may contain L-histidine (1-50 mM, preferably 5-10 mM) and have a pH of 5.0-7.0, preferably of 6.0. Further suitable buffers include, without being limited thereto, sodium succinate, sodium citrate, sodium phosphate or potassium phosphate buffers. Sodium chloride may be used in order to adjust the tonicity of the solution to a concentration of 0-300 mM (preferably 150 mM for a liquid dosage form). Cryoprotectants, for example sucrose (e.g. 0-10%, preferably 0.5-1.0%) may also be included for a lyophilized dosage form. Other suitable cryoprotectants are trehalose and lactose. Fillers, for example mannitol (e.g. 1-10%, preferably 2-4%) may also be included for a lyophilized dosage form. Stabilizers, for example L-methionine (e.g. 51-50 mM, preferably 5-10 mM) may be used both in liquid and lyophilized dosage forms. Further suitable fillers are glycine and arginine. Surfactants, for example polysorbate 80 (e.g. 0-0.05%, preferably 0.005-0.01%), may also be used. Further surfactants are polysorbate 20 and BRIJ surfactants.

The compositions of the invention may have a multiplicity of forms. These include liquid, semisolid and solid dosage forms, such as liquid solutions (e.g. injectable and infusible solutions), dispersions or suspensions, tablets, pills, powders, liposomes and suppositories. The preferred form depends on the intended type of administration and on the therapeutic application. Typically, preference is given to compositions in the form of injectable or infusible solutions, for example compositions which are similar to antibodies for passive immunization of humans. The preferred route of administration is parenteral (e.g. intravenous, subcutaneous, intraperitoneal or intramuscular). According to a preferred embodiment, the agent is administered by intravenous infusion or injection. According to another preferred embodiment, the agent is administered by intramuscular or subcutaneous injection.

Therapeutic compositions must typically be sterile and stable under preparation and storage conditions. The compositions may be formulated as solutions, microemulsions, dispersions, liposomes or other ordered structures suitable for high concentrations of active substance. Sterile injectable solutions may be prepared by introducing the active compound (i.e. the agent such as an antibody) in the required amount into a suitable solvent, where appropriate with one or a combination of the abovementioned ingredients, as required, and then sterile-filtering said solution. Dispersions are usually prepared by introducing the active compound into a sterile vehicle containing a basic dispersion medium and, where appropriate, other required ingredients. In the case of a sterile lyophilized powder for preparing sterile injectable solutions, vacuum drying and spray drying are preferred methods of preparation, which produces a powder of the active ingredient and, where appropriate, of further desired ingredients from a previously sterile-filtered solution. The correct flowability of a solution may be maintained by using, for example, a coating such as lecithin, by maintaining, in the case of dispersions the required particle size or by using surfactants. A prolonged absorption of injectable compositions may be achieved by additionally introducing into the composition an agent which delays absorption, for example monostearate salts and gelatine.

The agents of the invention may be administered by a multiplicity of methods known to the skilled worker, although the preferred type of administration for many therapeutic applications is subcutaneous injection, intravenous injection or infusion. The skilled worker will appreciate that the route and/or type of administration depend on the result desired. According to particular embodiments, the active compound may be prepared with a carrier which protects the compound against rapid release, such as, for example, a formulation with sustained or controlled release, which includes implants, transdermal plasters and microencapsulated release systems. Biologically degradable biocompatible polymers such as ethylene vinyl acetate, polyanhydrides, polyglycolic acid, collagen, polyorthoesters and polylactic acid may be used. The methods of preparing such formulations are well known to the skilled worker; see, for example, Sustained and Controlled Release Drug Delivery Systems, J. R. Robinson, ed., Marcel Dekker, Inc., New York, 1978.

According to particular embodiments, an agent of the invention may be administered orally, for example in an inert diluent or a metabolizable edible carrier. The agent (and further ingredients, if desired) may also be enclosed in a hard or soft gelatine capsule, compressed to tablets or added directly to food. For oral therapeutic administration, the agents may be mixed with excipients and used in the form of oral tablets, buccal tablets, capsules, elixirs, suspensions, syrups and the like. If it is intended to administer an agent of the invention via a route other than the parenteral one, it may be necessary to choose a coating from a material which prevents its inactivation.

In a further aspect, the invention provides the use of an agent that is capable of binding to the Aβ(X-38 . . . 43) oligomer or derivative of the invention for preparing a pharmaceutical composition for treating or preventing an amyloidosis.

In a preferred embodiment of the invention, the pharmaceutical composition is for passive immunization.

Accordingly, the invention also provides a method of treating or preventing an amyloidosis in a subject in need thereof, which comprises administering the agent that is capable of binding to the Aβ(X-38 . . . 43) oligomer or derivative of the invention to the subject.

In a preferred embodiment of the invention, administering the agent that is capable of binding to the Aβ(X-38 . . . 43) oligomer or derivative of the invention is for passively immunizing the subject against an amyloidosis.

The screening of biological samples revealed that such samples may contain substances that react with agents that are capable of binding to the Aβ(X-38 . . . 43) oligomer or derivative such as anti-oligomer antibodies of the invention. Such substances which have a certain binding affinity to said agents but which cannot be said to correspond to the Aβ(X-38 . . . 43) oligomers or derivatives of the invention, are hereinafter referred to as antigens comprising the Aβ(X-38 . . . 43) oligomer epitope.

Thus, the agents that are capable of binding to the Aβ(X-38 . . . 43) oligomer or derivative of the invention are also capable of detecting, both in vitro and in vivo, antigens comprising Aβ(X-38 . . . 43) oligomer epitopes to which they bind. Said agents may therefore be used for detecting said antigens, for instance a sample that is derived from a subject suspect of having an amyloidosis, or in a subject suspect of having an amyloidosis, for instance a human individual or other mammal.

The invention thus also provides agents that are capable of binding to the Aβ(X-38 . . . 43) oligomer or derivative of the invention for diagnostic uses.

In one aspect, the invention provides diagnostic compositions comprising an agent that is capable of binding to the Aβ(X-38 . . . 43) oligomer or derivative of the invention. According to a particular embodiment, said compositions are pharmaceutical compositions which further comprise a pharmaceutical acceptable carrier.

In a further aspect, the invention provides the use of an agent that is capable of binding to the Aβ(X-38 . . . 43) oligomer or derivative of the invention for preparing a composition for diagnosing an amyloidosis.

Accordingly, the invention also provides a method of diagnosing an amyloidosis which comprises providing a sample from the subject suspected of having the amyloidosis, contacting the sample with an agent that is capable of binding to the Aβ(X-38 . . . 43) oligomer or derivative of the invention for a time and under conditions sufficient for the formation of a complex comprising the agent that is capable of binding to the Aβ(X-38 . . . 43) oligomer or derivative of the invention and an antigen comprising the Aβ(X-38 . . . 43) oligomer epitope, the presence of the complex indicating the subject has the amyloidosis. According to a particular embodiment, at least the step of contacting the sample is carried out ex vivo and in particular in vitro.

Thus, the agents that are capable of binding to the Aβ(X-38 . . . 43) oligomer or derivative of the invention may be used in a variety of diagnostic methods and assays.

According to one embodiment, the method of diagnosing an amyloidosis in a patient suspected of having this disease comprises the steps of: 1) isolating a biological sample from the patient; 2) contacting the biological sample with at least one of the agents that are capable of binding to the Aβ(X-38 . . . 43) oligomer or derivative of the invention for a time and under conditions sufficient for formation of antigen/agent complexes; and 3) detecting presence of the antigen/agent complexes in said sample, presence of the complexes indicating a diagnosis of an amyloidosis, e.g. Alzheimer's disease, in the patient. The antigen is one comprising the Aβ(X-38 . . . 43) oligomer epitope. According to a particular embodiment, at least one of said steps 2) and 3) is carried out ex vivo and in particular in vitro. According to a further particular embodiment, the method does not comprise step 1).

According to a further embodiment, the method of diagnosing an amyloidosis in a patient suspected of having this disease comprises the steps of: 1) isolating a biological sample from the patient; 2) contacting the biological sample with an antigen for a time and under conditions sufficient for the formation of antibody/antigen complexes; 3) adding a conjugate to the resulting antibody/antigen complexes for a time and under conditions sufficient to allow the conjugate to bind to the bound antibody, wherein the conjugate comprises one of the agents that are capable of binding to the Aβ(X-38 . . . 43) oligomer or derivative of the invention, attached to a signal generating compound capable of generating a detectable signal; and 4) detecting the presence of an antibody which may be present in the biological sample, by detecting a signal generated by the signal generating compound, the signal indicating a diagnosis of an amyloidosis, e.g. Alzheimer's disease in the patient. The antigen is one comprising the Aβ(X-38 . . . 43) oligomer epitope. According to a particular embodiment, at least one of said steps 2), 3) and 4) is carried out ex vivo and in particular in vitro. According to a further particular embodiment, the method does not comprise step 1).

According to a further embodiment, the method of diagnosing an amyloidosis in a patient suspected of having this disease comprises the steps of: 1) isolating a biological sample from said patient; 2) contacting the biological sample with anti-antibody, wherein the anti-antibody is specific for one of the agents that are capable of binding to the Aβ(X-38 . . . 43) oligomer or derivative of the invention, for a time and under conditions sufficient to allow for formation of anti-antibody/agent complexes, the complexes containing agent present in the biological sample; 3) adding a conjugate to resulting anti-antibody/agent complexes for a time and under conditions sufficient to allow the conjugate to bind to bound agent, wherein the conjugate comprises an antigen comprising the Aβ(X-38 . . . 43) oligomer epitope, which binds to a signal generating compound capable of generating a detectable signal; and 4) detecting a signal generated by the signal generating compound, the signal indicating a diagnosis of an amyloidosis, e.g. Alzheimer's disease in the patient. According to a particular embodiment, at least one of said steps 2), 3) and 4) is carried out ex vivo and in particular in vitro. According to a further particular embodiment, the method does not comprise step 1).

In one diagnostic embodiment of the present invention, the agent that is capable of binding to the Aβ(X-38 . . . 43) oligomer or derivative of the invention, or a portion thereof, is coated on a solid phase (or is present in a liquid phase). The test or biological sample (e.g., whole blood, cerebrospinal fluid, serum, etc.) is then contacted with the solid phase. If antigen (e.g., globulomer) is present in the sample, such antigens bind to the agents that are capable of binding to the Aβ(X-38 . . . 43) oligomer or derivative of the invention on the solid phase and are then detected by either a direct or indirect method. The direct method comprises simply detecting presence of the complex itself and thus presence of the antigens. In the indirect method, a conjugate is added to the bound agent. The conjugate comprises a second antibody, which binds to the bound antigen, attached to a signal-generating compound or label. Should the second antibody bind to the bound antigen, the signal-generating compound generates a measurable signal. Such signal then indicates presence of the antigen in the test sample.

Examples of solid phases used in diagnostic immunoassays are porous and non-porous materials, latex particles, magnetic particles, microparticles (see U.S. Pat. No. 5,705,330), beads, membranes, microtiter wells and plastic tubes. The choice of solid phase material and method of labeling the antigen or antibody present in the conjugate, if desired, are determined based upon desired assay format performance characteristics.

As noted above, the conjugate (or indicator reagent) will comprise an antibody (or perhaps anti-antibody, depending upon the assay), attached to a signal-generating compound or label. This signal-generating compound or “label” is itself detectable or may be reacted with one or more additional compounds to generate a detectable product. Examples of signal-generating compounds include chromogens, radioisotopes (e.g., 125I, 131I, 32P, 3H, 35S and 14C), chemiluminescent compounds (e.g., acridinium), particles (visible or fluorescent), nucleic acids, complexing agents, or catalysts such as enzymes (e.g., alkaline phosphatase, acid phosphatase, horseradish peroxidase, beta-galactosidase and ribonuclease). In the case of enzyme use (e.g., alkaline phosphatase or horseradish peroxidase), addition of a chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

Examples of biological fluids which may be tested by the above immunoassays include plasma, whole blood, dried whole blood, serum, cerebrospinal fluid or aqueous or organo-aqueous extracts of tissues and cells.

Kits are also included within the scope of the present invention. More specifically, the present invention includes kits for determining the presence of antigens comprising the Aβ(X-38 . . . 43) oligomer epitope in a subject. In particular, a kit for determining the presence of said antigens in a sample comprises a) an agent that is capable of binding to the Aβ(X-38 . . . 43) oligomer or derivative of the invention; and optionally b) a conjugate comprising an antibody that binds to the agent, attached to a signal generating compound capable of generating a detectable signal. The kit may also contain a control or calibrator which comprises a reagent which binds to the antigen.

The present invention also includes another type of kit for detecting antibodies such as auto-antibodies in a sample. The kit may comprise a) an antibody specific for the agent that is capable of binding to the Aβ(X-38 . . . 43) oligomer or derivative of the invention (e.g. an anti-antibody), and b) an antigen comprising the Aβ(X-38 . . . 43) oligomer epitope as defined herein. A control or calibrator comprising a reagent which binds to the antigen may also be included. More specifically, the kit may comprise a) an anti-antibody specific for the auto-antibody and b) a conjugate comprising antigen comprising the Aβ(X-38 . . . 43) oligomer epitope as defined herein, the conjugate being attached to a signal generating compound capable of generating a detectable signal. Again, the kit may also comprise a control or calibrator comprising a reagent which binds to the antigen.

The kit may also comprise one container such as vial, bottles or strip, with each container with a pre-set solid phase, and other containers containing the respective conjugates. These kits may also contain vials or containers of other reagents needed for performing the assay, such as washing, processing and indicator reagents.

Due to their binding affinity to the agents that are capable of binding to the Aβ(X-38 . . . 43) oligomer or derivative of the invention, said antigens comprising Aβ(X-38 . . . 43) oligomer epitopes can be detected in preparations suspected of containing such epitopes, their amount can be determined in said preparations and they can be enriched. Accordingly, the present invention also provides a method for detecting, for determining the amount of and/or for enriching Aβ(X-38 . . . 43) oligomer epitopes in preparations suspected or know to comprise such epitopes. Once detected and enriched, said substances may have potential applications similar to those described herein with respect to the Aβ(X-38 . . . 43) oligomers or derivatives of the invention.

Moreover, the present invention includes a method of designing agents such as antibodies, non-antibody biological agents or small molecules useful in the treatment or prevention of an amyloidosis in a patient. This method comprises the steps of: a) analyzing the three-dimensional structure of the oligomer of derivative described herein; b) identifying one or more epitopes on the surface of the oligomer or derivative of step a); and c) designing an agent such as an antibody, non-antibody biological agent or a small molecule which will bind to the identified epitope or epitopes of step b), the antibody, non-antibody biological agent or a small molecule to be used in the treatment or prevention of amyloidosis.

### ADVANTAGES OF THE INVENTION

The amino acid composition of the Aβ(X-38 . . . 43) oligomers or derivatives of the present invention is well-defined and reproducible. There is no risk of contamination with proteases.

Active immunization with the Aβ(X-38 . . . 43) oligomers or derivatives of the present invention elicits a highly selective immune response for Aβ globulomers. Because the Aβ(X-38 . . . 43) oligomers or derivatives of the present invention lack N-terminal sequences, there is no risk of eliciting an unspecific N-terminal Aβ peptide directed immune response. The Aβ(X-38 . . . 43) oligomers or derivatives of the present invention are therefore capable of eliciting an immune response that discriminates other forms of Aβ peptides, particularly monomers and fibrils.

As a rule, the larger and the more hydrophobic the immunogen the better is the immune response. Thus, the high molecular weight of the Aβ(X-38 . . . 43) oligomers or derivatives of the present invention combined with the hydrophobicity of Aβ(20-42) peptide reliably elicits a robust immune response in active immunization.

Further, it is expected that active immunization with the Aβ(X-38 . . . 43) oligomers or derivatives of the present invention will be effective in reversing cognitive deficits in AD transgenic mouse models as the elicited antibody response is comparable to active immunization with Aβ(20-42) truncated globulomer. The latter has been proven to reverse deficits in novel object recognition task.

The process for preparing the Aβ(X-38 . . . 43) oligomers or derivatives of the present invention does not require a step of proteolytic cleavage.

All patents, patent applications and publications referred to herein are hereby incorporated in their entirety by reference.

Deposit Information: The hybridoma which produces monoclonal antibody 5F7 was deposited with the American Type Culture Collection, 10801 University Boulevard, Manassas, Va. 20110 on Dec. 1, 2005 under the terms of the Budapest Treaty and received designation PTA-7241. Further, the hybridoma which produces monoclonal antibody 10F11 was deposited with the American Type Culture Collection, 10801 University Boulevard, Manassas, Va. 10801 on Dec. 1, 2005 under the terms of the Budapest Treaty and received designation PTA-7239. Additionally, the hybridoma which produces monoclonal antibody 4B7 was deposited with the American Type Culture Collection, 10801 University Boulevard, Manassas, Va. 10801 on Dec. 1, 2005 under the terms of the Budapest Treaty and received designation PTA-7242, and the hybridoma which produces monoclonal antibody 7C6 was deposited with the American Type Culture Collection, 10801 University Boulevard, Manassas, Va. 10801 on Dec. 1, 2005 under the terms of the Budapest Treaty and received designation PTA-7240. Additionally, the hybridoma which produces monoclonal antibody 6A2 was deposited with the American Type Culture Collection, 10801 University Boulevard, Manassas, Va. 10801 on Feb. 28, 2006 under the terms of the Budapest Treaty and received designation PTA-7409, and the hybridoma which produces monoclonal antibody 2F2 was deposited with the American Type Culture Collection, 10801 University Boulevard, Manassas, Va. 10801 on Feb. 28, 2006 under the terms of the Budapest Treaty and received designation PTA-7408. The hybridoma which produces monoclonal antibody 4D10 was deposited with the American Type Culture Collection, 10801 University Boulevard, Manassas, Va. 10801 on Feb. 28, 2006 under the terms of the Budapest Treaty and received designation PTA-7405. The hybridoma which produces monoclonal antibody 7E5 was deposited with the American Type Culture Collection, 10801 University Boulevard, Manassas, Va. 10801 on Aug. 16, 2006 under the terms of the Budapest Treaty and received designation PTA-7809. The hybridoma which produces monoclonal antibody 10C1 was deposited with the American Type Culture Collection, 10801 University Boulevard, Manassas, Va. 10801 on Aug. 16, 2006 under the terms of the Budapest Treaty and received designation PTA-7810. The hybridoma which produces monoclonal antibody 3B10 was deposited with the American Type Culture Collection, 10801 University Boulevard, Manassas, Va. 10801 on Sep. 1, 2006 under the terms of the Budapest Treaty and received designation PTA-7851. All deposits have been made on behalf of Abbott Laboratories, 100 Abbott Park Road, Abbott Park, Ill. 60064 (US). All these monoclonal antibodies are murine monoclonal antibodies.

The following examples are intended to illustrate the invention, without limiting its scope.

ABBREVIATIONS as used throughout the present specification, claims and figures: AD, Alzheimer's disease; Aβ, amyloid β peptide; APP, amyloid precursor protein; BSA, bovine serum albumin; CTMAB, cetyltrimethylammonium bromide; CD, circular dichroism spectroscopy; DIV, days in vitro; DMSO, dimethyl sulfoxide; DDAP, dodecyl-N,N-dimethyl-3-amino-1-propane sulfonate; GABA, γ-amino butyric acid; HFIP, 1,1,1,3,3,3-hexafluoro-2-propanol; LTP, long-term potentiation; NaH₂PO₄, sodium phosphate; PBS, phosphate buffered saline; SDS, sodium dodecyl sulfate; SELDI-MS, surface enhanced laser desorption ionization-mass spectrometry; TBS, Tris buffered saline.

The Aβ(X-38 . . . 43) oligomers of the present invention are hereinafter referred to as Aβ(X-38 . . . 43) HMW oligomers (short for Aβ(X-38 . . . 43) high molecular weight oligomers).

### EXAMPLES

### Preparation of Aβ(X-38 . . . 43) HMW Oligomer

### Example 1

### Preparation of Aβ(20-42) HMW Oligomer Using HFIP and SDS

Aβ(20-42) peptide was obtained via peptide synthesis.

30 mg of Aβ(20-42) peptide were dissolved in 1.5 mL of 1,1,1,3,3,3-hexafluoro-2-propanol (HFIP) by shaking for 1 h at 37° C. to obtain a clear solution with a concentration of 20 mg/mL Aβ(20-42) peptide.

30 mL buffer composed of 5 mM NaH₂PO₄, 35 mM NaCl, 0.25% SDS, pH 7.4, were added to the solution and mixed by vortexing. The sample was aliquoted into 30 reaction tubes (1.5 mL size) each containing 1 mL. Sample aliquots were incubated at 37° C. under shaking for 10 min. Samples were centrifuged at room temperature for 10 min at 10,000×g. The supernatant was withdrawn and each 500 μl aliquoted into 60 parallel reaction tubes (1.5 mL).

Samples were incubated for 2 h with open tube lid at 850 rpm in an orbital shaker at 37° C. The tube lids were closed and the samples were quiescently incubated for further 2 h at 37° C. Subsequently, samples were incubated 18 h at room temperature (22° C.). The samples were pooled resulting in a total volume of ±25 mL (instead of the initial 30 mL due to the evaporation of buffer and the highly volatile HFIP during the incubation step with the open lid). The pooled samples were frozen for 1 h at −30° C.

The samples were thawed, split into 2 parallel samples of each 12.5 mL and concentrated via ultracentrifugation with 30 kDa molecular weight cut off Centriprep concentrator devices (Millipore, cat. no.: 4306) to a total volume of 4 mL (2×2 mL). The concentrate was centrifuged at 10,000×g at room temperature for 10 min and the supernatant was withdrawn.

The supernatant was dialyzed against 1 L of 5 mM NaH₂PO₄, 35 mM NaCl, 0.25% SDS, pH 7.4, using dialysis tubings with 12-14 kDa molecular weight cut-off. After 2 h dialysis at room temperature the buffer was exchanged for fresh buffer and dialysis was continued for another 18 h. The dialysate was centrifuged at 10,000×g for 15 min. The supernatant was withdrawn, aliquoted and frozen at −80° C. until use.

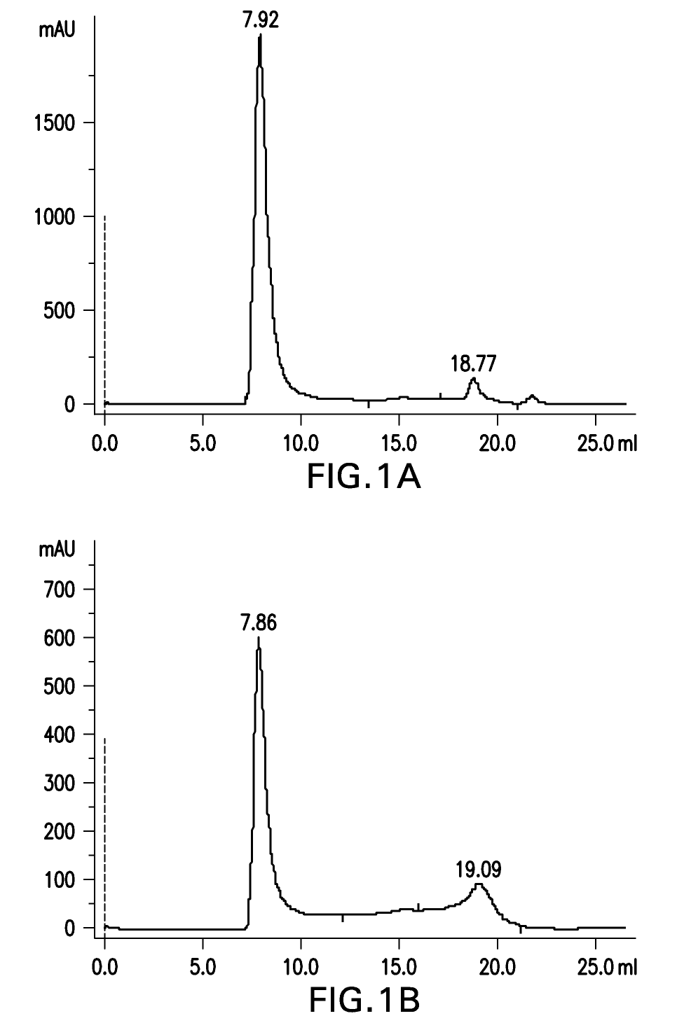

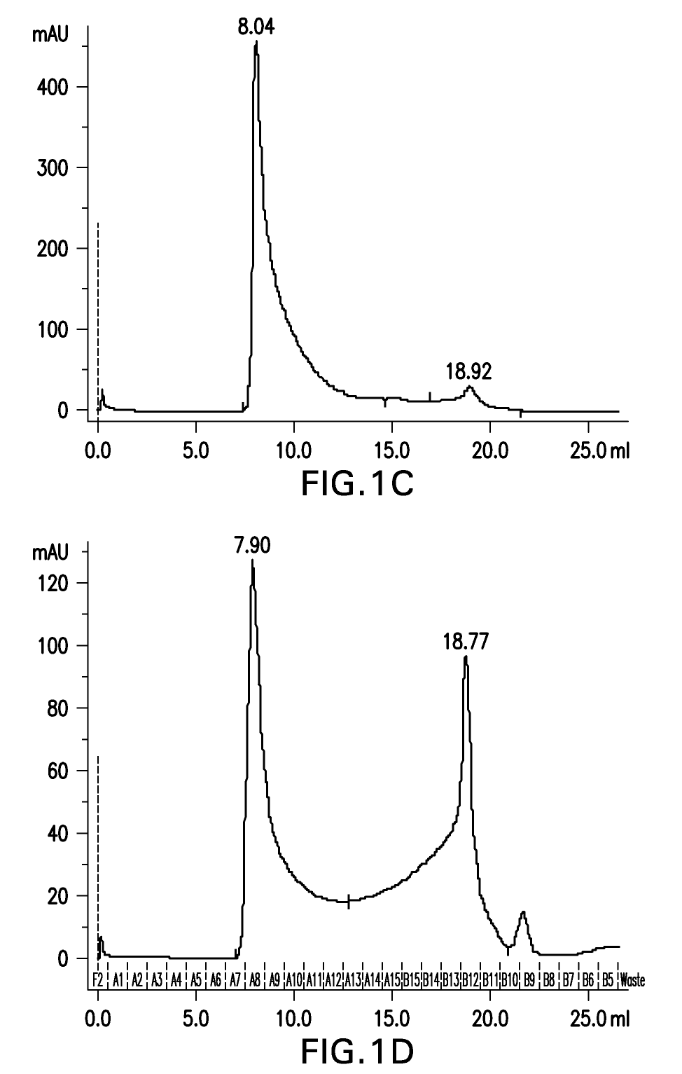

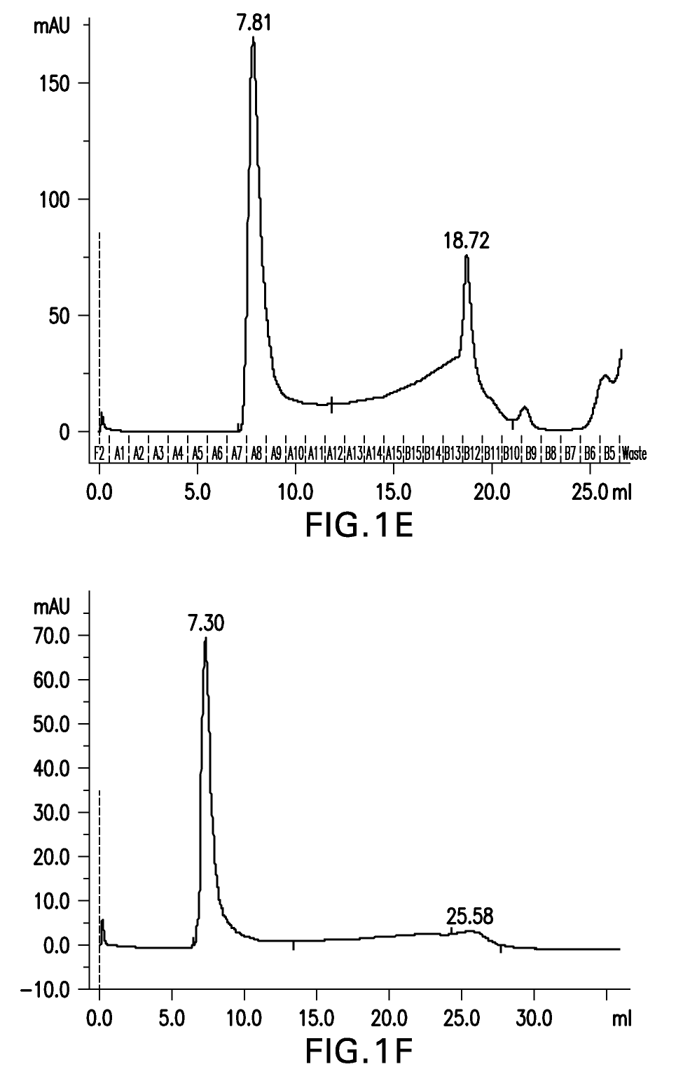

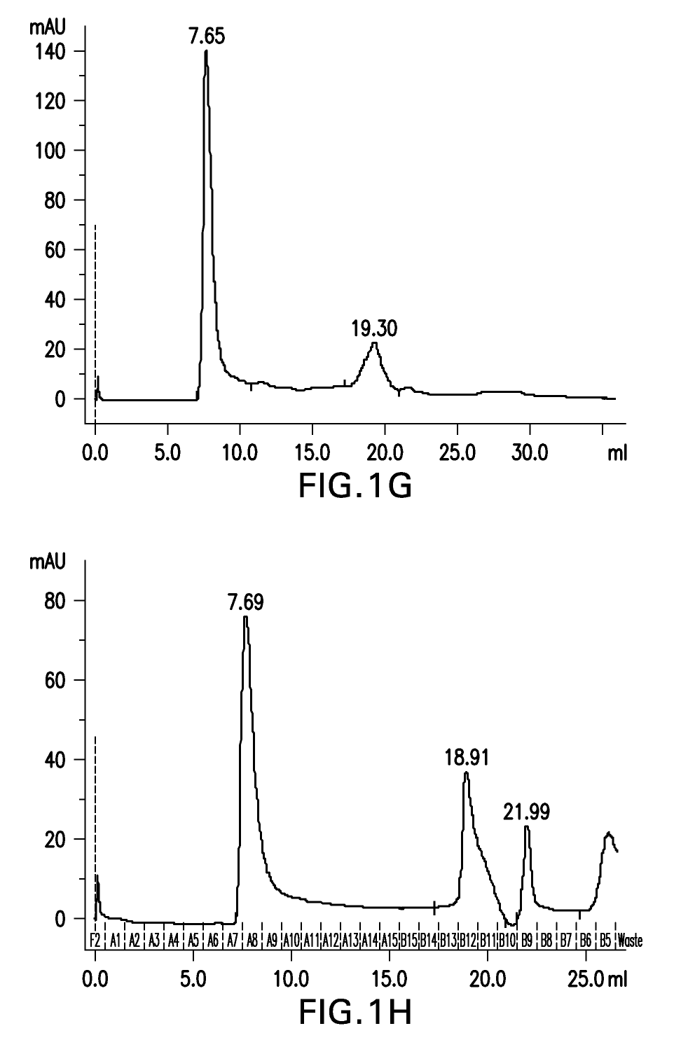

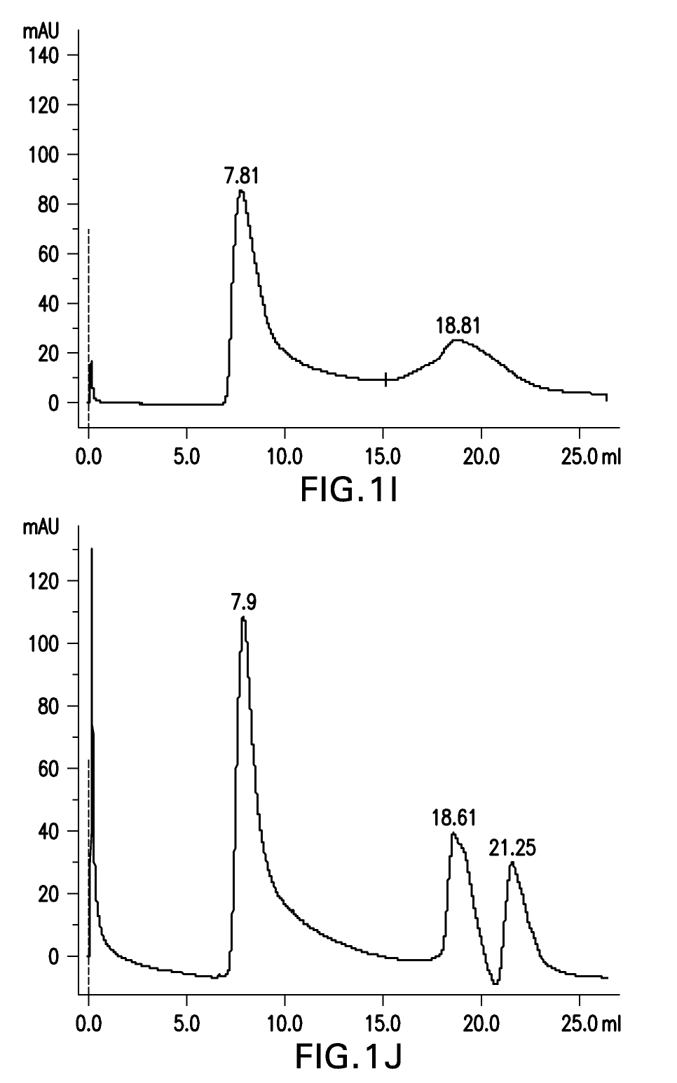

Size exclusion chromatography according to reference example 12 showed that the Aβ(20-42) HMW oligomer eluted from the column in the exclusion volume corresponding to a molecular weight of 500 kDa (FIG. 1A).

### Example 2

### Preparation of Aβ(20-42) HMW oligomer using HFIP and SDS

Aβ(20-42) HMW oligomer was prepared as in example 1, with the exception that the concentrate was desalted using a High Trap Desalting column.

Briefly, 30 mg of Aβ(20-42) peptide were dissolved in 1.5 mL of 1,1,1,3,3,3-hexafluoro-2-propanol (HFIP) by shaking for 1 h at 37° C. to obtain a clear solution with a concentration of 20 mg/mL Aβ(20-42) peptide.

30 mL buffer composed of 5 mM NaH₂PO₄, 35 mM NaCl, 0.25% SDS, pH 7.4, were added to the solution and mixed by vortexing. The sample was aliquoted into 30 reaction tubes (1.5 mL size) each containing 1 mL. Sample aliquots were incubated at 37° C. under shaking for 10 min. Samples were centrifuged at room temperature for 10 min at 10,000×g. The supernatant was withdrawn and each 500 μl aliquoted into 60 parallel reaction tubes (1.5 mL).

Samples were incubated for 2 h with open tube lid at 850 rpm in an orbital shaker at 37° C. The tube lids were closed and the samples were quiescently incubated for further 2 h at 37° C. Subsequently, samples were incubated 18 h at room temperature (22° C.). The samples were pooled resulting in a total volume of ˜25 mL (instead of the initial 30 mL due to the evaporation of buffer and the highly volatile HFIP during the incubation step with the open lid). The pooled samples were frozen for 1 h at −30° C.

The samples were thawed, split into 2 parallel samples of each 12.5 mL and concentrated via ultracentrifugation with 30 kDa molecular weight cut off Centriprep concentrator devices (Millipore, cat. no.: 4306) to a total volume of 4 mL (2×2 mL). The concentrate was centrifuged at 10,000×g at room temperature for 10 min and the supernatant was withdrawn.

The supernatant was desalted against 20 mM NaH₂PO₄, 140 mM NaCl, 0.5% SDS, pH 7.4, in 5 runs of each 500 μL sample load using a High Trap Desalting column (GE, 5 mL, cat.17-1408-01) on an Akta Purifier system (GE) with a flow rate of 1 mL/min. The first 1 mL of the UV 215 nm active peak of each run was collected, aliquoted and frozen at −80° C. until use.

Size exclusion chromatography according to reference example 12 showed that the Aβ(20-42) HMW oligomer eluted from the column in the exclusion volume corresponding to a molecular weight of ≧500 kDa (FIG. 1B).

### Example 3

### Preparation of Aβ(20-42) HMW Oligomer Using HFIP and SDS

Aβ(20-42) HMW oligomer was prepared as in example 1, with the exception that after the generation of the Aβ(20-42) HMW oligomer it was 1:4 diluted to reduce the SDS concentration from 0.25% SDS to 0.0625% SDS below the CMC in order to allow for a dialysis of SDS.

Briefly, 2 mg of Aβ(20-42) peptide were dissolved in 100 μL of 1,1,1,3,3,3-hexafluoro-2-propanol (HFIP) by shaking for 1 h at 37° C. to obtain a clear solution with a concentration of 20 mg/mL Aβ(20-42) peptide.

2 mL buffer composed of 5 mM NaH₂PO₄, 35 mM NaCl, 0.25% SDS, pH 7.4, were added to the solution and mixed by vortexing. The sample was incubated at 37° C. under shaking for 10 min. The sample was centrifuged at room temperature for 10 min at 10,000×g. The supernatant was withdrawn and each 500 μl aliquoted into 4 parallel reaction tubes (1.5 mL).

Samples were incubated for 2 h with open tube lid at 850 rpm in an orbital shaker at 37° C. The tube lids were closed and the samples were quiescently incubated for further 20 h at room temperature. Each of the 4 reaction vials contained a remaining volume of approximately 367 μl. Subsequently, 3 of the 4 reaction vials were pooled and the 4^{th} reaction tube was stored for other purposes.

The pooled sample resulted in a total volume of 1.1 ml which was diluted with 3.3 ml of 5 mM NaH₂PO₄, 35 mM NaCl, pH 7.4, and concentrated via ultracentrifugation with 30 kDa molecular weight cut off Centriprep concentrator devices (Millipore, cat. no.: 4306) to a volume of 450 μl. The concentrate was centrifuged at room temperature for 10 min at 10000×g and the supernatant was withdrawn.

The supernatant was dialyzed against 300 mL of 5 mM NaH₂PO₄, 35 mM NaCl, 0.05% SDS, pH 7.4 for 20 h at room temperature, using dialysis tubings with 12-14 kDa molecular weight cut off. The supernatant was withdrawn, aliquoted and frozen at −80° C. until use.

Size exclusion chromatography according to reference example 12 showed that the Aβ(20-42) HMW oligomer eluted from the column in the exclusion volume corresponding to a molecular weight of 500 kDa (FIG. 1C).

### Example 4

### Preparation of Aβ(20-42) HMW Oligomer Using HFIP and Lauric Acid

Aβ(20-42) HMW oligomer was prepared in analogy to example 1, with the exception that lauric acid was used instead of SDS. In this example, the last concentration/dialysis step was not carried out as it is not required to show that Aβ(20-42) HMW oligomer formation can also be achieved with lauric acid.

Briefly, 2.9 mg of Aβ(20-42) peptide were dissolved in 145 μL of 1,1,1,3,3,3-hexafluoro-2-propanol (HFIP) by shaking for 1 h at 37° C. to obtain a clear solution with a concentration of 20 mg/mL Aβ(20-42) peptide.

1 mL buffer composed of 5 mM NaH₂PO₄, 35 mM NaCl, 0.5% lauric acid, pH 8.7, were added to 50 μl of the solution and mixed by vortexing. The sample was incubated at 37° C. under shaking for 20 min. The sample was centrifuged at room temperature for 10 min at 10,000×g. The supernatant was withdrawn and each 500 μl aliquoted into 2 parallel reaction tubes (1.5 mL).

Samples were incubated for 2 h with open tube lid at 850 rpm in an orbital shaker at 37° C. The tube lids were closed and the samples were quiescently incubated for further 20 h at 37° C. The samples were pooled and centrifuged at room temperature for 10 min at 10,000×g. The supernatant was withdrawn and frozen for 30 min at −30° C. Subsequently, the sample was thawed and then centrifuged for 10 min at 10,000×g. The supernatant was withdrawn and frozen at −80° C. until use.

Size exclusion chromatography according to reference example 12 showed that the Aβ(20-42) HMW oligomer eluted from the column in the exclusion volume corresponding to a molecular weight of 500 kDa (FIG. 1D).

### Example 5

### Preparation of Aβ(20-42) HMW Oligomer Using HFIP and N-Lauroylsarcosine

Aβ(20-42) HMW oligomer was prepared in analogy to example 1, with the exception that N-lauroylsarcosine was used instead of SDS. In this example, the last concentration/dialysis step was not carried out as it is not required to show that Aβ(20-42) HMW oligomer formation can also be achieved with N-lauroylsarcosine.

Briefly, 2.9 mg of Aβ(20-42) peptide were dissolved in 145 μL of 1,1,1,3,3,3-hexafluoro-2-propanol (HFIP) by shaking for 1 h at 37° C. to obtain a clear solution with a concentration of 20 mg/mL Aβ(20-42) peptide.

1 mL buffer composed of 5 mM NaH₂PO₄, 35 mM NaCl, 0.5% N-lauroylsarcosine, pH 8.7, were added to 50 μl of the solution and mixed by vortexing. The sample was incubated at 37° C. under shaking for 20 min. The sample was centrifuged at room temperature for 10 min at 10,000×g. The supernatant was withdrawn and each 500 μl aliquoted into 2 parallel reaction tubes (1.5 mL).

Samples were incubated for 2 h with open tube lid at 850 rpm in an orbital shaker at 37° C. The tube lids were closed and the samples were quiescently incubated for further 20 h at 37° C. The samples were pooled and centrifuged at room temperature for 10 min at 10,000×g. The supernatant was withdrawn and frozen for 30 min at −30° C. Subsequently, the sample was thawed and then centrifuged for 10 min at 10,000×g. The supernatant was withdrawn and frozen at −80° C. until use.

Size exclusion chromatography according to reference example 12 showed that the Aβ(20-42) HMW oligomer eluted from the column in the exclusion volume corresponding to a molecular weight of 500 kDa (FIG. 1E).

### Example 6

### Preparation of Aβ(22-42) HMW Oligomer Using HFIP and SDS

Aβ(22-42) HMW oligomer was prepared in analogy to example 3, with the exception that Aβ(22-42) peptide was used instead of Aβ(20-42) peptide. In this example, the last dialysis step was not carried out as it is not required to show that Aβ(22-42) HMW oligomer formation can be achieved in analogy to example 3.

Briefly, 1 mg of Aβ(22-42) peptide were dissolved in 50 μL of 1,1,1,3,3,3-hexafluoro-2-propanol (HFIP) by shaking for 1.5 h at 37° C. to obtain a clear solution with a concentration of 20 mg/mL Aβ(22-42) peptide.

1 mL buffer composed of 5 mM NaH₂PO₄, 35 mM NaCl, 0.2% SDS, pH 7.4, were added to the solution and mixed by vortexing. The sample was incubated at 37° C. under shaking for 10 min. The sample was centrifuged at room temperature for 10 min at 10,000×g. The supernatant was withdrawn and each 500 μl aliquoted into 2 parallel reaction tubes (1.5 mL).

Samples were incubated for 2 h with open tube lid at 850 rpm in an orbital shaker at 37° C. The tube lids were closed and the samples were quiescently incubated for further 20 h at 37° C. The 2 reaction vials contained a remaining volume of approximately each 400 μl. Subsequently, 1 of the 2 reaction vials was stored for other purposes.

The sample in the second reaction vial was diluted with 1.2 ml of 5 mM NaH₂PO₄, 35 mM NaCl, pH 7.4, and concentrated via ultracentrifugation with 30 kDa molecular weight cut off Ultra Free MC filter devices (Millipore, cat. no.: UFC3LTK00) to a volume of 75 μl. The concentrate was frozen at −80° C.

Size exclusion chromatography according to reference example 12 showed that the Aβ(20-42) HMW oligomer eluted from the column in the exclusion volume corresponding to a molecular weight of 500 kDa (FIG. 1F).

### Example 7

### Preparation of Aβ(20-42) HMW Oligomer Using NaOH and SDS

Aβ(20-42) HMW oligomer was prepared in analogy to example 1, with the exception that Aβ(20-42) peptide was dissolved in 0.1% NaOH instead of HFIP. In this example, the last concentration/dialysis step was not carried out as it is not required to show that Aβ(20-42) HMW oligomer formation can be achieved in analogy to example 1.

Briefly, 1.2 mg of Aβ(20-42) peptide were dissolved in 120 μL of 0.1% NaOH by shaking for 1 min at room temperature to obtain a clear solution with a concentration of 10 mg/mL Aβ(20-42) peptide.

90 μL buffer composed of 5 mM NaH₂PO₄, 35 mM NaCl, 0.5% SDS, pH 7.4, were added to 10 μl of the solution and mixed by vortexing. The pH was adjusted to pH 7.4 with 2% HCl in water. The sample was incubated at room temperature under shaking for 5 min and then centrifuged at room temperature for 10 min at 10,000×g. The supernatant was withdrawn and incubated at room temperature for 20 h.

Size exclusion chromatography according to reference example 12 showed that the Aβ(20-42) HMW oligomer eluted from the column in the exclusion volume corresponding to a molecular weight of 500 kDa (FIG. 1G).

### Example 8

### Preparation of Aβ(20-42) HMW Oligomer Using NaOH and N-Lauroylsarcosine as Detergent

Aβ(20-42) HMW oligomer was prepared in analogy to example 7, with the exception that N-lauroylsarcosine was used instead of SDS. In this example, the last concentration/dialysis step was not carried out as it is not required to show that Aβ(20-42) HMW oligomer formation can be achieved in analogy to example 1.

Briefly, 1.2 mg of Aβ(20-42) peptide were dissolved in 120 μL of 0.1% NaOH by shaking for 1 min at room temperature to obtain a clear solution with a concentration of 10 mg/mL Aβ(20-42) peptide.

225 μL buffer composed of 5 mM NaH₂PO₄, 35 mM NaCl, 0.5% N-lauroylsarcosine, pH 7.4, were added to 25 μl of the solution and mixed by vortexing. The pH was adjusted to pH 7.4 with 2% HCl in water. The sample was incubated at room temperature under shaking for 5 min and then centrifuged at room temperature for 10 min at 10,000×g. The supernatant was withdrawn and incubated at room temperature for 20 h.

Size exclusion chromatography according to reference example 12 showed that the Aβ(20-42) HMW oligomer eluted from the column in the exclusion volume corresponding to a molecular weight of 500 kDa (FIG. 1H).

### Example 9

### Preparation of Aβ(20-42) HMW Oligomer Using NaOH and 0.2% Tween 80 Aβ(20-42) Peptide was Obtained via Peptide Synthesis

30 mg of Aβ(20-42) peptide were dissolved in 2.4 mL of 0.1% NaOH by shaking for 5 min at room temperature to obtain a solution with a concentration of 12.5 mg/mL Aβ(20-42) peptide. The sample was centrifuged for 5 min at 10,000 g. Then the supernatant was withdrawn and 5.1 mL of 5 mM NaH₂PO₄, 35 mM NaCl, 0.2% Tween 80, pH7.4 were added to the supernatant. The pH was adjusted with 1% HCl to pH 7.60. The sample was incubated under stirring for 2 h at 37° C. The sample was aliquoted and frozen at −80° C. until use.

Size exclusion chromatography according to reference example 12 (20 μl of sample were diluted with 180 μl of mobile phase and 10 μl of this mixture were loaded) showed that the Aβ(20-42) HMW oligomer eluted from the column in the exclusion volume corresponding to a molecular weight of 500 kDa (FIG. 11).

### Example 10

### Preparation of Aβ(20-42) HMW Oligomer in Buffer at pH 8.5 Aβ(20-42) Peptide was Obtained via Peptide Synthesis

3 mg of Aβ(20-42) peptide were dissolved in 0.24 mL of 0.1% NaOH by shaking for 1 min at room temperature to obtain a solution with a concentration of 12.5 mg/mL of Aβ(20-42) peptide. The sample was centrifuged for 5 min at 10,000 g. The supernatant was withdrawn and 0.51 mL of 5 mM NaH₂PO₄, 35 mM NaCl, pH 7.4 were added to the supernatant. The pH was adjusted with 1% HCl from pH 8.8 to pH 8.5. The sample was incubated for 20 h at 37° C. The pH was then adjusted with 1% HCl to pH 7.4. The sample was aliquoted and frozen at ±80° C. until use.

Size exclusion chromatography according to reference example 12 (50 μl of sample were diluted with 50 μl of mobile phase and 50 μl of this mixture were loaded) showed that the Aβ(20-42) HMW oligomer eluted from the column in the exclusion volume corresponding to a molecular weight of 500 kDa (FIG. 1J).

### Example 12

### Aβ(20-42) HMW Oligomer Formation Using Different Amphipathic Agents

The capability of various amphipathic inducing agents to induce Aβ(20-42) HMW oligomer formation was tested. To this end, Aβ(20-42) peptide was incubated with the different amphipathic agents. The amphipathic agents were used at a concentration above the critical micellar concentration. Aβ(20-42) HMW oligomer formation was analyzed with size exclusion chromatography (SEC) and the relative amount of formed Aβ(20-42) HMW oligomer calculated from the 500 kDa peak area of the SEC-chromatogram.

### Preparation of Aβ(20-42) HMW Oligomers with Initial NaOH Solubilization

In this experiment the Aβ(20-42) peptide was initially solubilized in 0.1% NaOH and subsequently added to a solution containing the amphipatic agent.

Preparation with pH Adjusted to pH 7.4:

Aβ(20-42) peptide was obtained via peptide synthesis. 1.4 mg of the Aβ(20-42) peptide were dissolved in 140 μl 0.1% NaOH by shaking for 1 min at room temperature to obtain a clear solution with a concentration of 10 mg/mL Aβ(20-42) peptide.

To 10 μl of this solution 90 μl of buffer containing the respective concentration of the amphipathic reagent (see list below A-I) dissolved in 5 mM NaH₂PO₄, 35 mM NaCl, pH 7.4 were added and mixed by vortexing. The pH of the samples was adjusted with 2% HCl to pH 7.4 and then the samples were incubated at 37° C. under shaking for 2 h. Samples were centrifuged at room temperature for 10 min at 10,000×g. The supernatant was withdrawn and used for SEC analysis.

### List of Amphipathic Reagents:

A) 0.5% SDS
B) 0.2% Triton X 100
C) 0.2% Tergitol NP-40
D) 0.5% Lauric acid
E) 0.5% N-Lauroylsarcosine
F) 0.5% CHAPS
G) 0.25% N-Dodecyl-N,N-dimethyl-3-amin-1-propansulfonat
H) 0.5% Dodecylamin
I) 0.2% Cetyltrimethylammoniumbromid (CTMAB)
   Preparation without pH Adjustment:
J) Lauric acid (pH8.7) induced Aβ(20-42) HMW oligomer formation:

Aβ(20-42) peptide was obtained via peptide synthesis. 1.4 mg of the Aβ(20-42) peptide were dissolved in 140 μl 0.1% NaOH by shaking for 1 min at room temperature to obtain a clear solution with a concentration of 10 mg/mL Aβ(20-42) peptide.

To 10 μl of this solution 90 μl of buffer composed of 0.5% lauric acid dissolved in 5 mM NaH₂PO₄, 35 mM NaCl, pH 7.4 were added and mixed by vortexing. The measured pH of the sample was pH 8.7. The sample was incubated at 37° C. under shaking for 2 h. Sample was centrifuged at room temperature for 10 min at 10,000×g. The supernatant was withdrawn and used for SEC analysis.
K) Dodecylamin (pH 10.0) induced Aβ(20-42) HMW oligomer formation:

Aβ(20-42) peptide was obtained via peptide synthesis. 1.4 mg of the Aβ(20-42) peptide were dissolved in 140 μl 0.1% NaOH by shaking for 1 min at room temperature to obtain a clear solution with a concentration of 10 mg/mL Aβ(20-42) peptide.

To 10 μl of this solution 90 μl of buffer composed of 0.5% dodecylamin dissolved in 5 mM NaH₂PO₄, 35 mM NaCl, pH 7.4 were added and mixed by vortexing. The measured pH of the sample was pH 10.0. The sample was incubated at 37° C. under shaking for 2 h. The sample was centrifuged at room temperature for 10 min at 10,000×g. The supernatant was withdrawn and used for SEC analysis.

### SEC-Analysis:

Size exclusion chromatography was performed as described in reference example 12.

Directly prior to the SEC, 75 μl of the respective sample-supernatants were mixed with 75 μl of the SEC mobile phase (20 mM NaH₂PO₄, 140 mM NaCl, 1% SDS, pH 7.4) and 100 μl of the mixture were immediately analyzed by SEC. The peak area of the ≧500 kDa peak (at ˜8 mL elution volume) was determined using the GE analysis software (Unicorn 5.11) for the Äkta Purifier HPLC system performing a zero-baseline correction.

The results are shown in FIG. 12.

The amphipathic agents:
0.5% SDS, (pH 7.4), anionic amphipathic agent
0.2% Triton X100, (pH 7.4), neutral amphipathic agent
0.2% Tergitol NP-40, (pH 7.4), neutral amphipatic agent
0.5% N-Lauroylsarcosine (pH 7.4), anionic amphipathic agent
0.5% CHAPS (pH 7.4), hybrid ionic amphipatic agent
0.25% Dodecyl-N,N-dimethyl-3-amino-1-propansulfonat (DDAP) (pH 7.4), hybrid ionic amphipatic agent
0.5% Lauric Acid (pH 8.7), anionic amphipathic agent
   induced Aβ(20-42) HMW oligomers with a high efficiency, as analyzed by the SEC 500 kDa peak area quantification (FIG. 12A).

The amphipathic agent:

0.5% Dodecylamin (pH 10.0), cationic amphipathic agent induced Aβ(20-42) HMW oligomers with a low efficiency, as analyzed by the SEC≧500 kDa peak area quantification (FIG. 12A).

The amphipathic agents:
0.5% Lauric Acid (pH 7.4), anionic amphipathic agent
0.5% Dodecylamin (pH 7.4), cationic amphipathic agent
0.2% Cetyltrimethylammoniumbromid (pH 7.4), cationic amphipathic agent
   did not induce substantial amounts of Aβ(20-42) HMW oligomer, presumably due to the fact that these amphipathic agents itself are not soluble at the neutral pH of 7.4. If no amphipathic agent is added (in the case of the ¼ PBS (pH7.4) sample) no substantial amount of Aβ(20-42) HMW oligomer can be detected by SEC either (FIG. 12A).

### Example 13

### Kinetics of Aβ(20-42) HMW Oligomer Formation

The method for producing Aβ(20-42) HMW oligomer was carried out stepwise and Aβ(20-42) HMW oligomer formation was analyzed in several intermediate preparations by SEC.

Material that eluted with relevant size exclusion chromatography peaks was then subjected to SDS-PAGE analysis to assess Aβ(20-42) peptide content (FIG. 3).

SEC-Analysis was performed as described in reference example 12.

Briefly, 2.63 mg of Aβ(20-42) peptide were dissolved in 131 μl of 1,1,1,3,3,3-hexafluoro-2-propanol (HFIP) by shaking for 1 h at 37° C. to obtain a clear solution with a concentration of 20 mg/mL Aβ(20-42) peptide (hereinafter referred to as Aβ(20-42) HFIP solution (20 mg/mL)).

### a) Aβ(20-42) Monomer Control:

3.75 μl of the Aβ(20-42) HFIP solution (20 mg/mL) were mixed with 146 μl of the SEC mobile phase (20 mM NaH₂PO₄, 140 mM NaCl, 1% SDS, pH 7.4). 100 μl of the resulting sample were analyzed immediately by SEC.

The resulting size exclusion chromatogram (FIG. 2A) showed peaks at ˜19 ml and ˜21 ml elution volume, but no detectable peak at ˜8 ml elution volume. SDS-PAGE analysis (FIG. 3, lanes 4 and 5) revealed that the peak at ˜19 ml contained most of the peptide. The elution volume of this ˜19 ml peak equals ˜3 kDa and thus indicates monomeric Aβ(20-42) peptide. The peak at ˜21 ml contained only a minor protein fraction and likely was the salt peak plus residual HFIP.

b) Aβ(20-42) Monomer Control in Buffer Without Inducing Agent (e.g. SDS):

250 μL buffer composed of 5 mM NaH₂PO₄, 35 mM NaCl, pH 7.4 were added to 12.5 μl of the Aβ(20-42) HFIP solution (20 mg/mL) and mixed by vortexing. The sample was centrifuged at room temperature for 10 min at 10,000×g. 75 μl of the supernatant were mixed with 75 μl of the SEC mobile phase (20 mM NaH₂PO₄, 140 mM NaCl, 1% SDS, pH 7.4) and 100 μl of the mixture immediately analyzed by SEC.

The resulting size exclusion chromatogram (FIG. 2B) showed no detectable peak at ˜8 ml elution volume. SDS-PAGE analysis (FIG. 3, lanes 6 and 7) revealed that the peak at ˜23 ml contained no detectable peptide.

This indicates that the Aβ(20-42) peptide itself did not spontaneously form any high molecular weight oligomers. The loss of all Aβ(20-42) peptide after SEC analysis can be explained by the fact that upon dilution of the HFIP solubilized Aβ(20-42) peptide into the buffer composed of 5 mM NaH₂PO₄, 35 mM NaCl, pH 7.4, without any high molecular weight oligomer inducing amphipathic agent (e.g. SDS) the Aβ(20-42) peptide precipitated and was removed by subsequent centrifugation. Thus, no soluble Aβ(20-42) peptide remained in the centrifugation supernatant which represents the soluble Aβ(20-42) peptide fraction.

c) Aβ(20-42) Peptide Sample in Buffer with 0.25% SDS for 10 Min 37° C.:

250 μL buffer composed of 5 mM NaH₂PO₄, 35 mM NaCl, 0.25% SDS, pH 7.4 were added to 12.5 μl of the Aβ(20-42) HFIP solution (20 mg/mL) and the sample was incubated at 37° C. under shaking for 10 min. The sample was centrifuged at room temperature for 10 min at 10,000×g. 75 μl of the supernatant were mixed with 75 μl of the SEC mobile phase (20 mM NaH₂PO₄, 140 mM NaCl, 1% SDS, pH 7.4) and 100 μl of the mixture immediately analyzed by SEC.

The resulting size exclusion chromatogram (FIG. 2C) showed peaks at ˜8 ml, ˜19 ml and ˜21 ml elution volume. SDS-PAGE analysis (FIG. 3, lanes 8 and 9) revealed that the peaks at ˜8 ml and ˜19 ml contained most of the peptide. The ˜21 ml elution volume peak again did not contain any detectable Aβ(20-42) peptide.

This indicates that some Aβ(20-42) HMW oligomer had already formed after 10 min of incubation. Nevertheless, the oligomer formation was not yet complete as still some peptide was found in the ˜19 ml elution volume peak.

d) Aβ(20-42) Peptide Sample in Buffer 0.25% SDS for 2 h at 37° C. with Close Tube Lid:

250 μL buffer composed of 5 mM NaH₂PO₄, 35 mM NaCl, 0.25% SDS, pH 7.4, were added to 12.5 μl of the Aβ(20-42) HFIP solution (20 mg/mL) and the sample was incubated at 37° C. under shaking for 10 min. The sample was centrifuged at room temperature for 10 min at 10,000×g. The supernatant was incubated for 2 h with closed tube lid at 850 rpm in an orbital shaker at 37° C. 75 μl of the sample were mixed with 75 μl of the SEC mobile phase (20 mM NaH₂PO₄, 140 mM NaCl, 1% SDS, pH 7.4) and 100 μl of the mixture immediately analyzed by SEC.

The resulting size exclusion chromatogram (FIG. 2D) showed peaks at ˜8 ml, ˜19 ml and ˜21 ml elution volume. SDS-PAGE analysis (FIG. 3, lanes 10 and 11) revealed that the peak at ˜8 ml contained most of the peptide. The peaks at ˜19 ml and ˜21 ml elution volume did not contain any detectable Aβ(20-42) peptide.

This indicates that more Aβ(20-42) HMW oligomer had formed than in example 13c. The Aβ(20-42) HMW oligomer formation was complete as a longer incubation did not lead to a higher yield of A1320-42 HMW oligomer (example 13f).

e) Aβ(20-42) Peptide Sample in Buffer 0.25% SDS for 2 h at 37° C. with Open Tube Lid:

250 μL buffer composed of 5 mM NaH₂PO₄, 35 mM NaCl, 0.25% SDS, pH 7.4, were added to 12.5 μl of the Aβ(20-42) HFIP solution (20 mg/mL) and the sample was incubated at 37° C. under shaking for 10 min. The sample was centrifuged at room temperature for 10 min at 10,000×g. Supernatant was incubated for 2 h with open tube lid at 850 rpm in an orbital shaker at 37° C. 75 μl of the sample were mixed with 75 μl of the SEC mobile phase (20 mM

### SDS-Sample Buffer:

0.3 g SDS
4 mL 1 M Tris/HCl pH 6.8
8 mL glycerol
70 μL 1% bromphenolblue in ethanol
add H₂O to 50 mL

### Running Buffer:

7.5 g Tris
36 g Glycine
2.5 g SDS
add H₂O to 2.5 L

### SDS-PAGE Gel System:

18% Tris/Glycine Gel: (Invitrogen Inc., Cat. no.: EC65055BOX)

### MW-Protein Standard:

SeeBlue Pre-Stained Standard (1×): (Invitrogen Inc., Cat. no.: LC5625)

### Aβ(20-42) Standards:

1 mg of Aβ(20-42) solid powder was suspended in 50 μl 1,1,1,3,3,3-hexafluor-2-propanol and shaked for 1 h at 37° C. to get a clear solution with a concentration of 20 mg/ml. Then 1 μl of this solution were diluted with 799 μl of SDS-sample buffer. The peptide concentration was 25 ng/μl. 20 μl (500 ng Aβ(20-42)) were loaded onto the gel (FIG. 3, lane 2). 20 μL of 25 ng/μl Aβ(20-42) in SDS-sample buffer were diluted with 80 μl SDS-sample buffer. The Peptide concentration was 5 ng/μl. 20 μl (100 ng Aβ(20-42)) were loaded onto the gel (FIG. 3, lane 3).

### SEC Fractions:

10 μl of the SEC fractions of the several runs were diluted with 10 μl SDS-sample buffer. The resulting samples were loaded onto a 18% Tris/Glycin Gel (Invitrogen Inc., Cat. no.: EC65055BOX). The SDS-PAGE was conducted at a constant current of 20 mA.

### Silver Staining:

### Reagent List:

1. Ethanol
2. Acetic Acid
3. Methanol
4. Formaldehyde Solution (30%)
5. Na₂S₂O₃
6. AgNO₃
7. Na₂CO₃

### Reagent Preparation:

Fixing Solution/Stop Solution: ethanol (50% (v/v)), acetic acid (12% (v/v)), water (38% (v/v))

Wash buffer: 50% ethanol in water
Sensitizing Solution: 0.2 g Na₂S₂O₃ in 500 ml water
Staining Solution: 0.4 g AgNO₃+100 μl formaldehyde solution (30%) in 200 ml water
Developing Solution: 12.0 g Na₂CO₃+100 μl formaldehyde solution (30%) in 200 ml water
Destaining Solution: methanol (15% (v/v)), acetic acid (5% (v/v)), water (80% (v/v))

Procedure:

| Step | Reagents | Incubation Time | |
|---|---|---|---|
| fix | Fixing Solution | 1 | hour |
| wash | 50% (v/v) ethanol/water | 3 × 20 | min. |
| sensitize | Sensitizing Solution | 1 | min. |
| wash | dd-H₂O | 3 × 20 | sec. |
| stain | Staining Solution | 20 | min. |
| wash | dd-H₂O | 3 × 20 | sec. |
| develop | Developing Solution | until appropriate band intensity | |
| | | is achieved (3-10 min) | |
| wash | water | 3 × 20 | sec. |
| stop | Stop Solution | 15 | min |
| destain | Destaining Solution | 20 | min |

The sensitizing, staining and developing solutions were prepared immediately before use.

### Example 14

### Aβ(20-42) HMW Oligomer Formation with Different Initial HFIP Concentrations

2.63 mg of Aβ(20-42) peptide were dissolved in 131 μl of 1,1,1,3,3,3-hexafluoro-2-propanol (HFIP) by shaking for 1 h at 37° C. to obtain a clear solution with a concentration of 20 mg/mL Aβ(20-42) peptide (hereinafter referred to as Aβ(20-42) HFIP solution (20 mg/mL)).

### a) 5% HFIP:

95 μL buffer composed of 5 mM NaH₂PO₄, 35 mM NaCl, 0.25% SDS, pH 7.4, were added to 5 μl of the Aβ(20-42) HFIP solution (20 mg/mL) and mixed by vortexing. This results in a 1 mg/ml Aβ(20-42) solution with 5% HFIP. The sample was shaken for 10 min at 37° C. and then centrifuged at room temperature for 10 min at 10,000×g. The supernatant was withdrawn and quiescently incubated for 20 h at room temperature. The sample was again centrifuged at room temperature for 10 min at 10,000×g. The supernatant was withdrawn and 75 μl of the supernatant were mixed with 75 μl of the SEC mobile phase (20 mM NaH₂PO₄, 140 mM NaCl, 1% SDS, pH 7.4) and 100 μl of the mixture immediately analyzed by SEC.

### b) 10% HFIP:

95 μL buffer composed of 90 μl 5 mM NaH₂PO₄, 35 mM NaCl, 0.25% SDS, pH 7.4 and 5 μl HFIP were added to 5 μl of the Aβ(20-42) HFIP solution (20 mg/mL) and mixed by vortexing. This results in a 1 mg/ml Aβ(20-42) solution with 10% HFIP. The sample was shaken for 10 min at 37° C. and then centrifuged at room temperature for 10 min at 10,000×g. The supernatant was withdrawn and quiescently incubated for 20 h at room temperature. The sample was again centrifuged at room temperature for 10 min at 10,000×g. The supernatant was withdrawn and 75 μl of the supernatant were mixed with 75 μl of the SEC mobile phase (20 mM NaH₂PO₄, 140 mM NaCl, 1% SDS, pH 7.4) and 100 μl of the mixture immediately analyzed by SEC.

### c) 25% HFIP:

95 μL buffer composed of 75 μl 5 mM NaH₂PO₄, 35 mM NaCl, 0.25% SDS, pH 7.4 and 20 μl HFIP were added to 5 μl of the Aβ(20-42) HFIP solution (20 mg/mL) and mixed by vortexing. This results in a 1 mg/ml Aβ(20-42) solution with 25% HFIP. The sample was shaken for 10 min at 37° C. and then centrifuged at room temperature for 10 min at 10,000×g. The supernatant was withdrawn and quiescently incubated for 20 h at room temperature. The sample was again centrifuged at room temperature for 10 min at 10,000×g. The supernatant was withdrawn and 75 μl of the supernatant mixed with 75 μl of the SEC mobile phase (20 mM NaH₂PO₄, 140 mM NaCl, 1% SDS, pH 7.4) and 100 μl of the mixture immediately analyzed by SEC.

### d) 50% HFIP:

95 μL buffer composed of 50 μl 5 mM NaH₂PO₄, 35 mM NaCl, 0.25% SDS, pH 7.4 and 45 μl HFIP were added to 5 μl of the Aβ(20-42) HFIP solution (20 mg/mL) and mixed by vortexing. This results in a 1 mg/ml Aβ(20-42) solution with 50% HFIP. The sample was shaken for 10 min at 37° C. and then centrifuged at room temperature for 10 min at 10,000×g. The supernatant was withdrawn and quiescently incubated for 20 h at room temperature. The sample was again centrifuged at room temperature for 10 min at 10,000×g. The supernatant was withdrawn and 75 μl of the supernatant were mixed with 75 μl of the SEC mobile phase (20 mM NaH₂PO₄, 140 mM NaCl, 1% SDS, pH 7.4) and 100 μl of the mixture immediately analyzed by SEC.

SEC analysis was performed as described in reference example 12.

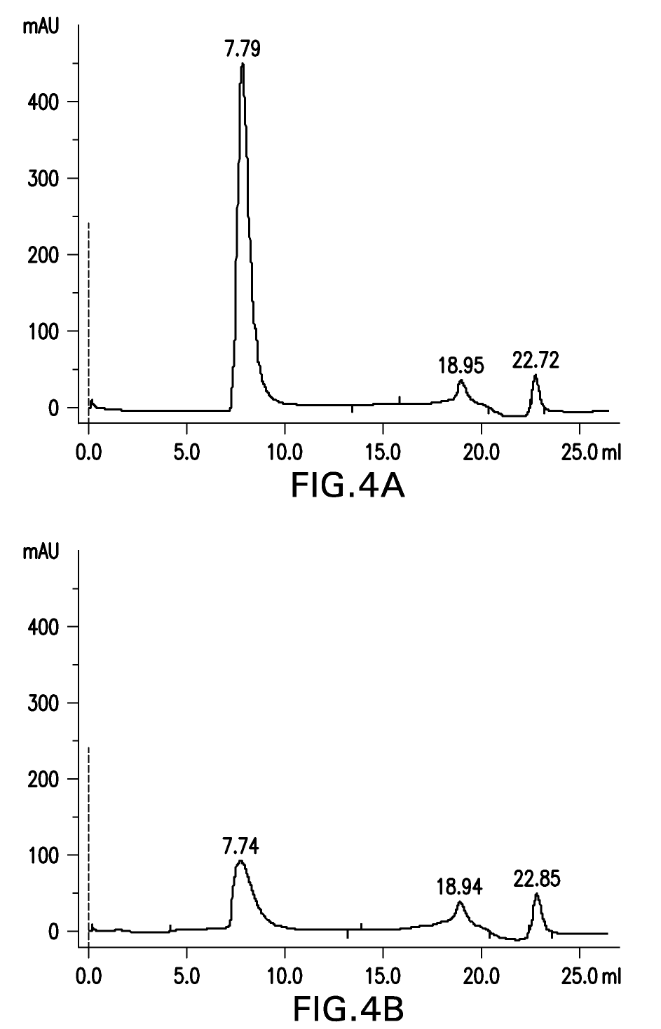

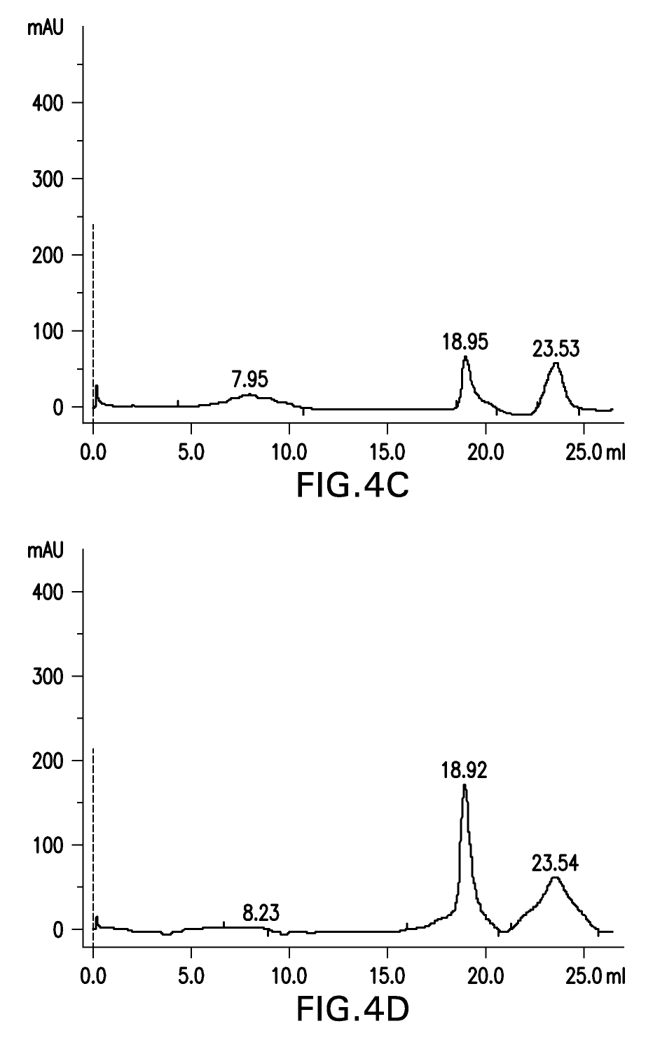

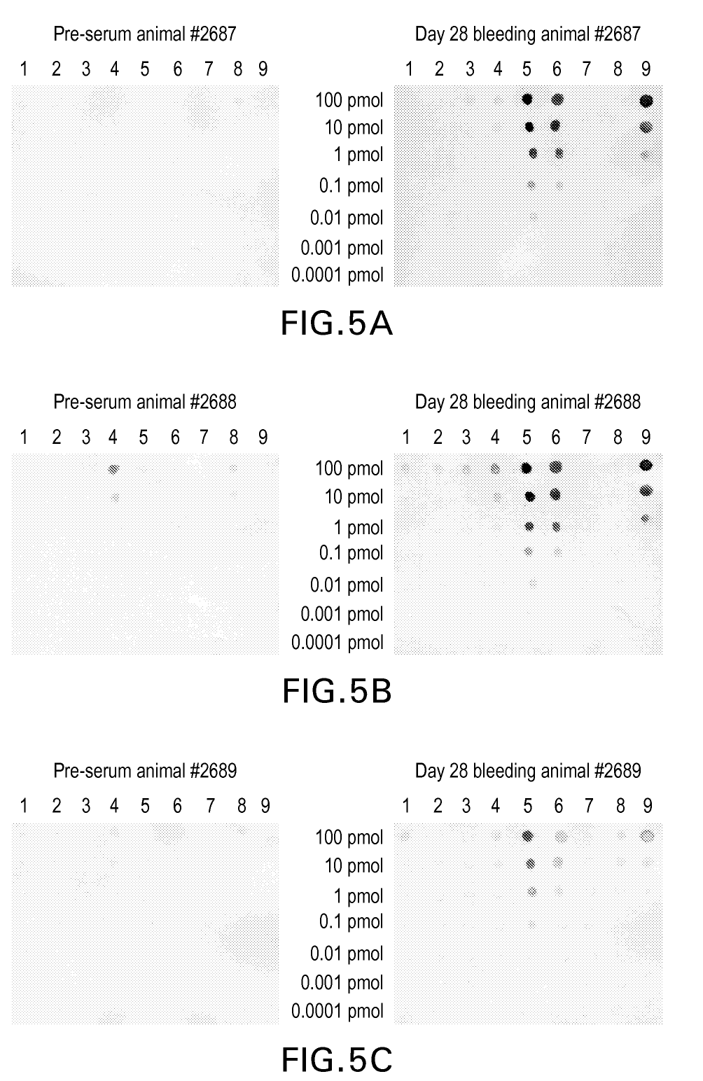

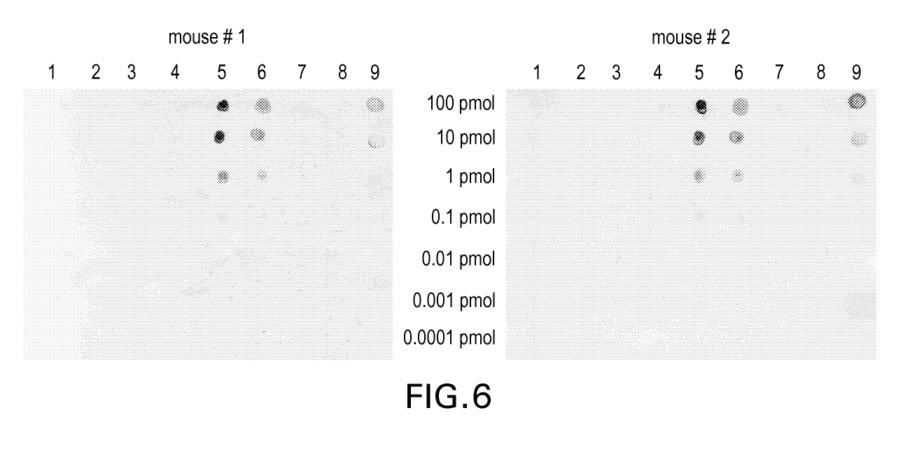

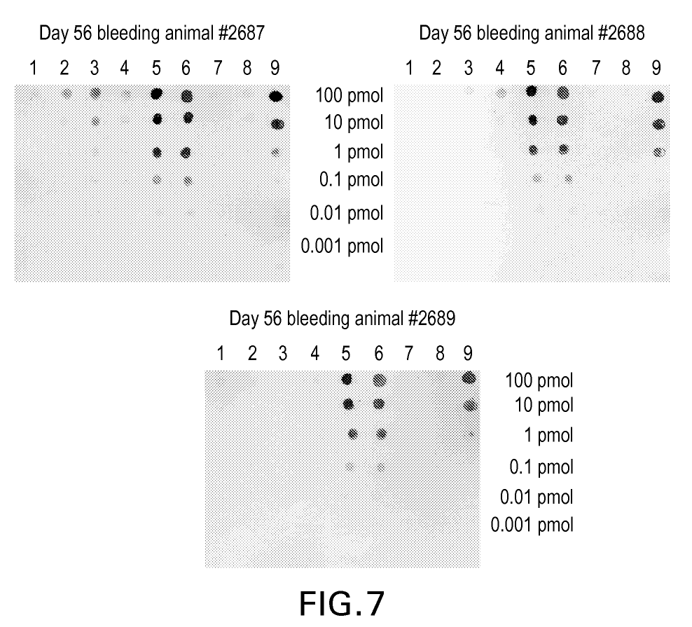

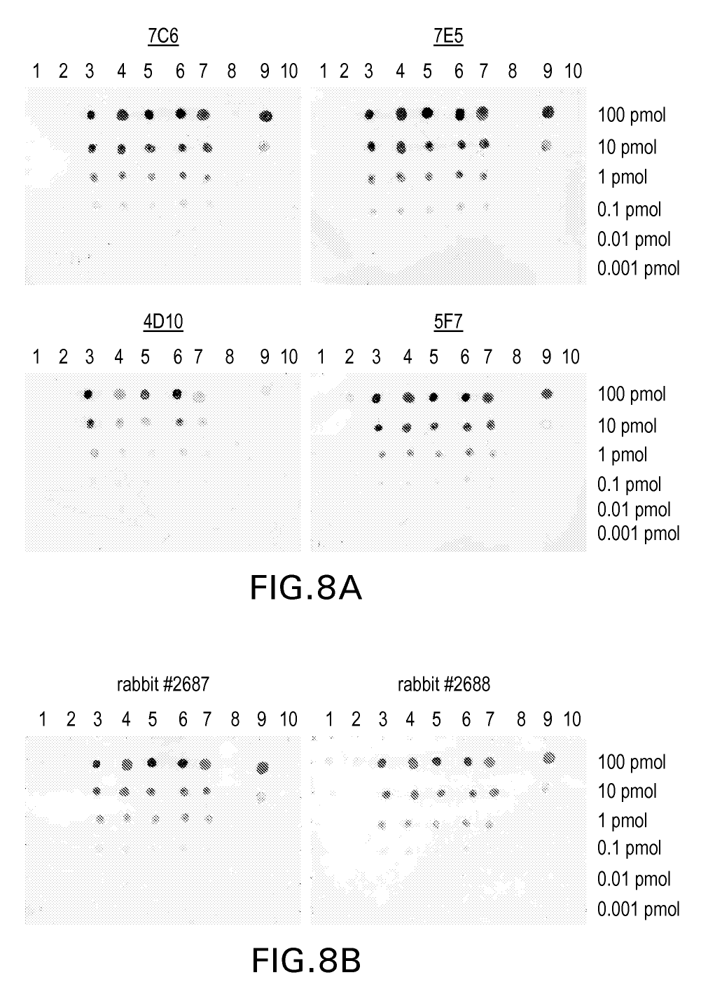

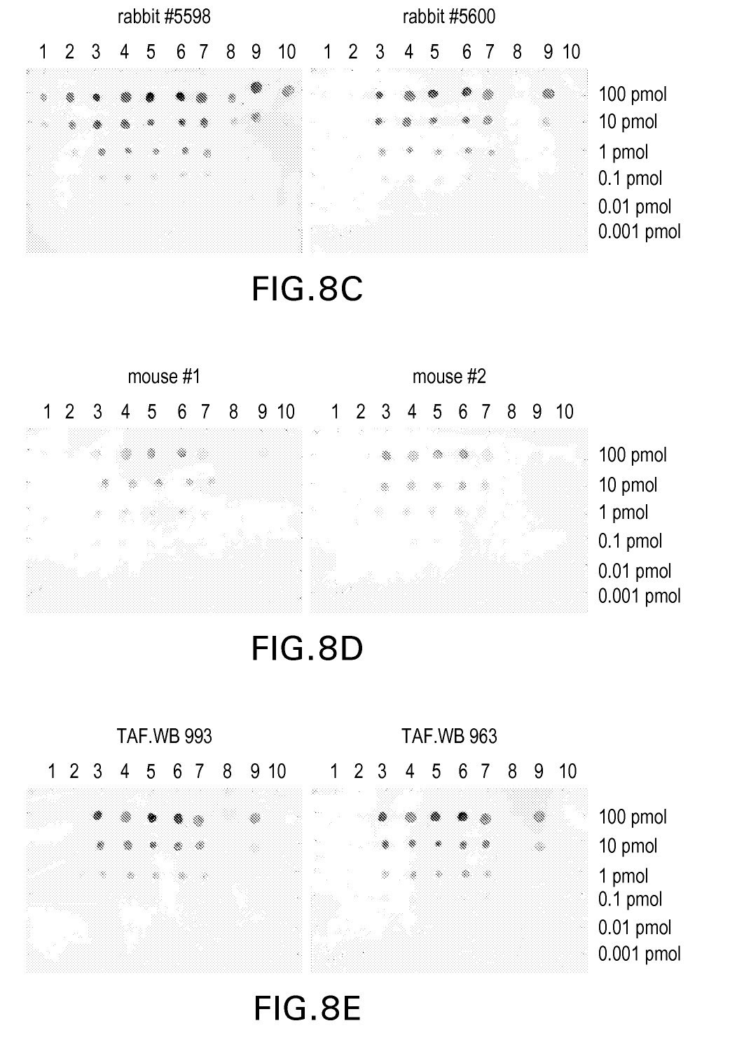

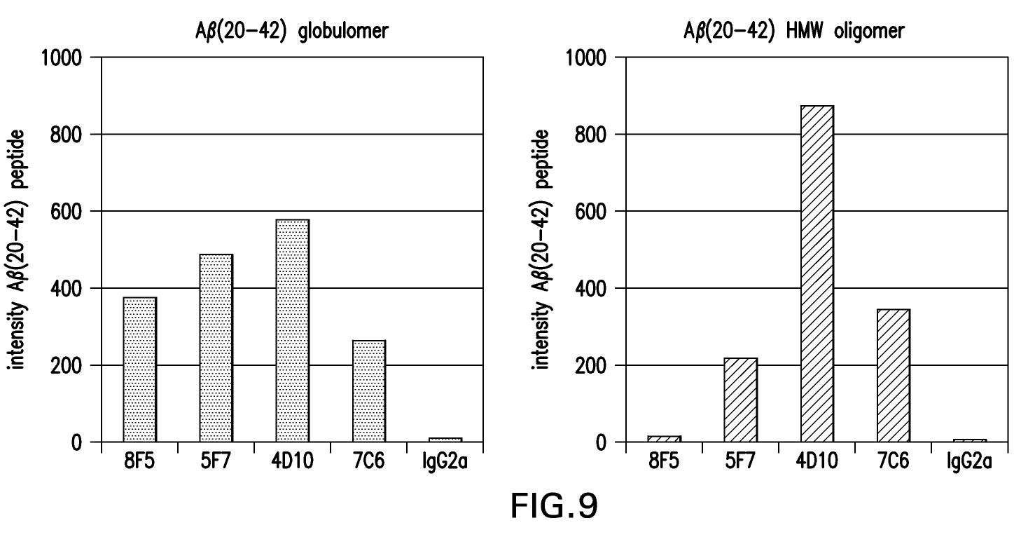

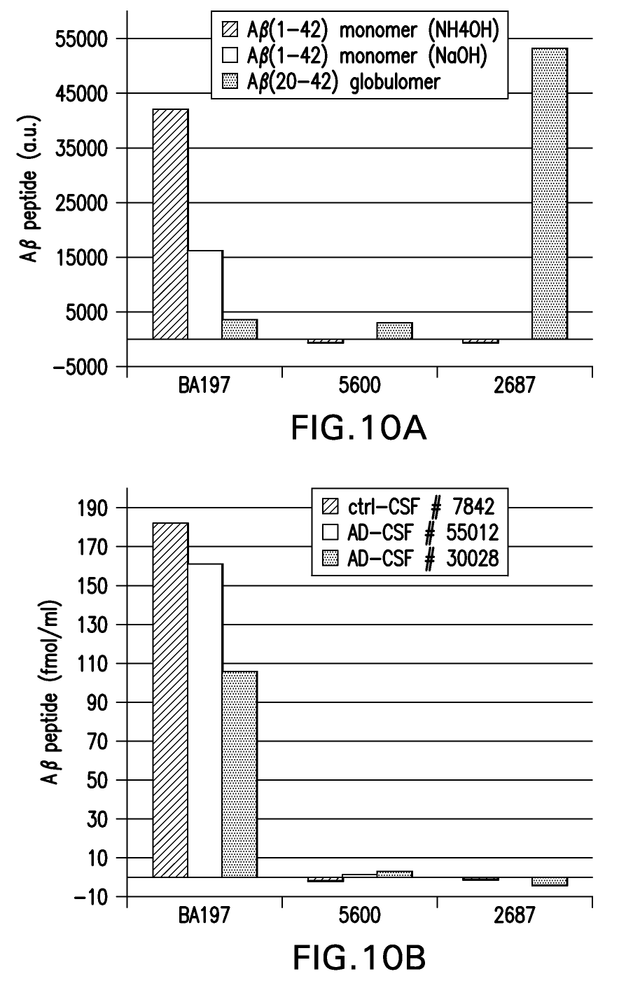

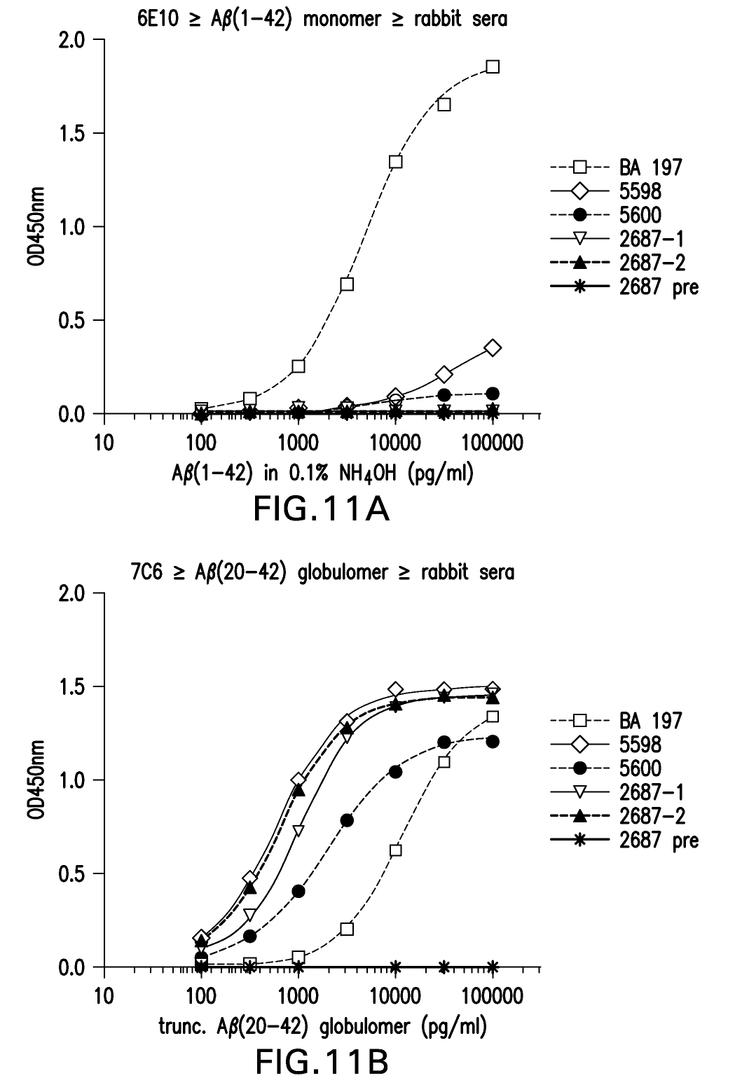

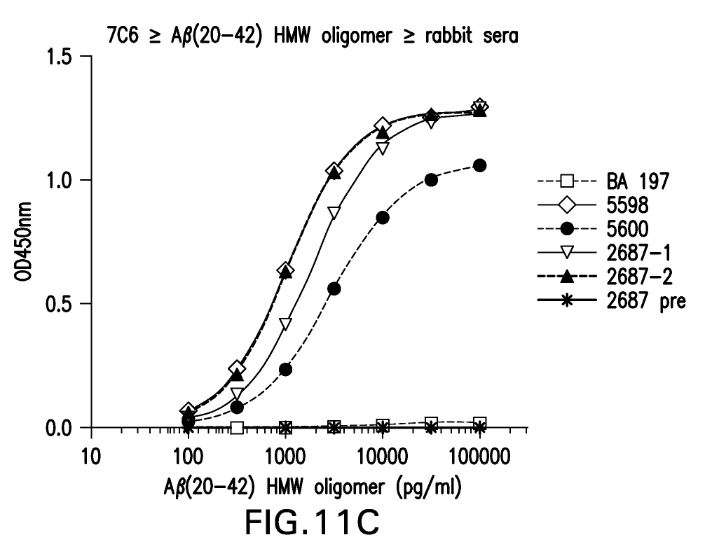

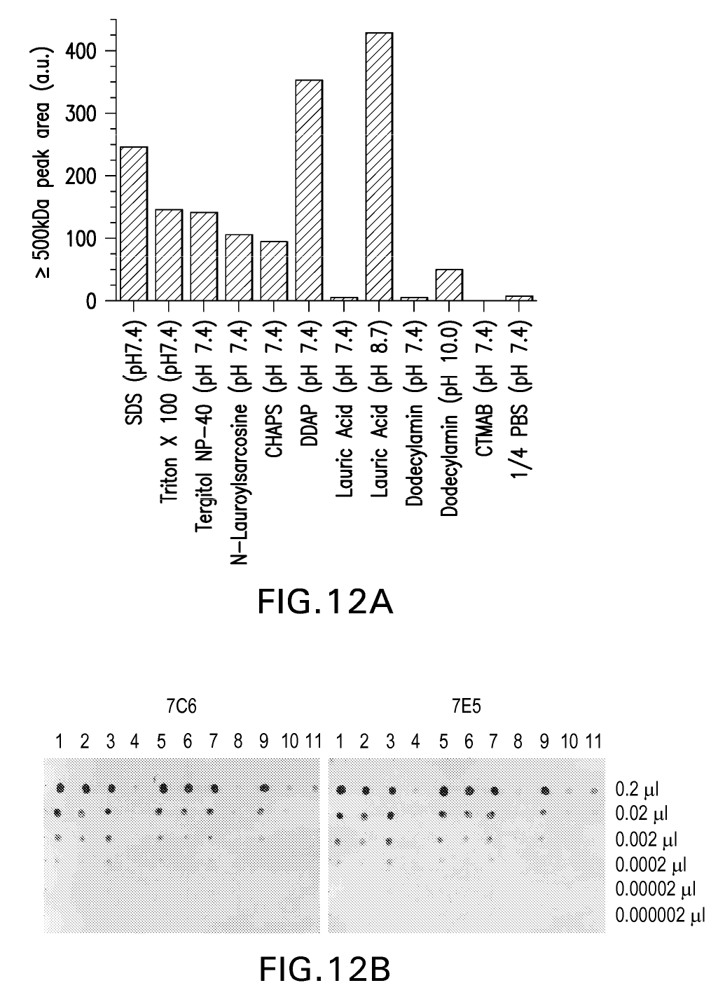

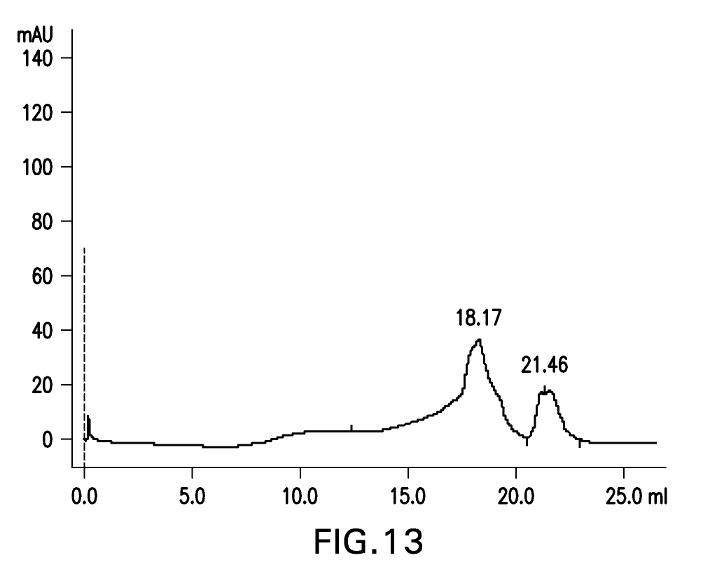

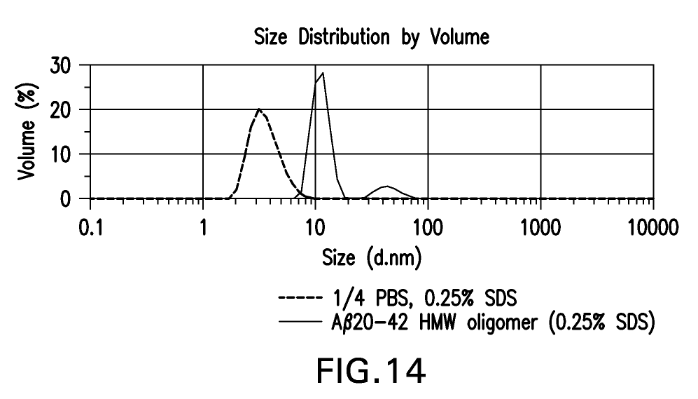

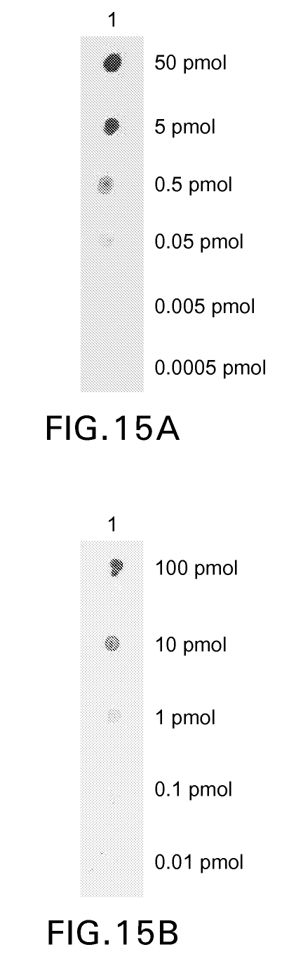

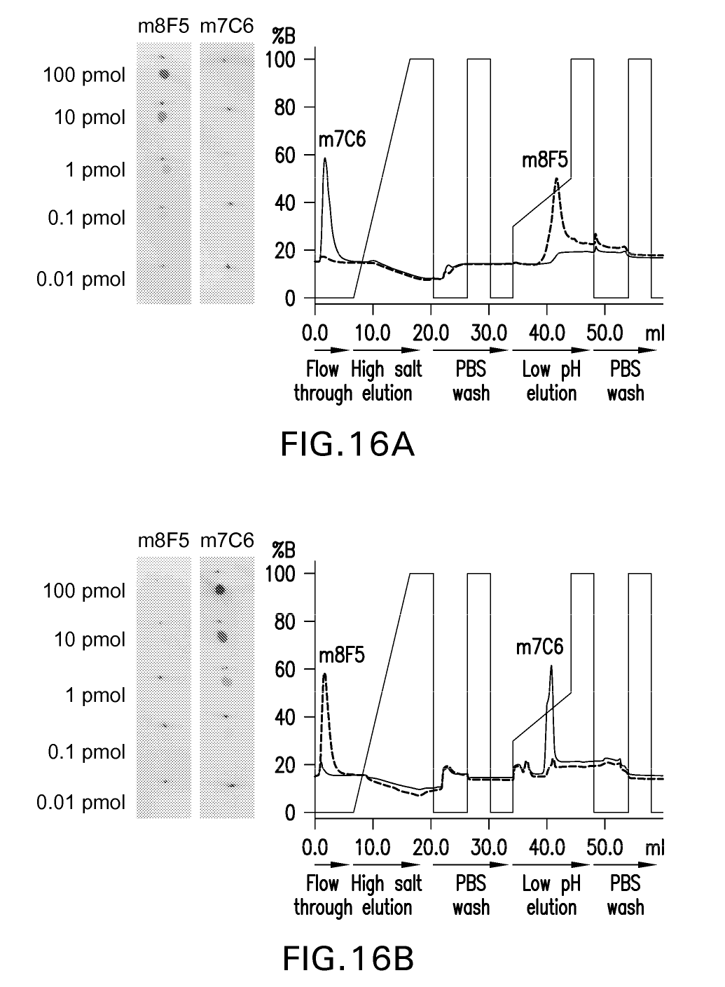

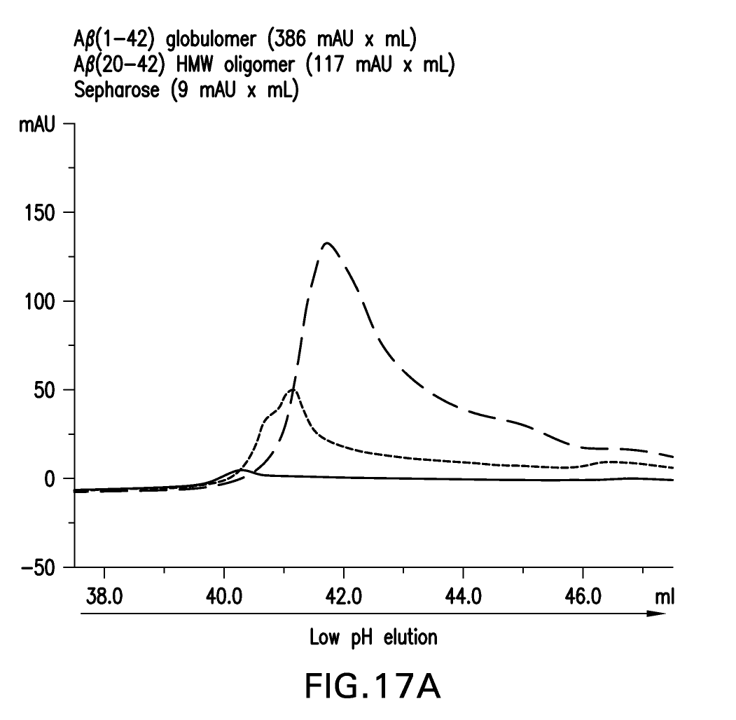

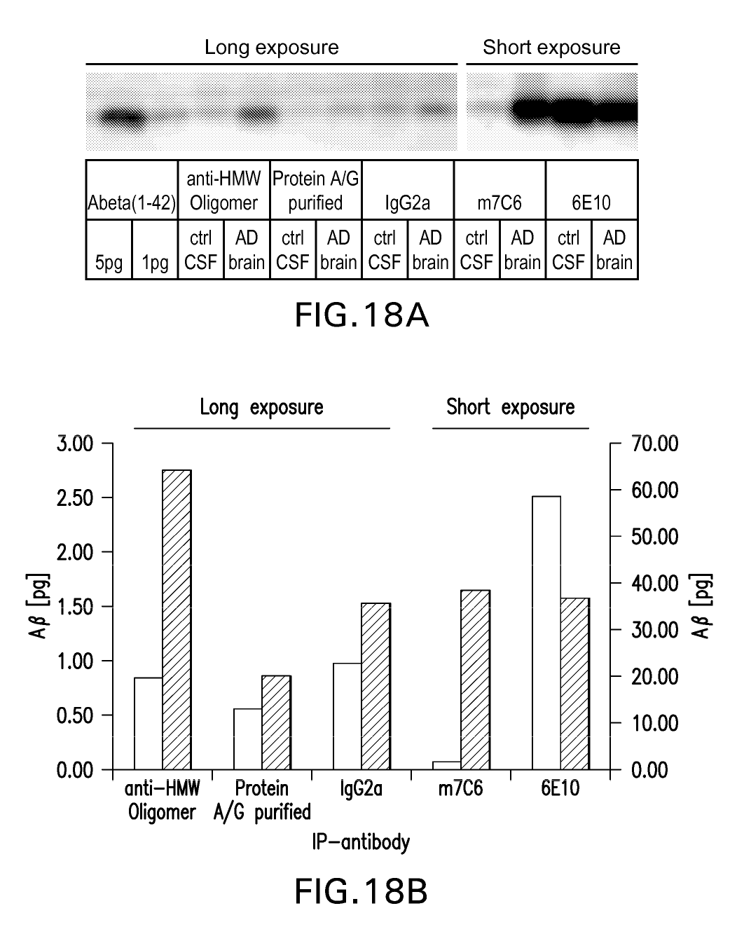

The results are shown in FIG. 4. HFIP as a hydrogen bond breaking agent is used to initially solubilize the Aβ(20-42) peptide and unfold any preexisting secondary or tertiary peptide structures. In case of 5% HFIP in the oligomerization buffer the oligomerization of Aβ(20-42) peptide to Aβ(20-42) HMW oligomers is nearly complete whereas with 10% HFIP a marked reduction of Aβ(20-42) HMW oligomer formation occurs. With 25% HFIP and 50% HFIP Aβ(20-42) HMW oligomer formation is quantitatively almost completely prevented.

### Example 15

### Generation of Aβ(20-42) HMW Oligomer with 0.1% SDS at 6° C.

Aβ(20-42) peptide was obtained via peptide synthesis. 1 mg of the Aβ(20-42) peptide were dissolved in 50 μl 1,1,1,3,3,3,-hexafluoro-2-propanol (HFIP) by shaking for 1 h at 37° C. followed by 10 min centrifugation at 10,000 g. The supernatant is removed yielding a 20 mg/mL Aβ(20-42) peptide stock solution. To 4 μl of this solution 196 μl of buffer composed 2 mM NaH₂PO₄, 14 mM NaCl, pH 7.4 and 20 μl of a 1.0% SDS solution were added and mixed by vortexing (final SDS concentration=0.1%). The sample was incubated for 20 h at 6° C. The sample was centrifuged at room temperature for 10 min at 10,000×g. The supernatant was withdrawn. 75 μl of the supernatant were mixed with 75 μl of the SEC mobile phase (20 mM NaH₂PO₄, 140 mM NaCl, 1% SDS, pH 7.4) and 100 μl of the mixture immediately analyzed by SEC.

The result of the SEC analysis is shown in FIG. 13.

### Biochemical Characterization of Aβ(X-38 . . . 43) Hmw Oligomer

### Example 16

### Aβ(X-38 . . . 43) HMW Oligomer Contains Residual SDS

The residual SDS concentration in the Aβ(20-42) HMW oligomer preparations of examples 1 to 3 was determined via thin layer chromatography against an SDS standard.

### Material List:

Laboratory hood
Blow drier
Compartment drier, temperature 170° C.
4 Development chambers
HPTLC-plates Nano SILGUR-20 (Macherey-Nagel; cat.no. 811032)
SDS-Standards: serial dilution of SDS in 20 mM H₂PO₄, 140 mM NaCl, pH 7.4 (=PBS)
   0.125% SDS in PBS
   0.25% SDS in PBS
   0.5% SDS in PBS
   1% SDS in PBS
   2% SDS in PBS
Aβ(20-42) HMW oligomer sample: serial dilutions in 20 mM H₂PO₄ 140 mM NaCl pH 7.4 (═PBS)
   Directly
   1:2 dilution
   1:4 dilution
   1:8 dilution

### Reagent Preparation:

Mobile Phase 1:
   266 ml Methyl acetate
   266 ml 1-Propanol
   266 ml Chloroform
   96 ml 0.25% KCl-Solution
Mobile Phase 2:
   750 ml n-Hexane
   230 ml Diethylether
   20 ml Acetic Acid
Mobile Phase 3:
   1000 ml n-Hexane
Staining Solution:
   100 ml 85% phosphoric acid
   75 g CuSO₄×5 H₂O
   900 ml H₂O

### Procedure:

Mobile phases 1 to 3 were each filled in 1 development chamber, about 1 cm high, the chambers were closed with a lid and the atmosphere was saturated for at least 20 min.
The staining solution was filled in the development chamber 4 so that the chamber was almost full and allowed the TLC plate to be completely submerged.
The TLC plate was pre-developed in mobile phase 1 for 40 min, dried for 1 min with the blow drier and activated in the compartment drier for 5 min at 170° C.
The sample distances and the developmental height (40 mm above the sample origin) were marked with a pencil on the plate, the sample origin should be above the mobile phase level (about 2 cm high).
The samples were applied to the plate.
The plate was developed with mobile phase 1 up to the marked height (8-12 min)
Then it was dried for 1 min with the blow drier.
The plate was developed with mobile phase 2 up to the top of the plate (6-10 min) and again dried for 1 min with the blow drier.
Afterwards it was developed with mobile phase 3 up to the top of the plate (6-10 min) and dried for 1 min with the blow drier.
The plate was submerged into the staining solution for 2 s and the residual solution was allowed to drip off.
The plate was heated for 12 to 20 min at 170° C. until the staining was sufficient.
Quantitative evaluation was done using a densitometric analysis (GS800 densitometer (BioRad) and software package Quantity one, Version 4.5.0 (BioRad)) of the spot-intensity. Only the spots lying within the SDS standard curve were evaluated.

The residual SDS concentration in different HMW oligomer preparations is shown in table 1.

TABLE 1

| | Oligomer | Oligomer | SDS | | ratio SDS/Aβ |
|---|---|---|---|---|---|
| Example | [mg/ml] | [mM] | % | SDS [mM] | peptide |
| 1 | 1.60 | 0.72 | 1.1 | 38.1 | 52.9 |
| 2 | 0.76 | 0.34 | 0.91 | 31.6 | 92.9 |
| 3 | 1.29 | 0.58 | 0.18 | 6.2 | 10.7 |

The analysis showed that prior dilution with buffer below the critical micellar concentration of SDS (CMC of SDS=0.23%) and subsequent concentration via ultrafiltration can reduce the SDS concentration in relation to the Aβ(20-42) HMW oligomer concentration.

### Example 17

### Aβ(20-42) HMW oligomer and Aβ(22-42) HMW Oligomer are Composed of Aβ(20-42) Peptides and Aβ(22-42) Peptides, Respectively

In order to analyze whether the Aβ(20-42) HMW oligomer are composed of their initial Aβ20-42 peptide sequence or any chemical modification has occurred during the oligomer formation the Aβ(20-42) HMW oligomers were analyzed by Surfac Enhanced Laser Desorption lonuzation Mass Spectrometry (SELDI-MS; BioRad, Protein chip SELDI-system enterprise edition).

To this end:
1 μL Aβ(20-42) HMW oligomer sample was 1:5000-fold diluted with 50% acetonitrile/0.5% TFA. The diluted sample was spotted onto a H4 Protein Chip Array (BioRad; Cat.no. C573-0028);
the spots were allowed to dry on a warm incubator plate;
CHCA-solution:
   5 mg CHCA were dissolved in 150 μL acetonitrile+150 μL 1% TFA=stock solution; stored at −20° C.;
   10 μL of the stock solution were diluted with 20 μL acetonitrile and 20 μL 1% TFA=working CHCA-solution;
   2 μL of the working CHCA-solution were applied onto the spots;
the spots were allowed to dry on a warm incubator plate and analysed by SELDI-MS;
   BioRad SELDI-MS SELDI-MS; BioRad, Protein chip SELDI-system enterprise edition) instrument settings: mass range: 0 to 10000 Da; focus mass: 4500 Da; matrix attenuation: 500 Da; sampling rate: 400 MHz; warming shots: 2 with energy: 1100 nj; data shots: 10 with energy 800-1000 nJoule; Partition 1 of 3.

SELDI-MS analysis showed that the Aβ(20-42) HMW oligomer and the Aβ(22-42) HMW oligomer are composed of their initial Aβ peptide sequence. No modification, degradation or cross linking appears to have occurred during its production (table 3).

TABLE 2

| | initial | amphipathic | peptide mass | peptide mass |
|---|---|---|---|---|
| Example | solvent | agent | (theor.) [kDa] | (determ.) [kDa] |
| 1 | HFIP | SDS | 2217.6 | 2216.9 |
| 2 | HFIP | SDS | 2217.6 | 2218.9 |
| 3 | HFIP | SDS | 2217.6 | 2219.1 |
| 4 | HFIP | Lauric acid | 2217.6 | 2219.1 |
| 5 | HFIP | N- | 2217.6 | 2219.8 |
| | | Lauroylsarcosine | | |
| 6 | HFIP | SDS | 1999.3 | 2000.2 |
| 7 | 0.1% | SDS | 2217.6 | 2219.5 |
| | NaOH | | | |
| 8 | 0.1% | N- | 2217.6 | 2219.4 |
| | NaOH | Lauroylsarcosine | | |

### Example 18

### Photon correlation spectroscopy of Aβ(20-42) HMW oligomer

For extended characterization purposes, the particle size (hydrodynamic radius) of the Aβ(20-42) HMW oligomer was determined at 25° C. via Photon Correlation Spectroscopy using a backscatter angle of 173° calculating the average of 3×10 seconds scans. The figure depicts the calculated size distribution per volume assuming viscosity values of 1.0 cP.

Photon correlation spectroscopy showed that Aβ(20-42) HMW oligomer (0.25% SDS from example 1) had a size of ˜10-20 nm (major species) and 50-100 nm (minor species) and could clearly be distinguished from SDS micelles with ˜4 nm in size (FIG. 14).

### Biological Characterization of Aβ(X-38 . . . 43) HMW Oligomer

### Example 19

### Aβ(20-42) HMW Oligomer Induces Aβ(20-42) Oligomer Specific Immune Response in Rabbits and Mice

Dot blot analysis of anti-Aβ antibodies produced after active immunization of mice and rabbits with Aβ(20-42) HMW oligomer was performed to assess their specificity towards different forms of Aβ. The individual forms of Aβ were blotted in serial dilutions and incubated with the corresponding mouse plasma and rabbit sera containing anti-Aβ antibodies produced during immune reaction. The individual dot blots correspond to different individuals of immunized rabbits or mice.

a) Active immunization of rabbits # 2687, #2689 and #2689 with Aβ(20-42) HMW oligomer The rabbits received 200 μg of Aβ(20-42) HMW oligomer prepared according to example 1 mixed with BioGenes-Adjuvant (BioGenes GmbH, Berlin, Germany) intraperitoneally (initial immunization at day 0), followed by 2 booster injections at day 14 and day 21 with 100 μg of Aβ(20-42) HMW oligomer prepared according to example 1 mixed with BioGenes-Adjuvant (BioGenes GmbH, Berlin, Germany) intraperitoneally. The first bleeding was 28 days after the initial immunization. After one further boost at day 49 the second bleeding was 56 days after the initial immunization.
b) Active Immunization of Mice #1 and #2 with Aβ(20-42) Hmw Oligomer

The mice (non-transgenic littermates of Tg2576 mice) received 30 μg of Aβ(20-42) HMW oligomer prepared according to example 2 mixed with an equal amount of complete Freund's adjuvant intraperitoneally.

After immunization rabbit and mouse plasma samples were assessed for anti-Aβ antibodies. To this end, dilution series of the individual Aβ(1-42) and Aβ(20-42) forms ranging from 100 pmol/μl to 0.0001 μmol/μl in PBS supplemented with 0.2 mg/ml BSA were made. 1 μl of each sample was blotted onto a nitrocellulose membrane. For detection the corresponding rabbit and mouse plasma samples were used (diluted 1:200). Immunostaining was done using alkaline phosphatase conjugated anti-mouse-IgG or anti-rabbit-IgG respectively and the staining reagent NBT/BCIP.

### Method:

### Aβ Standards for Dot Blot:

1. Aβ(1-40) monomer (0.1% NaOH)

The preparation of Aβ(1-40) monomer (0.1% NaOH) is described in reference example 1.

2. Aβ(1-42) monomer (0.1% NaOH)

The preparation of Aβ(1-42) monomer (0.1% NaOH) is described in reference example 2.

3. Aβ(1-42) globulomer

The preparation of Aβ(1-42) globulomer is described in reference example 3.

4. Aβ(12-42) globulomer

The preparation of Aβ(12-42) globulomer is described in reference example 4.

5. Aβ(20-42) globulomer

The preparation of Aβ(20-42) globulomer is described in reference example 5.

6. Aβ(20-42) HMW oligomer

The preparation of the Aβ(20-42) HMW oligomer is described in example 1.

7. Aβ fibrils

The preparation of Aβ fibrils is described in reference example 6.

8. sAPPα

The preparation of sAPP (is described in reference example 7.

9. Aβ(20-42) monomer, 50% HFIP

The preparation of Aβ(20-42) monomer, 50% HFIP is described in reference example 8.

### Materials for Dot Blot:

### Aβ Standards:

Serial dilution of Aβ antigens in columns 1-7 in 20 mM NaH2PO4, 140 mM NaCl, pH 7.4+0.2 mg/ml BSA
   1) 100 μmol/μl
   2) 10 μmol/μl
   3) 1 μmol/μl
   4) 0.1 μmol/μl
   5) 0.01 μmol/μl
   6) 0.001 μmol/μl
   7) 0.0001 μmol/μl
Serial dilution of Aβ antigens in column 8 in 20 mM NaH₂PO₄, 140 mM NaCl, pH 7.4+0.2 mg/ml BSA
   1) 1 μmol/μl
   2) 0.1 μmol/μl
   3) 0.01 μmol/μl
   4) 0.001 μmol/μl
   5) 0.0001 μmol/μl
   6) 0.00001 μmol/μl
   7) 0.000001 μmol/μl
Serial dilution of Aβ antigens in column 9 in 50% HFIP in water
   1) 100 μmol/μl
   2) 10 μmol/μl
   3) 1 μmol/μl
   4) 0.1 μmol/μl
   5) 0.01 μmol/μl
   6) 0.001 μmol/μl
   7) 0.0001 μmol/μl

### Nitrocellulose:

Trans-Blot Transfer medium, Pure Nitrocellulose Membrane (0.45 μm); BioRad

### Anti-Mouse-AP:

AP326A (Chemicon)

### Anti-Rabbit-AP:

AP304A (Chemicon)

### Detection Reagent:

NBT/BCIP Tablets (Roche)

### Bovine Serum Albumin, (BSA):

11926 Serva

### Blocking Reagent:

5% low fat milk in TBS

### Buffer Solutions:

TBS
25 mM Tris/HCl-buffer pH 7.5
+150 mM NaCl
TTBS
25 mM Tris/HCl-buffer pH 7.5
+150 mM NaCl
+0.05% Tween 20
PBS+0.2 mg/ml BSA
20 mM NaH₂PO₄ buffer pH 7.4
+140 mM NaCl
+0.2 mg/ml BSA

### Antibody Solution I:

Mouse plasma samples and rabbit sera samples (1:200 diluted in 20 ml 1% low fat milk in TBS)
Mouse monoclonal antibodies (diluted to 0.2 μg/ml in 20 ml 1% low fat milk in TBS)

### Antibody Solution II:

1:5000 dilution
Anti-Mouse-AP and Anti-Rabbit-AP in 1% low fat milk in TBS

### Dot Blot Procedure:

1) 1 μl each of the different Aβ standards (in their 7 serial dilutions) were dotted onto the nitrocellulose membrane in a distance of approximately 1 cm from each other.
2) The Aβ standards dots were allowed to dry on the nitrocellulose membrane on air for at least 10 min at room temperature (RT) (=dot blot)
3) Blocking:
   The dot blot was incubated with 30 ml 5% low fat milk in TBS for 1.5 h at RT.
4) Washing:
   The blocking solution was discarded and the dot blot was incubated under shaking with 20 ml TTBS for 10 min at RT.
5) Antibody solution I:
   The washing buffer was discarded and the dot blot was incubated with antibody solution I for 2 h at RT.
6) Washing:
   The antibody solution I was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
7) Antibody solution II:
   The washing buffer was discarded and the dot blot was incubated with antibody solution II for 1 h at RT
8) Washing:
   The antibody solution II was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
9) Development:
   The washing solution was discarded. 1 tablet NBT/BCIP was dissolved in 20 ml H₂O and the dot blot was incubated for 4 min with this solution. The development was stopped by intensive washing with H₂O.

The results are shown in FIGS. 5 and 6.

Active immunization with the Aβ(20-42) HMW oligomer elicits an Aβ(20-42) oligomer selective immune response in rabbits (FIG. 5) and mice (FIG. 6). The resulting plasma contains antibodies which recognize:
Aβ(20-42) HMW oligomer
Aβ(20-42) truncated globulomer
to a lower extent monomeric Aβ(20-42) peptide (only at least 10-fold up to 100-fold higher monomeric Aβ(20-42) peptide are recognized compared to the Aβ(20-42) HMW oligomer).

They do not recognize:
Aβ1-40 monomers
Aβ(1-42) monomers
Aβ(1-42) globulomers
Aβ12-42 globulomers (recognition to a low extent)
Aβ fibrils
sAPPα

The selectivity of the immune response in rabbits is maintained even after multiple active immunizations with Aβ(20-42) HMW oligomer (FIG. 7).

### Example 20

### Aβ(20-42) HMW Oligomer is Recognized by Aβ(20-42) Globulomer Specific Antibodies Using the Dot Blot Assay

Different preparations of Aβ(20-42) HMW oligomers were tested for their recognition by various monoclonal antibodies and mouse and rabbit immune sera. To this end, dilution series of the individual Aβ(1-42) and Aβ(20-42) forms ranging from 100 μmol/μl to 0.001 μmol/μl, 50 μmol/μl to 0.0005 μmol/μl, or 100 μmol/μl to 0.01 μmol/μl in PBS supplemented with 0.2 mg/ml BSA were made. 1 μl of each sample was blotted onto a nitrocellulose membrane. For detection the corresponding monoclonal antibodies were used (concentration of 0.2 μg/ml) or polyclonal rabbit and mouse plasma samples were used (diluted 1:200). Immunostaining was done using alkaline phosphatase conjugated anti-mouse-IgG or anti-rabbit-IgG respectively and the staining reagent NBT/BCIP.

### Method:

### Aβ Standards for Dot Blot A:

1. Aβ(1-42) monomer (0.1% NaOH)

The preparation of Aβ(1-42) monomer (0.1% NaOH) is described in reference example 2.

2. Aβ(1-42) globulomer

The preparation of Aβ(1-42) globulomer is described in reference example 3.

3. Aβ(20-42) globulomer

The preparation of Aβ(20-42) globulomer is described in reference example 5.

4. Aβ(20-42) HMW oligomer

The preparation of the Aβ(20-42) HMW oligomer is described in example 2

5. Aβ(20-42) HMW oligomer

The preparation of the Aβ(20-42) HMW oligomer is described in example 3

6. Aβ(20-42) HMW oligomer (Lauric acid)

The preparation of the Aβ(20-42) HMW oligomer is described in example 4

7. Aβ(20-42) HMW oligomer (N-Lauroylsarcosine)

The preparation of the Aβ(20-42) HMW oligomer is described in example 5

8. Aβ(22-42) HMW oligomer

The preparation of the Aβ(22-42) HMW oligomer is described in example 6

9. Aβ(20-42) monomer, 50% HFIP

The preparation of Aβ(20-42) monomer is described in reference example 8.

10. Aβ(22-42) monomer, 50% HFIP

The preparation of Aβ(20-42) monomer is described in reference example 9.

### Aβ Standards for Dot Blot B:

1. Aβ(20-42) peptide+0.5% SDS, (pH 7.4)

This preparation is described in example 12A.

2. Aβ(20-42) peptide+0.2% Triton x 100, (pH 7.4)

This preparation is described in example 12B.

3. Aβ(20-42) peptide+0.2% Tergitol NP-40, (pH 7.4)

This preparation is described in example 12C.

4. Aβ(20-42) peptide+0.5% Lauric Acid (pH 7.4)

This preparation is described in example 12D.

5. Aβ(20-42) peptide+0.5% N-Lauroylsarcosine (pH 7.4)

This preparation is described in example 12E.

6. Aβ(20-42) peptide+0.5% CHAPS (pH 7.4)

This preparation is described in example 12F.

7. Aβ(20-42) peptide+0.25% Dodecyl-N,N-dimethyl-3-amino-1-propane sulfonate (DDAP) (pH 7.4)

This preparation is described in example 12G.

8. Aβ(20-42) peptide+0.5% Dodecylamin (pH 7.4)

This preparation is described in example 12H.

9. Aβ(20-42) peptide+0.5% Lauric Acid (pH 8.7)

This preparation is described in example 12J.

10. Aβ(20-42) peptide+0.5% Dodecylamin (pH 10.0)

This preparation is described in example 12K.

11. Aβ(20-42) peptide+¹/4 PBS (pH 7.4)

This preparation is described in example 12L.

### Aβ Standards for Dot Blot C:

1. Aβ(20-42) HMW oligomer (0.2% Tween 80)

The preparation of the Aβ(20-42) HMW oligomer is described in example 9.

### Aβ Standards for Dot Blot D:

1. Aβ(20-42) HMW oligomer (pH 8.5)

The preparation of Aβ(20-42) HMW oligomer in buffer at pH 8.5 is described in example 10.

### Materials for Dot Blot:

### Aβ Standards for Dot Blot A:

Serial dilution of Aβ antigens in columns 1-8 in 20 mM NaH₂PO₄, 140 mM NaCl, pH 7.4+0.2 mg/ml BSA
   1) 100 pmol/μl
   2) 10 pmol/μl
   3) 1 pmol/μl
   4) 0.1 pmol/μl
   5) 0.01 pmol/μl
   6) 0.001 μmol/μl
Serial dilution of Aβ antigens in columns 9-10 in 50% HFIP in water
   1) 100 pmol/μl
   2) 10 pmol/μl
   3) 1 pmol/μl
   4) 0.1 pmol/μl
   5) 0.01 pmol/μl
   6) 0.001 pmol/μl

### Aβ Standards for Dot Blot B:

Serial dilution of Aβ antigens in columns 1-11 in 20 mM NaH₂PO₄, 140 mM NaCl, pH 7.4+0.2 mg/ml BSA
   1) 0.2 μl/μl
   2) 0.02 μl/μl
   3) 0.002 μl/μl
   4) 0.0002 μl/μl
   5) 0.00002 μl/μl
   6) 0.000002 μl/μl

### Aβ Standards for Dot Blot C:

Serial dilution of Aβ antigens in columns 1-2 in 20 mM NaH₂PO₄, 140 mM NaCl, pH 7.4+0.2 mg/ml BSA
   1) 50 pmol/μl
   2) 5 pmol/μl
   3) 0.5 pmol/μl
   4) 0.05 pmol/μl
   5) 0.005 pmol/μl
   6) 0.0005 pmol/μl

### Aβ Standards for Dot Blot D:

Serial dilution of Aβ antigens in columns 1-2 in 20 mM NaH₂PO₄, 140 mM NaCl, pH 7.4+0.2 mg/ml BSA
   1) 100 pmol/μl
   2) 10 pmol/μl
   4) 0.1 pmol/μl
   5) 0.01 pmol/μl

### Nitrocellulose:

Trans-Blot Transfer medium, Pure Nitrocellulose Membrane (0.45 μm); BioRad

### Anti-Mouse-AP:

AP326A (Chemicon)

### Anti-Rabbit-AP:

AP304A (Chemicon)

### Detection Reagent:

NBT/BCIP Tablets (Roche)

### Bovine Serum Albumin, (BSA):

11926 Serva

### Blocking Reagent:

5% low fat milk in TBS

### Buffer Solutions:

TBS
25 mM Tris/HCl-buffer pH 7.5
+150 mM NaCl
TTBS
25 mM Tris/HCl-buffer pH 7.5
+150 mM NaCl
+0.05% Tween 20
PBS+0.2 mg/ml BSA
20 mM NaH₂PO₄ buffer pH 7.4
+140 mM NaCl
+0.2 mg/ml BSA

### Antibody Solution I:

Mouse plasma samples (1:200 diluted in 5 ml 1% low fat milk in TBS) and rabbit sera samples (1:200 diluted in 20 ml 1% low fat milk in TBS)
Mouse monoclonal antibodies (diluted to 0.2 μg/ml in 20 ml 1% low fat milk in TBS)

### Antibody Solution II:

1:5000 dilution
Anti-Mouse-AP and Anti-Rabbit-AP in 1% low fat milk in TBS

### Dot Blot Procedure:

1) 1 μl each of the different Aβ standards (in their 7, 6 or 5 serial dilutions) were dotted onto the nitrocellulose membrane in a distance of approximately 1 cm from each other.
2) The Aβ standards dots were allowed to dry on the nitrocellulose membrane on air for at least 20 min at room temperature (RT) (=dot blot)
3) Blocking:
   The dot blot was incubated with 30 ml 5% low fat milk in TBS for 1.5 h at RT.
4) Washing:
   The blocking solution was discarded and the dot blot was incubated under shaking with 20 ml TTBS for 10 min at RT.
5) Antibody solution I:
   The washing buffer was discarded and the dot blot was incubated with antibody solution I for 2 h at RT.
6) Washing:
   The antibody solution I was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
7) Antibody Solution II:
   The washing buffer was discarded and the dot blot was incubated with antibody solution II for 1 h at RT
8) Washing:
   The antibody solution II was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
9) Development:
   The washing solution was discarded. 1 tablet NBT/BCIP was dissolved in 20 ml H₂O and the dot blot was incubated for 4 min with this solution. The development was stopped by intensive washing with H₂O.

The results are shown in FIG. 8 (dot blot A), 12B (dot blot B), 15A (dot blot C) and 15 B (dot blot D).

The following monoclonal antibodies and polyclonal mouse or rabbit sera
Aβ globulomer specific antibodies 7C6, 5F7 and 4D10 (raised against the Aβ(20-42) globulomer) (FIG. 8A)
polyclonal antisera from rabbits immunized with Aβ(20-42) HMW oligomer from example 1 (rabbit #2687 and #2688) (FIG. 8B)
polyclonal antisera from rabbits immunized with Aβ(20-42) globulomer (rabbit #5598 and #5600) (FIG. 8C)
polyclonal antisera from mice immunized with Aβ(20-42) HMW oligomer from example 2 (mouse #1 and #2) (FIG. 8D)
polyclonal antisera from APP/Lo mice immunized with Aβ(20-42) globulomer (mouse #TAF.WB 993, #TAF.WB 963) (FIG. 8E)
   have a similar sensitive and selective recognition for the Aβ(20-42) HMW oligomer preparations of examples 2 to 5 compared to the Aβ(20-42) truncated globulomer (7C6 even detected Aβ(20-42) HMW oligomer at an about 10-times lower concentration than Aβ(20-42) truncated globulomer).

Further, the following monoclonal Aβ globulomer specific antibody 7C6 (raised against the Aβ(20-42) globulomer) recognizes the Aβ(20-42) HMW oligomer preparations of example 9 (FIG. 15A) and example 10 (FIG. 15B).

This shows that the different Aβ(20-42) HMW oligomers are comparable from a protein-conformational standpoint as they all possess the Aβ globulomer epitope.

Therefore, it is expected that an active immunization with Aβ(20-42) HMW oligomer will be effective in reversing cognitive deficits in AD transgenic mouse models as the elicited antibody response is comparable in its polyclonal antisera dot blot profile to a polyclonal antisera dot blot profile from an active immunization with Aβ(20-42) truncated globulomer. The latter has been proven to reverse cognitive deficits in an object recognition task.

Furthermore, active immunization with Aβ(20-42) HMW oligomer elicits a highly selective immune response for the Aβ globulomer epitope, which was also shown previously with the Aβ(20-42) truncated globulomer. However, as no N-terminus exists in the Aβ(20-42) HMW oligomer, the remaining risk of eliciting an unspecific N-terminal Aβ peptide directed immune response is eliminated. In contrast, Aβ(20-42) truncated globulomer active immunization has been shown to produce in occasional cases an unspecific immune response (FIG. 8C, rabbit #5598).

The amphipathic agents:
0.5% SDS, (pH 7.4), anionic amphipathic agent
0.2% Triton X100, (pH 7.4), neutral amphipathic agent
0.2% Tergitol NP-40, (pH 7.4), neutral amphipatic agent
0.5% N-Lauroylsarcosine (pH 7.4), anionic amphipathic agent
0.5% CHAPS (pH 7.4), hybrid ionic amphipatic agent
0.25% Dodecyl-N,N-dimethyl-3-amino-1-propansulfonat (DDAP) (pH 7.4), hybrid ionic amphipatic agent
0.5% Lauric Acid (pH 8.7), anionic amphipathic agent
   induced Aβ(20-42) HMW oligomers which are detected in a dot blot assay by the Aβ globulomer specific antibodies 7C6 and 7E5 (raised against the Aβ(20-42) truncated globulomer) down to the same dilution (FIG. 12B). This shows that the different Aβ(20-42) HMW oligomers are comparable from a protein-conformational standpoint as they all posses the Aβ globulomer epitope.

The amphipathic agent:
0.5% Dodecylamin (pH 10.0), cationic amphipathic agent induced Aβ(20-42) HMW oligomers which is detected in a dot blot assay by the Aβ globulomer specific antibodies 7C6 and 7E5 (raised against the Aβ(20-42) truncated globulomer) only at a lower dilution (FIG. 12B). This shows that the Aβ(20-42) HMW oligomer induced by dodecylamin also contains the Aβ globulomer epitope.

The amphipathic agents:
0.5% Lauric Acid (pH 7.4), anionic amphipathic agent
0.5% Dodecylamin (pH 7.4), cationic amphipathic agent
0.2% Cetyltrimethylammoniumbromid (pH 7.4), cationic amphipathic agent
   did not induce substantial amounts of Aβ(20-42) HMW oligomer. If no amphipathic agent is added (in the case of the ¼ PBS sample) no substantial amount of Aβ(20-42) HMW oligomer can be detected dot blot either (FIG. 12B).

### Example 21

### Recognition of Aβ(20-42) HMW Oligomer by Aβ Globulomer Specific Antibodies Using Immunoprecipitation with SELDI-MS Quantification

A: Activation of Dynabeads with Monoclonal Mouse Antibodies
The stock-suspension of dynabeads (Dynabeads M-280 Sheep anti-Mouse IgG, Invitrogen; Cat. no.: 112.02) was shaken carefully to prevent foaming.
1 mL was aseptically removed and transferred to a 1.5 mL reaction vial.
The dynabeads were washed 3 times 5 min with 1 mL immunoprecipitation (IP)-wash buffer (IP-wash-buffer: PBS (20 mM NaH₂PO₄, 140 mM NaCl, pH 7.4), 0.1% BSA). During the washing procedure the supernatant was carefully removed while the dynabeads were immobilized at the side of the reaction vial with a magnetic separator stand (MSS).
The washed dynabeads were incubated with 40 μg Aβ antibody in 1 mL PBS, 0.1% BSA
The activation was carried out by overnight incubation under shaking at 4° C.
The activated dynabeads were washed 4 times 30 min (again using the MSS) with 1 mL IP-wash buffer (PBS (20 mM NaH₂PO₄, 140 mM NaCl, pH 7.4), 0.1% BSA).
The activated dynabeads were resuspended with 1 mL PBS, 0.1% BSA, 0.02% Na-Azide; vortexed and centrifuged briefly.
The antibody activated dynabeads were stored at 4° C. until further use.

### B: Antigens Used for Immunoprecipitation:

1) Aβ(20-42) globulomer

The preparation of Aβ(20-42) globulomer is described in reference example 5.

2) Aβ(20-42) HMW oligomer (diluted dialysis)

The preparation of the Aβ(20-42) HMW oligomer is described in example 3.

### C: Immunoprecipitation (IP)

Aβ(20-42) globulomer, Aβ(20-42) HMW oligomer (diluted dialysis) samples were diluted with 20 mM NaH₂PO₄, 140 mM NaCl; 0.05% Tween 20, pH 7.4+0.1% BSA to a final concentration of 100 ng/ml.
25 μL of each antibody activated dynabeads of the following list were incubated with 0.1 mL of the diluted samples:
   8F5-Dynabeads
   5F7-Dynabeads
   4D10-Dynabeads
   7C6-Dynabeads
   IgG2b-Dynabeads
The immunoprecipitation was carried out by 1.5 h incubation under shaking at room temperature.
The dynabeads were immobilized with the MSS.
The supernatant was carefully removed and discarded.
The dynabeads were washed as follows:
   2 times 5 minutes with 500 μL 20 mM NaH₂PO₄, 140 mM NaCl, pH 7.4+0.1% BSA;
   1 time 3 minutes with 500 μL 2 mM NaH₂PO₄, 14 mM NaCl, pH 7.4;
   important: after the last removal of the washing buffer the reaction vials were centrifuged, placed back in the MSS and the remaining drops of fluid carefully removed;
   10 μL 50% CH₃CN, 0.5% TFA in H₂O were added to the reaction vial and vortexed;
   the reaction vials were incubated 10 minutes at RT under shaking;
   the dynabeads were immobilized with the MSS;
   the supernatant comprising the immunoprecipitated eluted Aβ species was carefully withdrawn (=IP-eluate).
      D: Surface-enhanced laser desorption ionization-mass spectrometry (SELDI-MS) quantification of the immunoprecipitated Aβ(20-42) peptide
1 μL IP-eluate was spotted onto a H4 Protein Chip Array (BioRad; Cat. no. C573-0028).
The spots were allowed to dry on a warm incubator plate.
CHCA-solution:
   5 mg CHCA were dissolved in 150 μL acetonitrile+150 μL 1% TFA=stock solution; stored at −20° C.
   Of the stock solution 10 μL were diluted with 20 μL acetonitrile and 20 μL 1% TFA=working CHCA-solution.
   2 μL of the working CHCA-solution was applied onto the spots
The spots were allowed to dry on a warm incubator plate and analyzed by SELDI-MS (Surface-Enhanced Laser Desorption Ionization-Mass Spectrometry; BioRad, Protein chip SELDI system enterprise edition)).
   Conditions: mass range: 0 to 10000 Da; focus mass: 4500 Da; matrix attenuation: 500 Da; sampling rate: 400 MHz; warming shots: 2 with energy: 1100 nj; data shots: 10 with energy 800-1000 nJoule; Partition 1 of 3.
   Analysis: the peak intensity of the respective Aβ(20-42) peptide mass peaks were quantified.

The results are shown in FIG. 9.

The Aβ globulomer specific antibodies 7C6, 5F7 and 4D10 (raised against the Aβ(20-42) truncated globulomer as immunogen) recognize Aβ(20-42) HMW oligomer (from example 3) and Aβ(20-42) truncated globulomer with qualitatively comparable affinities in an immunoprecipitation followed by relative SELDI-MS quantification of the Aβ(20-42) petide. The globulomer preferential antibody 8F5 (raised against the Aβ(1-42) globulomer as immunogen) does not detect the Aβ(20-42) HMW oligomer. This shows that under physiological buffer conditions of the immunoprecipitation the Aβ(20-42) HMW oligomer (example 3) presents the Aβ globulomer epitope. In contrast, immunization with a full length Aβ(1-42)-oligomer very likely results in antibodies (such as monoclonal antibody 8F5) which recognize a more N-terminal epitope Such N-terminal epitopes will not be generated with the Aβ(20-42) HMW oligomer as they are not present in the Aβ(20-42) HMW oligomer.

### Example 22

### Selectivity of Polyclonal Rabbit Immune Sera from Aβ(20-42) HMW Oligomer Immunization Against In Vitro Aβ(1-42) Monomer and Aβ Peptide in CSF of Alzheimer's Disease Patients

A: Activation of Dynabeads with Polyclonal Rabbit Anti-Sera
The stock-suspension of dynabeads (Dynabeads M-280 Sheep anti-Rabbit IgG, Invitrogen; Cat. no.: 112.04) was shaken carefully to prevent foaming.
1 mL was aseptically removed and transferred to a 1.5 mL reaction vial.
The dynabeads were washed 3 times 5 min with 1 mL immunoprecipitation (IP)-wash buffer (IP-wash-buffer: PBS (20 mM NaH₂PO₄, 140 mM NaCl, pH 7.4), 0.1% BSA). During the washing procedure the supernatant was carefully removed while the dynabeads were immobilized at the side of the reaction vial with a magnetic separator stand (MSS).
The washed dynabeads were incubated with 16 μl rabbit antisera in 1 mL PBS, 0.1% BSA.
The activation was carried out by overnight incubation under shaking at 4° C.
The activated dynabeads were washed 4 times 30 min (again using the MSS) with 1 mL IP-wash buffer (PBS (20 mM NaH₂PO₄, 140 mM NaCl, pH 7.4), 0.1% BSA).
The activated dynabeads were resuspended with 1 mL PBS, 0.1% BSA, 0.02% Na-Azide; vortexed and centrifuged briefly.
The antisera activated dynabeads were stored at 4° C. until further use.

### B: Antigens Used for Immunoprecipitation:

1. Aβ(1-42) monomer (NH₄OH)

The preparation of Aβ(1-42) (NH₄OH) is described in reference example 10.

2. Aβ(1-42) monomer (0.1% NaOH)

The preparation of Aβ(1-42) monomer (0.1% NaOH) is described in reference example 11.

3. Aβ(20-42) truncated globulomer

The preparation of Aβ(20-42) truncated globulomer (0.1% NaOH) is described in reference example 5.

4. Alzheimer's disease CSF

### C: Immunoprecipitation (IP)

Aβ(1-42) monomer (NH₄OH), Aβ(1-42) monomer(NaOH) and Aβ(20-42) globulomer were diluted with 20 mM NaH₂PO₄, 140 mM NaCl; 0.05% Tween 20, pH 7.4+0.1% BSA to a final concentration of 4 ng/ml and for each immunoprecipitation 50 μl of the Alzheimer's disease CSF samples were diluted with 200 μl 20 mM NaH₂PO₄, 140 mM NaCl; 0.05% Tween 20, pH 7.4 (without BSA)
25 μL of each antisera activated dynabeads of the following list were incubated with 0.5 mL of the diluted Aβ(1-42) monomer (NH₄OH), Aβ(1-42) monomer (NaOH) and Aβ(20-42) globulomer samples and with 250 μl of the diluted Alzheimer's disease CSF samples:
   Preimmune 2687-Dynabeads (Antiserum from rabbit #2687 before immunization with Aβ(20-42) HMW oligomer (from example 1))
   BA197-Dynabeads (Antiserum from rabbit #BA197 immunized with Aβ(1-42) globulomer (WO 2004/067561, Example 25d, Serum d1)
   5600-Dynabeads (Antiserum from rabbit #5600 immunized with Aβ(20-42) globulomer (WO 2004/067561, Example 25a, Serum a1)
   2687-Dynabeads (Antiserum from rabbit #2687 immunized with Aβ(20-42) HMW oligomer (from example 1))
The immunoprecipitation was carried out for 16 h over night incubation under shaking at 6° C.
The dynabeads were immobilized with the MSS.
The supernatant was carefully removed and discarded.
The dynabeads were washed as follows:
   2 times 5 minutes with 500 μL 20 mM NaH₂PO₄, 140 mM NaCl, pH 7.4+0.1% BSA;
   1 time 3 minutes with 500 μL 2 mM NaH₂PO₄, 14 mM NaCl, pH 7.4;
   after the last removal of the washing buffer the reaction vials were centrifuged, placed back in the MSS and the remaining drops of fluid carefully removed;
   8 μL 50% CH₃CN, 0.5% TFA in H₂O were added to the reaction vial and vortexed;
   the reaction vials were incubated 10 minutes at RT under shaking;
   the dynabeads were immobilized with the MSS;
   the supernatant comprising the immunoprecipitated eluted Aβ species was carefully withdrawn (=IP-eluate).

### D. Western Blot Analysis of Immunoprecipitated Samples:

### SDS-PAGE:

SDS-sample buffer:
   0.3 g SDS
   4 mL 1 M Tris/HCl pH 6.8
   8 mL glycerol
   70 μL1% bromphenolblue in ethanol
   add H₂O to 50 mL
Running buffer:
   7.5 g Tris
   36 g Glycine
   2.5 g SDS
   add H₂O to 2.5 L
SDS-PAGE gel system:
   18% Tris/Glycine Gel: (Invitrogen Inc., Cat. no.: EC65055BOX)
8 μL IP-eluate were added to 14 μL sample buffer (900 μL SDS-sample buffer+35 μL 1 M Tris-solution in H₂O+60 μL 85% glycerol). The resulting samples are loaded onto a 18% Tris/Glycin Gel (Invitrogen Inc., Cat. no.: EC65055BOX). The SDS-PAGE is conducted at a constant current of 20 mA.

### Western Blot Procedure:

Subsequent to electrophoresis, the gel was blotted for 45 minutes at 75 mA onto a nitrocellulose membrane (7.5 cm×9 cm, 0.2 μm, BioRad) using a semi-dry blotting chamber (BioRad).
Blot buffer:
   6 g Tris
   28.1 g glycine
   500 mL methanol
   add H₂O to 2.5 L

### Western Blot Immunostaining:

### Materials:

Anti-Aβ antibody 6E10 (Signet; Cat. No. 9320), 82E1 (IBL, #10323)
Anti-Mouse-POD (Jackson ImmunoResearch, Cat. no.: 715-035-150)
Detection reagent:
   Super Signal West Pico Substrate (Pierce, Cat. no.: 34077)
   Super Signal West Femto Maximum Sensitivity Substrate (Pierce, Cat. no.: 34096)
Bovine Serum Albumin (BSA, Serva, Cat. no.: 11926)
low fat milk powder (Lasana)
Blocking reagent:
   2% BSA in PBST
TBS:
   25 mM Tris/HCl
   150 mM NaCl buffer, pH 7.5
TTBS:
   25 mM Tris/HCl
   150 mM NaCl buffer
   0.05% Tween 20, pH 7.5
PBS:
   20 mM NaH₂PO₄ buffer
   140 mM NaCl buffer, pH 7.5
PBST:
   20 mM NaH₂PO₄ buffer
   140 mM NaCl buffer
   0.05% Tween 20, pH 7.5
Antibody solution I:
   1 μg/mL 6E10 in 20 mL 3% low fat milk in TBS (used for the detection of the in vitro Aβ forms Aβ(1-42) monomer (NH₄OH), Aβ(1-42) monomer (NaOH) and Aβ(20-42) globulomer
   0.2 μg/ml 82E1 in 20 mL 3% low fat milk in TBS (used for the detection of the Aβ peptide in Alzheimer's disease CSF)
Antibody solution II:
   1:10000 diluted anti-mouse-POD in 20 mL 3% low fat milk in TBS

### Procedure:

1) The Western blot was boiled for 10 minutes in PBS.
2) Blocking:
   The Western blot was incubated for 16 h at 6° C. with 50 mL blocking reagent.
3) Washing:
   The blocking solution was discarded and the Western blot washed with 50 mL TTBS for 10 minutes at room temperature.
   The blocking solution was discarded and the Western blot washed with 50 mL TBS for 10 minutes at room temperature.
4) Antibody solution I:
   The washing solution was discarded and the Western blot incubated with antibody solution I for 4 h at room temperature.
5) Washing:
   The blocking solution was discarded and the Western blot washed with 50 mL TTBS for 10 minutes at room temperature.
   The blocking solution was discarded and the Western blot washed with 50 mL TTBS for 10 minutes at room temperature.
   The blocking solution was discarded and the Western blot washed with 50 mL TBS for 10 minutes at room temperature.
6) Antibody solution II:
   The washing solution was discarded and the Western blot incubated with antibody solution II for 1 h at room temperature.
7) Washing:
   The blocking solution was discarded and the Western blot washed with 50 mL TTBS for 10 minutes at room temperature.
   The blocking solution was discarded and the Western blot washed with 50 mL TTBS for 10 minutes at room temperature.
   The blocking solution was discarded and the Western blot washed with 50 mL TBS for 10 minutes at room temperature.
8) Development and quantitative analysis:
   The washing solution was discarded.
   Two mL Super Signal West Pico Substrate Enhancer and 2 mL Peroxide Solution were mixed.
   The resulting 4 mL solution were added to the Western blot and the blot was incubated for 5 minutes in the dark.
   The blot was analyzed using a chemoluminescence imaging system (VersaDoc, BioRad). Five pictures were taken at 30, 97.5, 165, 232.5 and 300 seconds acquisition time.
   The picture at which no saturation of the trace (intensity x mm) of the Aβ protein bands occurred was quantitatively analyzed using the software package Quantity one, Version 4.5.0 (BioRad).
   After imaging the Super Signal West Pico solution was discarded and the Western blot washed with 50 mL TBS for 10 minutes at room temperature.
   The washing solution was discarded.
   Two mL Super Signal West Femto Maximum Sensitivity Substrate Enhancer and 2 mL Peroxide Solution were mixed.
   The resulting 4 mL solution were added to the Western blot and the blot was incubated for 5 minutes in the dark.
   The blot was analyzed using a chemoluminescence imaging system (VersaDoc, BioRad). Five pictures were taken at 30, 97.5, 165, 232.5 and 300 seconds acquisition time.
   The picture at which no saturation of the trace (intensity x mm) of the Aβ protein bands occurred was quantitatively analyzed using the software package Quantity one, Version 4.5.0 (BioRad).
   The calculated immunoprecipitated Aβ peptide of each individual used antigen or AD-CSF sample for the Preimmune 2687 antiserum was used as the unspecific background control and was substracted from all other individual used antigens or AD-CSF sample calculated immunoprecipitated Aβ peptide values

Results are shown in FIG. 10.

In FIG. 10A it is shown that a polyclonal antiserum from an immunization with Aβ(1-42) globulomer (antiserum BA197) recognizes in an immunoprecipitation Aβ monomers (Aβ(1-42) monomer (NaOH) and Aβ(1-42) monomer (NH₄OH)) and Aβ(20-42) globulomer, although to a lower extent. In contrast, polyclonal antiserum from immunization with the Aβ(20-42) HMW oligomer (antiserum 2687) selectively recognizes only the Aβ(20-42) globulomer. The latter is true also for polyclonal antiserum from Aβ(20-42) truncated globulomer immunization (antiserum 5600) but it shows a lower affinity for the Aβ(20-42) globulomer.

In FIG. 10B it is shown that a polyclonal antiserum from an immunization with Aβ(1-42) globulomer (antiserum BA197) recognizes in an immunoprecipitation Aβ peptide in CSF of Alzheimer's disease patients (CSF 55012, CSF 30028) and in healthy controls (CSF 7842). In contrast, polyclonal antiserum from immunization with the Aβ(20-42) HMW oligomer (antiserum 2687) does not recognize Aβ peptide in an immunoprecipitation in Alzheimer's disease patients (CSF 55012, CSF 30028) and in healthy controls (CSF 7842). The latter is true also for polyclonal antiserum from Aβ(20-42) truncated globulomer immunization (antiserum 5600).

### Example 23

### Selectivity of Polyclonal Rabbit Immune Sera from Aβ(20-42) HMW Oligomer Immunization Against in vitro Aβ(1-42) Monomer, Aβ(20-42) Globulomer, Aβ(20-42) HMW Oligomer

Sandwich-ELISA Protocol Used for the Determination of Aβ(20-42) globulomer recognition

### Reagent List:

1. F96 Cert. Maxisorp NUNC-Immuno Plate Cat.No.: 439454
2. Binding antibody
   Anti-Aβ mAb clone 7C6; conc.: 2.53 mg/ml OD 280 nm; stored at −80° C.
3. Coating-Buffer
   100 mM sodiumhydrogencarbonate; pH 9.6
4. Blocking Reagent for ELISA; Roche Diagnostics GmbH Cat. No.: 1112589
5. PBST-Buffer
   20 mM NaH₂PO₄; 140 mM NaCl; 0.05% Tween 20; pH 7.4
6. PBST+0.5% BSA-Buffer
   20 mM NaH₂PO₄; 140 mM NaCl; 0.05% Tween 20; pH 7.4+0.5% BSA; Serva cat.11926
7. Truncated Aβ(20-42) globulomer Standard:
   Conc.: 3.69 mg/ml amino acid analysis; stored at −80° C.
8. Primary rabbit sera:
   A) BA197 antiserum from rabbit immunized with Aβ(1-42) globulomer; WO 2004/067561 Example 25d, Serum d1;
   B) 5598 antiserum from rabbit immunized with Aβ(20-42) globulomer; WO 2004/067561 Example 25a, Serum a1;
   C) 5600 antiserum from rabbit immunized with Aβ(20-42) globulomer; WO 2004/067561 Example 25a, Serum a1;
   D) 2687-1 antiserum from rabbit immunized with Aβ(20-42) HMW oligomer (example 1) first bleeding;
   E) 2687-2 antiserum from rabbit immunized with Aβ(20-42) HMW oligomer (example 1); second bleeding after multiple immunization;
   F) 2687 preimmune serum prior to the immunization with Aβ(20-42) HMW oligomer (Example 1).
9. Label reagent:
anti-rabbit-POD conjugate; Jackson ImmunoResearch Ltd. Cat. No.: 111-036-045
10. Staining:
   TMB; Roche Diagnostics GmbH Cat. No.: 92817060;
   42 mM in DMSO
   3% H₂O₂ in water
   100 mM sodium acetate, pH 4.9
11. Stop Solution: 2M Sulfonic Acid
Method used in preparation of reagents:
1. Binding antibody:
   Anti-Aβ mAb clone 7C6 1:2530 to 1 μg/ml in coating buffer.
2. Blocking reagent:
   Blocking reagent was dissolved in 100 ml water to prepare the blocking stock solution and aliquots of 10 ml were stored at −20° C. 3 ml blocking stock solution was diluted with 27 ml water for each plate to block.
3. Aβ(20-42) globulomer standard dilution:
   A) 1 μl Aβ(20-42) globulomer standard stock solution (reference example 5) was added to 99 μl PBST+0.5% BSA=36.9 μg/ml.
   B) 13.5 μl Aβ(20-42) globulomer standard solution (36.9 μg/ml) was added to 4986 μl PBST+0.5% BSA=100 ng/ml.
Standard curve:

| No | Stock | | PBST + 0.5% BSA | | Final conc. | |
|---|---|---|---|---|---|---|
| 1 | 2 | ml B | 0 | ml | 100000 | pg/ml |
| 2 | 0.633 | ml (1) | 1.367 | ml | 31600 | pg/ml |
| 3 | 0.633 | ml (2) | 1.367 | ml | 10000 | pg/ml |
| 4 | 0.633 | ml (3) | 1.367 | ml | 3160 | pg/ml |
| 5 | 0.633 | ml (4) | 1.367 | ml | 1000 | pg/ml |
| 6 | 0.633 | ml (5 | 1.367 | ml | 316 | pg/ml |
| 7 | 0.633 | ml (6) | 1.367 | ml | 100 | pg/ml |
| 8 | 0 | ml | 2 | ml | 0.0 | pg/ml |

4. Primary rabbit sera:
   The rabbit sera were diluted in PBST+0.5% BSA buffer. The dilution factor was 1/2000. The sera were used immediately.
5. Label Reagent:
   Anti-rabbit-POD conjugate lyophilizate was reconstituted in 0.5 ml water. 500 μl glycerol was added and aliquots of 100 μl were stored at −20° C. for further use. The concentrated Label reagent was diluted in PBST-buffer. The dilution factor was 1/5000. The reagent was used immediately.
6. TMB solution:
   20 ml 100 mM of sodium acetate, pH 4.9, was mixed with 200 μl of the TMB solution and 29.5 μl 3% peroxide solution. The solution was used immediately.
Standard Plate Setup (Note that the standard for each serum was run in duplicate)

| | 1 | 2 | 3 | 4 | 5 | 6 | 7 | 8 | 9 | 10 | 11 | 12 |
|---|---|---|---|---|---|---|---|---|---|---|---|---|
| | sera BA197 | | sera 5598 | | sera 5600 | | sera 2687-1 | | sera 2687-2 | | sera 2687 pre | |
| A | 10000 | 10000 | 10000 | 10000 | 10000 | 10000 | 10000 | 10000 | 10000 | 10000 | 10000 | 10000 |
| B | 3160 | 3160 | 3160 | 3160 | 3160 | 3160 | 3160 | 3160 | 3160 | 3160 | 3160 | 3160 |
| C | 1000 | 1000 | 1000 | 1000 | 1000 | 1000 | 1000 | 1000 | 1000 | 1000 | 1000 | 1000 |
| D | 316 | 316 | 316 | 316 | 316 | 316 | 316 | 316 | 316 | 316 | 316 | 316 |
| E | 100 | 100 | 100 | 100 | 100 | 100 | 100 | 100 | 100 | 100 | 100 | 100 |
| F | 31.6 | 31.6 | 31.6 | 31.6 | 31.6 | 31.6 | 31.6 | 31.6 | 31.6 | 31.6 | 31.6 | 31.6 |
| G | 10 | 10 | 10 | 10 | 10 | 10 | 10 | 10 | 10 | 10 | 10 | 10 |
| H | 0.0 | 0.0 | 0.0 | 0.0 | 0.0 | 0.0 | 0.0 | 0.0 | 0.0 | 0.0 | 0.0 | 0.0 |

Procedure used:
1. 100 μl binding antibody solution per well were applied and incubated overnight at 4° C.
2. The antibody solution was discarded and the wells were washed three times with 250 μl PBST-buffer.
3. 265 μl block solution per well were added and incubated 2 h at room temperature.
4. The block solution was discarded and the wells were washed three times with 250 μl PBST-buffer.
5. After preparation of the standard curve, 100 μl per well of the standards were applied to the plate. The plate was incubated 2 h at room temperature and overnight at 4° C.
6. The standard solution was discarded and the wells were washed three times with 250 μl PBST-buffer.
7. 100 μl of rabbit sera solution per well were added and incubated 2 h at room temperature.
8. The rabbit sera solution was discarded and the wells were washed three times with 250 μl PBST-buffer.
9. 200 μl label solution per well were added and incubated 1 h at room temperature.
10. The label solution was discarded and the wells were washed three times with 250 μl PBST-buffer.
11. 100 μl of TMB solution were added to each well and incubated 5-15 min at room temperature.
12. Color staining was observed and 50 μl of the stop solution per well were applied.
13. The absorbance was read at 450 nm.
   Sandwich-ELISA protocol used for the determination of Aβ(20-42) HMW oligomer

### Reagent List:

1. F96 Cert. Maxisorp NUNC-Immuno Plate Cat. No.:439454
2. Binding antibody
   Anti-Aβ mAb clone 7C6; conc.: 2.53 mg/ml OD 280 nm; stored at −80° C.
3. Coating-Buffer
   100 mM sodium hydrogencarbonate; pH 9.6
4. Blocking Reagent for ELISA; Roche Diagnostics GmbH Cat. No.: 1112589
5. PBST-Buffer
   20 mM NaH₂PO₄; 140 mM NaCl; 0.05% Tween 20; pH 7.4
6. PBST+0.5% BSA-Buffer
   20 mM NaH₂PO₄; 140 mM NaCl; 0.05% Tween 20; pH 7.4+0.5% BSA; Serva cat.11926
7. Aβ(20-42) HMW oligomer standard:
   Aβ(20-42) HMW oligomer as described in example 3; conc.: 1,29 mg/ml BCA Protein Assay; stored at ±80° C.
8. Primary rabbit sera:
   a. BA197 antiserum from rabbit immunized with Aβ(1-42) globulomer; Patent WO 2004/067561 Example 25d, Serum d1
   b. 5598 antiserum from rabbit immunized with Aβ(20-42) globulomer; Patent WO 2004/067561 Example 25a, Serum a1
   c. 5600 antiserum from rabbit immunized with Aβ(20-42) globulomer; Patent WO 2004/067561 Example 25a, Serum a1
   d. 2687-1 antiserum from rabbit immunized with Aβ(20-42) HMW oligomer (example 1) first bleeding from Jan. 21, 2008;
   e. 2687-2 antiserum from rabbit immunized with Aβ(20-42) HMW oligomer (example 1); second bleeding after multiple immunization from Feb. 12, 2008
   f. 2687 preimmune serum prior to the immunization with Aβ20-42 HMW oligomer (example 1)
9. Label reagent:
   anti-rabbit-POD conjugate; Jackson ImmunoResearch Ltd. Cat. No.: 111-036-045
10. Staining:
   TMB; Roche Diagnostics GmbH Cat. No.: 92817060;
   42 mM in DMSO
   3% H₂O₂ in water
   100 mM sodium acetate, pH 4.9
11. Stop Solution:
   2 M Sulfonic Acid
Method used in preparation of reagents:
1. Binding antibody:
   Anti-Aβ mAb clone 7C6 was diluted 1:2530 to 1 μg/ml in coating buffer.
2. Blocking reagent:
   Blocking reagent was dissolved in 100 ml water to prepare the blocking stock solution and store aliquots of 10 ml at −20° C.
      3 ml blocking stock solution was diluted with 27 ml water for each plate to block.
   3. Aβ(20-42) monomer form, standard dilution:
      A) Add 1 μl of Aβ(20-42) HMW oligomer standard stock solution to 99 μl PBST+0.5% % BSA=12.9 μg/ml
      B) Add 38.8 μl of Aβ20-42 HMW oligomer standard solution (12.9 μg/ml) to 4961 μl PBST+0.5% BSA=100 ng/ml
         Standard curve:

| No | Stock | | PBST + 0.5% BSA | | Final conc | |
|---|---|---|---|---|---|---|
| 1 | 2 | ml B | 0 | ml | 100000 | pg/ml |
| 2 | 0.633 | ml (1) | 1.367 | ml | 31600 | pg/ml |
| 3 | 0.633 | ml (2) | 1.367 | ml | 10000 | pg/ml |
| 4 | 0.633 | ml (3) | 1.367 | ml | 3160 | pg/ml |
| 5 | 0.633 | ml (4) | 1.367 | ml | 1000 | pg/ml |
| 6 | 0.633 | ml (5 | 1.367 | ml | 316 | pg/ml |
| 7 | 0.633 | ml (6) | 1.367 | ml | 100 | pg/ml |
| 8 | 0 | ml | 2 | ml | 0.0 | pg/ml |

Procedure used:
1. Primary antibody:
   The rabbit sera were diluted in PBST+0.5% BSA buffer. The dilution factor was 1/2000. The preparation was used immediately.
2. Label Reagent:
   Anti-rabbit-POD conjugate lyophilizate was reconstituted in 0.5 ml water. 500 μl glycerol were added and aliquots of 100 μl were stored at −20° C. for further use.
   The concentrated Label reagent was diluted in PBST-buffer. The dilution factor was 1/5000. The reagent was used immediately.
3. TMB solution:
   20 ml of 100 mM sodium acetate, pH 4.9, were mixed with 200 μl of the TMB solution and 29.5 μl 3% peroxide solution. The solution was used immediately.
      Standard Plate Setup (Note that all standards were run in duplicate)

| | 1 | 2 | 3 | 4 | 5 | 6 | 7 | 8 | 9 | 10 | 11 | 12 |
|---|---|---|---|---|---|---|---|---|---|---|---|---|
| | sera BA197 | | sera 5598 | | sera 5600 | | sera 2687-1 | | sera 2687-2 | | sera 2687 pre | |
| A | 10000 | 10000 | 10000 | 10000 | 10000 | 10000 | 10000 | 10000 | 10000 | 10000 | 10000 | 10000 |
| B | 3160 | 3160 | 3160 | 3160 | 3160 | 3160 | 3160 | 3160 | 3160 | 3160 | 3160 | 3160 |
| C | 1000 | 1000 | 1000 | 1000 | 1000 | 1000 | 1000 | 1000 | 1000 | 1000 | 1000 | 1000 |
| D | 316 | 316 | 316 | 316 | 316 | 316 | 316 | 316 | 316 | 316 | 316 | 316 |
| E | 100 | 100 | 100 | 100 | 100 | 100 | 100 | 100 | 100 | 100 | 100 | 100 |
| F | 31.6 | 31.6 | 31.6 | 31.6 | 31.6 | 31.6 | 31.6 | 31.6 | 31.6 | 31.6 | 31.6 | 31.6 |
| G | 10 | 10 | 10 | 10 | 10 | 10 | 10 | 10 | 10 | 10 | 10 | 10 |
| H | 0.0 | 0.0 | 0.0 | 0.0 | 0.0 | 0.0 | 0.0 | 0.0 | 0.0 | 0.0 | 0.0 | 0.0 |

Procedure used:
1. 100 μl of binding antibody solution per well were applied and incubated overnight at 4° C.
2. The antibody solution was discarded and the wells were washed three times with 250 μl PBST-buffer.
3. 265 μl block solution per well were added and incubated 2 h at room temperature.
4. The block solution was discarded and the wells were washed three times with 250 μl PBST-buffer.
5. After preparation of the standard curve, 100 μl per well of the standards were applied to the plate. The plate was incubated 2 h at room temperature and overnight at 4° C.
6. The standard solution was discarded and the wells were washed three times with 250 μl PBST-buffer.
7. 100 μl rabbit sera solution per well were added and incubated 2 h at room temperature.
8. The antibody solution was discarded and the wells were washed three times with 250 μl PBST-buffer.
9. 200 μl label solution per well were added and incubated 1 h at room temperature.
10. The label solution was discarded and the wells were washed three times with 250 μl PBST-buffer.
11. 100 μl of TMB solution were added to each well and incubated 5-15 min at room temperature.
12. Color staining was observed and 50 μl of the stop solution per well were applied.
13. The absorbance was read at 450 nm.

Sandwich-ELISA protocol used for the determination of Aβ(1-42) monomer in 0.1% NH₄OH

Reagent List:
1. F96 Cert. Maxisorp NUNC-Immuno Plate Cat. No.: 439454
2. Binding antibody
   Anti-Aβ mAb clone 6E10; Signet Cat. No. 9320; conc.: 1 mg/ml OD 280 nm; stored at −20° C.
3. Coating-Buffer
   100 mM sodium hydrogencarbonate; pH 9.6
4. Blocking reagent for ELISA; Roche Diagnostics GmbH Cat. No.: 1112589
5. PBST-Buffer
   20 mM NaH₂PO₄; 140 mM NaCl; 0.05% Tween 20; pH 7.4
6. PBST+0.5% BSA-Buffer
   20 mM NaH₂PO₄; 140 mM NaCl; 0.05% Tween 20; pH 7.4+0.5% BSA; Serva Cat. 11926
7. Aβ(1-42) Standard:
   Aβ(1-42) solid powder; Bachem Cat. No.: H-1368; stored at ±20° C.
8. Primary antibody:
   a. BA197 antiserum from rabbit immunized with Aβ(1-42) globulomer; WO 2004/067561 Example 25d, Serum d1
   b. 5598 antiserum from rabbit immunized with Aβ(20-42) globulomer; WO 2004/067561 Example 25a, Serum a1
   c. 5600 antiserum from rabbit immunized with Aβ(20-42) globulomer; WO 2004/067561 Example 25a, Serum a1
   d. 2687-1 antiserum from rabbit immunized with Aβ(20-42) HMW oligomer (example 1) first bleeding from Jan. 21, 2008;
   e. 2687-2 antiserum from rabbit immunized with Aβ(20-42) HMW oligomer (example 1); second bleeding after multiple immunization from Feb. 12, 2008
   f. 2687 preimmune serum prior to the immunization with A1320-42 HMW oligomer (example 1).
9. Label reagent:
   anti-rabbit-POD conjugate; Jackson ImmunoResearch Ltd. Cat. No.: 111-036-045
10. Staining:
   TMB; Roche Diagnostics GmbH Cat. No.: 92817060;
   42 mM in DMSO
   3% H₂O₂ in water
   100 mM sodium acetate, pH 4.9
11. Stop Solution:
   2 M Sulfonic acid
Method used in preparation of reagents:
1. Binding antibody:
   Anti-Aβ mAb clone 6E10 was diluted 1:1000 to 1 μg/ml in coating buffer.
2. Blocking reagent:
   Blocking reagent was dissolved in 100 ml water to prepare the blocking stock solution and aliquots of 10 ml were stored at ±20° C.
   3 ml blocking stock solution was diluted with 27 ml water for each plate to block.
3. Aβ(1-42) monomer form, standard dilution:
   Aβ(1-42) monomer standard stock: 0.5 mg of Aβ(1-42) were dissolved in 250 μl 0.1% NH₄OH; (conc.: 2 mg/ml; freshly prepared) and used immediately.
   A) 10 μl of Aβ(1-42)-monomer standard stock solution were dissolved in 990 μl PBST+0.5% BSA=20 μg/ml.
   B) 25 μl of Aβ(1-42)-monomer standard stock solution (20 μg/ml) were added to 4975 μl PBST+0.5% BSA=100 ng/ml.
Standard curve:

| No | Stock | | PBST + 0.5% BSA | | Final conc. | |
|---|---|---|---|---|---|---|
| 1 | 2 | ml B | 0 | ml | 100000 | pg/ml |
| 2 | 0.633 | ml (1) | 1.367 | ml | 31600 | pg/ml |
| 3 | 0.633 | ml (2) | 1.367 | ml | 10000 | pg/ml |
| 4 | 0.633 | ml (3) | 1.367 | ml | 3160 | pg/ml |
| 5 | 0.633 | ml (4) | 1.367 | ml | 1000 | pg/ml |
| 6 | 0.633 | ml (5 | 1.367 | ml | 316 | pg/ml |
| 7 | 0.633 | ml (6) | 1.367 | ml | 100 | pg/ml |
| 8 | 0 | ml | 2 | ml | 0.0 | pg/ml |

4. Primary antibody:
   The rabbit sera were diluted in PBST+0.5% BSA buffer and used immediately. The dilution factor was 1/2000.
5. Label Reagent:
   Anti-rabbit-POD conjugate lyophilizate was reconstituted in 0.5 ml water. 500 μl glycerol were added and aliquots of 100 μl were stored at ±20° C. for further use.
   The concentrated label reagent was diluted in PBST-buffer. The dilution factor was 1/5000. The reagent was used immediately.
6. TMB solution:
   20 ml of 100 mM sodium acetate, pH 4.9, were mixed with 200 μl of the TMB solution and 29.5 μl 3% peroxide solution. The solution was used immediately.
Standard Plate Setup (Note that all Standards were Run in Duplicate)

| | 1 | 2 | 3 | 4 | 5 | 6 | 7 | 8 | 9 | 10 | 11 | 12 |
|---|---|---|---|---|---|---|---|---|---|---|---|---|
| | sera BA197 | | sera 5598 | | sera 5600 | | sera 2687-1 | | sera 2687-2 | | sera 2687 pre | |
| A | 10000 | 10000 | 10000 | 10000 | 10000 | 10000 | 10000 | 10000 | 10000 | 10000 | 10000 | 10000 |
| B | 3160 | 3160 | 3160 | 3160 | 3160 | 3160 | 3160 | 3160 | 3160 | 3160 | 3160 | 3160 |
| C | 1000 | 1000 | 1000 | 1000 | 1000 | 1000 | 1000 | 1000 | 1000 | 1000 | 1000 | 1000 |
| D | 316 | 316 | 316 | 316 | 316 | 316 | 316 | 316 | 316 | 316 | 316 | 316 |
| E | 100 | 100 | 100 | 100 | 100 | 100 | 100 | 100 | 100 | 100 | 100 | 100 |
| F | 31.6 | 31.6 | 31.6 | 31.6 | 31.6 | 31.6 | 31.6 | 31.6 | 31.6 | 31.6 | 31.6 | 31.6 |
| G | 10 | 10 | 10 | 10 | 10 | 10 | 10 | 10 | 10 | 10 | 10 | 10 |
| H | 0.0 | 0.0 | 0.0 | 0.0 | 0.0 | 0.0 | 0.0 | 0.0 | 0.0 | 0.0 | 0.0 | 0.0 |

### Procedure Used:

1. 100 μl of binding antibody solution per well were applied and incubated overnight at 4° C.
2. The antibody solution was discarded and the wells were washed three times with 250 μl PBST-buffer.
3. 265 μl block solution per well were added and incubated 2 h at room temperature.
4. The block solution was discarded and the wells were washed three times with 250 μl PBST-buffer.
5. After preparation of the standard curve, 100 μl per well of the standards were applied to the plate. The plate was incubated 2 h at room temperature and overnight at 4° C.
6. The standard solution was discarded and the wells were washed three times with 250 μl PBST-buffer.
7. 100 μl rabbit sera solution per well were added and incubated 2 h at room temperature.
8. The rabbit sera solution was discarded and the wells were washed three times with 250 μl PBST-buffer.
9. 200 μl label solution per well were added and incubated 1 h at room temperature.
10. The label solution was discarded and the wells were washed three times with 250 μl PBST-buffer.
11. 100 μl of TMB solution were added to each well and incubated 5-15 min at room temperature.
12. Color staining was observed and 50 μl of the stop solution per well were applied.
13. The absorbance was read at 450 nm.
   Results are shown in FIG. 11.

Antisera from active Aβ(20-42) HMW oligomer immunization (antiserum 2687-1) did not recognize Aβ(1-42) monomer (FIG. 11A) but Aβ(20-42) truncated globulomer (FIG. 11B) and Aβ(20-42) HMW oligomer (FIG. 11C) as shown by sandwich ELISA. This holds true even after multiple immunizations with Aβ(20-42) HMW oligomer (antiserum 2687-2). In contrast, antiserum from rabbits immunized with Aβ(1-42) globulomer (BA197) do not discriminate Aβ(1-42) monomer. Antiserum from rabbits immunized with Aβ(20-42) globulomer (5598 and 5600) discriminate Aβ(1-42) monomer but not as good as antisera from Aβ(20-42) HMW oligomer immunization (antiserum 2687-1 and antiserum 2687-2).

### Example 25

### Detection of Auto-Antibodies Against Aβ(20-42) HMW Oligomer in Human Plasma

### Materials:

Peptides and preparation of Aβ forms.

The Aβ(1-42) globulomer was prepared as described in reference example 3, whereas Aβ20-42 HMW oligomer (0.2% Tween 80) generation followed the description in example 9.

### Antibodies.

The 8F5 antibody is described in WO 2007/064972 (deposited with ATCC under designation number PTA-7238 on Dec. 1, 2005).

### Human Plasma, Csf, and Pbs-Extraction of Brain.

Plasma was provided from the German Red Cross (Mannheim, Germany). Upon arrival, the ˜300 mL plasma bags were thawed, aliquoted in 50 mL portions and kept at −20° C. CSF from healthy subjects was purchased from PrecisionMed (San Diego, Calif., USA). Small specimens of human AD brain were obtained from BrainNet (Munich, Germany). For PBS-extraction, 2700 μL ice-cold extraction buffer was added to 300 mg of cortex. Homogenization was performed by applying ten strokes with a glass potter (2 cm outer diameter, “S”-sized piston, Braun, Melsungen, Germany), followed by treatment with an ultra turrax (Janke & Kunkel, Staufen, Germany) for 30 s. After 30 min incubation on ice, the extracts were centrifuged for 20 min with 16.000 g at 20° C., and the supernatants (=PBS AD-brain extract) were kept at −80° C. until further use.

### Extraction Buffer:

### PBS (140 mM NaCl, 20 mM Na, H₂PO₄, pH 7.4)

Complete protease inhibitor (1 tablet used per 50 mL extraction buffer; Roche, Penzberg, Germany)

1 mM PMSF (freshly added)

Coupling of the Aβ forms to NHS-sepharose and preparation of the protein A/G columns. Coupling of either Aβ(1-42) globulomer or Aβ(20-42) HMW oligomer to NHS-sepharose 4FF (GE Healthcare, Munich, Germany) yielded the product Aβ(1-42) globulomer-sepharose or Aβ(20-42) HMW oligomer-sepharose, respectively. First, 4 mL NHS-sepharose was washed with ten bedvolumes (=40 mL) of ice-cold 1 mM HCl. Subsequently, the NHS-sepharose was washed with three to ten bedvolumes PBS until pH 7.4 was reached. Then, 1.8 mg of either Aβ(1-42) globulomer or Aβ(20-42) HMW oligomer was added to the NHS-sepharose, and the reaction was filled up with PBS to a final volume of 8 mL. Following 2 h incubation on a rotating wheel at RT, the reaction was continued over night at 4° C. Non-reacted NHS was quenched by washing three times with 10 mL of 0.5 M NaCl, 0.5 M ethanolamine, pH 8.3, and by 2 h incubation in 10 mL of 0.5 M NaCl, 0.5 M ethanolamine, pH 8.3 at RT. After washing three times with 10 mL PBS, the Aβ(1-42) globulomer-sepharose or respectively the Aβ(20-42) HMW oligomer-sepharose was stored as 50% slurry in PBS containing 0.02% NaN₃ at 4° C. The sepharose-only material was generated analogously without addition of any peptide or protein.

In order to isolate total immunoglobulin, equal volumes of protein A- and protein G-sepharose 4FF (GE Healthcare, Munich, Germany) were combined to yield the protein A/G-sepharose material.

For chromatography, ˜2 mL bedvolume of either the Aβ(1-42) globulomer-sepharose, Aβ(20-42) HMW oligomer-sepharose, sepharose-only or protein A/G-sepharose was used in combination with a Tricorn 10/20 column and an AKTA purifier system (both GE Healthcare, Munich, Germany).

### Analytical and Preparative Affinity Chromatography.

For affinity chromatography, 50 mL plasma aliquot was thawed and centrifuged for 10 min at 2570×g at RT. Loading of 45 mL of the supernatant onto either the Aβ(1-42) globulomer-sepharose, Aβ(20-42) HMW oligomer-sepharose, sepharose-only or protein A/G-sepharose column was performed by using a 50 mL Superloop (GE Healthcare, Munich, Germany) at 1 mL/min flow rate with PBS.

As controls for binding of globulomer specific antibodies to globulomer epitope present on the Aβ(20-42) HMW oligomer-sepharose or the Aβ(1-42) globulomer-sepharose, 50 μg each of m8F5 or 7C6 antibody were centrifuged for 10 min with 16100 g at 4° C., and loaded with a 500 μL sample loop under same conditions used for the plasma. For 8F5a binding to Aβ(1-42) globulomer-sepharose but not to Aβ(20-42) HMW oligomer-sepharose was expected, while for 7C6a binding to Aβ(20-42) HMW oligomer-sepharose but not to Aβ(1-42) globulomer-sepharose was expected.

Unbound proteins were removed by washing with PBS until the UV280 nm absorbance of the flow-through reached baseline (approximately after 15 mL).

### Chromatography Conditions:

1) linear high salt gradient starting from PBS to 100% 1.5 M NaCl in PBS within 10 mL
2) re-equilibration of the column to PBS within 10 mL
3) linear low pH gradient elution for 10 mL starting from 30% PBS, 70% 150 mM NaCl, 100 mM glycine, pH 2.8 to 50% PBS, 50% 150 mM NaCl, 100 mM glycine, pH 2.8.

For preparative purposes, the low pH elution was performed as step elution starting from PBS up to 50% PBS, 50% 150 mM NaCl, 100 mM glycine pH 2.8. 1 mL fractions were collected, immediately neutralized by addition of 15 μL 1.5 M Tris pH 8.8 in the sample collection vials prior to chromatography, and stored on ice until subsequent coupling of the antibodies to Dynabeads for immunoprecipitation purpose. Fractions eluting within the low pH region were combined and quantified by using the UV280 nm absorbance peak area.

### Preparation of Anti-Human Igg Dynabeads, Coupling of Antibodies to Dynabeads, and Immunoprecipitation.

### A: Generation of Anti-Human Igg Dynabeads

Anti-human IgG was purchased from Sigma-Aldrich (F_{c}- and F_{ab}-specific, 15260 and 12136, respectively, Seelze, Germany). Anti-human IgG Dynabeads were prepared as follows: 1 mL each, of 15260 and 12136 antibody, was combined, and dialysed against 100 mM borate pH 9.5 using a dialysis membrane with a cut-off of 12-14 kDa (5206010, Medicell, London, UK). 500 μL antibody dialysate was then added per 660 μL of borate-equilibrated Dynabeads (Tosyl-activated, #142.03, Invitrogen, Karlsruhe, Germany), incubated first for 10 min at 37° C. under shaking, and following an 500 μL addition of 0.5% BSA, borate for another 24 h. The beads were washed three times with PBS, and finally stored in 0.02% NaN₃, 0.1% BSA in PBS at 4° C. in a concentration of ˜7.10⁸ beads/mL.

B: Activation of Dynabeads with Monoclonal Mouse or Polyclonal Human Antibodies

Activation of Dynabeads with monoclonal mouse antibodies followed the description in example 21, part A. Activation of Dynabeads with polyclonal human antibodies was prepared in analogy to example 21, part A, with the exception that anti-human IgG Dynabeads as described in Part A of this example were used instead of anti-mouse IgG Dynabeads.

### C: Immunoprecipitation.

The immunoprecipitation was performed over night at 6° C. under shaking in 1 mL of 0.05% Tween 20 in PBS by either adding 50 μL CSF or 63 μL PBS-extract of AD brain per 25 μL antibody/Dynabead suspension resulting from part B. Following two washing steps with 0.1% BSA in 500 μL PBS and one with 500 μL 1/10 PBS, the beads were eluted by adding 20 μL SDS-PAGE non-reducing sample buffer.

### SDS-PAGE Sample Buffer (Non-Reducing):

0.3 g SDS
4 mL 1 M Tris/HCl pH 6.8
8 mL glycerol
100 μL of 1% bromphenolblue in ethanol (Fluka #18030) water added to 50 mL

### Dot- and Western-Blot Analysis

Dot blot analysis was performed in analogy to example 19, except that as antigens Aβ(1-42) globulomer (from reference example 3) or Aβ(20-42) HMW oligomer (0.2% Tween 80) (from example 9) were used in five 1/10 serial dilutions, each, starting from 100 μmol to 0.01 μmol. For Western blot detection of Aβ, first an SDS-PAGE was performed with a 4-20% Tris/Glycin gel (Invitrogen, Karlsruhe, Germany, EC60255BOX) in running buffer for 1 h at a constant current of 25 mA/gel using the Aβ(1-40; 1-42) standards as indicated. Subsequent to electrophoresis, the gels were blotted in blot buffer for 45 minutes at 75 mA/gel onto a PVDF membrane (7 cm×8.4 cm, 0.2 μm, BioRad, No.: 162-0174) using a semi-dry blotting chamber (BioRad). After transfer, the Western blot was boiled for 10 minutes in PBS, and blocked for 16 h at 6° C. with 50 mL blocking solution. The Western blot was washed with 25 mL TTBS+0.1% BSA three times for 10 minutes at room temperature, and incubated with antibody solution (biotinylated clone 82E1, purchased by IBL; cat number 10326; 1:500 diluted in 15 mL 3% low fat milk (Low fat milk powder (“Magermilchpulver”, JM Gabler Saliter, Obergünzburg, Germany) in TBS) for 2 h at room temperature. The Western blot was washed with 25 mL TTBS+0.1% BSA three times for 10 minutes at room temperature and incubated with ABC Reagent (ImmunoPure Standard Ultra-Sensitive ABC Peroxidase Staining Kit; Pierce, Cat. 32050; the reagent was allowed to react 30 min prior to incubation on the Western blot) for 1 h at room temperature. The Western blot was washed with 25 mL TTBS+0.1% BSA three times for 10 min at room temperature. For development of the blot, Super Signal West Femto Maximum Sensitivity Substrate was used (Pierce/Thermo Fisher Scientific, Cat. No. 34096). The blot was analyzed using a chemoluminescence imaging system (VersaDoc, BioRad). The picture at which no saturation of the trace (intensity x mm) of the Aβ peptide bands occurred was quantitatively analyzed using the software package Quantity one, Version 4.5.0 (BioRad). Aβ peptide amounts in the samples were calculated using the Aβ peptide standards on the Western blot.

### SDS-PAGE Running Buffer:

7.5 g Tris
36 g Glycine
2.5 g SDS
water added to 2.5 L

### Aβ(1-40; 1-42) Standards for Calibration/Quantitation:

Aβ(1-40); Bachem; cat. no. H-1194 was dissolved with 1 mg/ml in 50% acetonitril/0.5% trifluoroacetic acid
Aβ(1-42); Bachem; cat. no. H-1368 was dissolved with 1 mg/ml in 50% acetonitril/0.5% trifluoroacetic acid
10 μl of 1 mg/ml Aβ(1-40) and 10 μl of 1 mg/ml Aβ(1-42) were added to 9980 μl of 50% acetonitril/0.5% trifluoroacetic acid=2 μg/ml Aβ(1-40; 1-42)=2000 pg/μl

### Blot-Buffer:

6.18 g Boric Acid
H₂O added to 1.6 L
pH adjusted with 10 M NaOH to pH 9.0
400 mL methanol added

### ABC Reagent:

Prepared 30 min before use: 2 props Reagent A added to 10 mL TBS and mixed. 2 Drops Reagent B added and mixed again. 30 min at room temperature incubated prior to application on Western blot.

PBST: Stock solution 10×PBS (Invitrogen, Karlsruhe, Germany, #14200-067) diluted with H₂O to 1×PBS, 0.05% Tween 20 (Sigma-Aldrich, Steinheim, Germany, #P7949-500ML) added.

Blocking solution: 2% BSA in PBST (BSA: Serva, Cat. No.:11926)

TBS: 150 mM NaCl, 25 mM Tris, pH 7.5

TTBS+0.1% BSA: 150 mM NaCl, 25 mM Tris, 0.05% Tween 20, pH 7.5, 0.1% BSA (Blocker BSA in TBS10× Concentrate, Pierce, #37520)

The results are shown in FIGS. 16A, 16B, 17, 18A and 18B.

NHS(N-Hydroxy-succinimid)-sepharose based covalent linkage of Aβ(1-42) globulomer and Aβ(20-42) HMW oligomer did not disturb the 8F5 and 7C6 recognition of Aβ globulomer epitopes (8F5 can only detect Aβ(1-42) globulomer but not Aβ(20-42) HMW oligomer and 7C6 can only detect Aβ(20-42) HMW oligomer but not Aβ(1-42) globulomer). Analytical detection of Aβ(20-42) HMW oligomer-specific autoantibodies is in principle possible by UV214 nm chromatogram evaluation.

Aβ(20-42) HMW oligomer-specific autoantibodies could be isolated from human plasma (serum, IVIg most likely possible as well) even allowing subsequent immunoprecipitation (functional autoantibodies preparation). Aβ(20-42) HMW oligomer-based autoantibody preparation itself allowed the isolation of endogenous Aβ oligomer out of brain, which is not present in CSF. Thus, Aβ(20-42) HMW oligomer reflects an endogenous Aβ species. Aβ(20-42) HMW oligomer-specific autoantibodies show a 7C6 pattern in immunoprecipitation. 7C6 may therefore be similar to endogenously present autoantibodies. Aβ(20-42) HMW oligomer-based active immunisation might be an enhancement of an already existing B-cell immune response, rather than a creation of per se new antibodies.

### REFERENCE EXAMPLES

The following examples describe the synthesis of Aβ forms which do form part of the invention for reference purposes

### Reference Example 1

### Aβ(1-40) Monomer (0.1% NaOH)

1 mg Aβ(1-40) (Bachem Inc., cat. no. H-1194) was dissolved in 232.6 μl 0.1% NaOH in H₂O (freshly prepared) (=4.3 mg/ml=1 nmol/1 μl) and immediately shaken for 30 sec. at room temperature to get a clear solution. The sample was stored at −20° C. for further use.

### Reference Example 2

### Aβ(1-42) monomer (0.1% NaOH)

1 mg Aβ(1-42) (Bachem Inc., cat. no. H-1368) were dissolved in 222.2p1 0.1% NaOH in H₂O (freshly prepared) (=4.5 mg/ml=1 nmol/1 μl) and immediately shaken for 30 sec. at room temperature to get a clear solution. The sample was stored at −20° C. for further use.

### Reference Example 3

### Aβ(1-42) Globulomer

The Aβ(1-42) synthetic peptide (H-1368, Bachem, Bubendorf, Switzerland) was suspended in 100% 1,1,1,3,3,3-hexafluoro-2-propanol (HFIP) at 6 mg/mL and incubated for complete solubilization under shaking at 37° C. for 1.5 h. The HFIP acts as a hydrogen-bond breaker and is used to eliminate pre-existing structural inhomogeneities in the Aβ peptide. HFIP was removed by evaporation in a SpeedVac and Aβ(1-42) resuspended at a concentration of 5 mM in dimethylsulfoxide and sonicated for 20 s. The HFIP-pre-treated Aβ(1-42) was diluted in phosphate-buffered saline (PBS) (20 mM NaH₂PO₄, 140 mM NaCl, pH 7.4) to 400 μM and 1/10 volume 2% sodium dodecyl sulfate (SDS) (in H₂O) added (final concentration of 0.2% SDS). An incubation for 6 h at 37° C. resulted in the 16/20-kDa Aβ(1-42) globulomer intermediate. The 38/48-kDa Aβ(1-42) globulomer was generated by a further dilution with three volumes of H₂O and incubation for 18 h at 37° C. After centrifugation at 3000 g for 20 min the sample was concentrated by ultrafiltration (30-kDa cut-off), dialysed against 5 mM NaH₂PO₄, 35 mM NaCl, pH 7.4, centrifuged at 10,000 g for 10 min and the supernatant comprising the 38/48-kDa Aβ(1-42) globulomer withdrawn.

### Reference Example 4

### Aβ(12-42) Globulomer

2 ml of the Aβ(1-42) globulomer preparation of reference example 3 were admixed with 38 ml buffer (5 mM sodium phosphate, 35 mM sodium chloride, pH 7.4) and 150 μl of a 1 mg/ml GIuC endoproteinase (Roche) in water. The reaction mixture was stirred for 6 h at RT, and a further 150 μl of a 1 mg/ml GIuC endoproteinase (Roche) in water were subsequently added. The reaction mixture was stirred at RT for another 16 h, followed by addition of 8 μl of a 5 M DIFP solution. The reaction mixture was concentrated to approx. 1 ml via a 15 ml 30 kDa Centriprep tube. The concentrate was admixed with 9 ml of buffer (5 mM sodium phosphate, 35 mM sodium chloride, pH 7.4) and again concentrated to 1 ml. The concentrate was dialyzed at 6° C. against 1 l of buffer (5 mM sodium phosphate, 35 mM NaCl) in a dialysis tube for 16 h. The dialysate was adjusted to an SDS content of 0.1% with a 1% strength SDS solution in water. The sample was centrifuged at 10,000 g for 10 min and the Aβ(12-42) globulomer supernatant was withdrawn.

### Reference Example 5

### Aβ(20-42) Globulomer

1.59 ml of the Aβ(1-42) globulomer preparation of reference example 3 were admixed with 38 ml of buffer (50 mM MES/NaOH, pH 7.4) and 200 μl of a 1 mg/ml thermolysin solution (Roche) in water. The reaction mixture was stirred at RT for 20 h. Then 80 μl of a 100 mM EDTA solution, pH 7.4, in water were added and the mixture was furthermore adjusted to an SDS content of 0.01% with 400 μl of a 1% strength SDS solution. The reaction mixture was concentrated to approx. 1 ml via a 15 ml 30 kDa Centriprep tube. The concentrate was admixed with 9 ml of buffer (50 mM MES/NaOH, 0.02% SDS, pH 7.4) and again concentrated to 1 ml. The concentrate was dialyzed at 6° C. against 1 l of buffer (5 mM sodium phosphate, 35 mM NaCl) in a dialysis tube for 16 h. The dialysate was adjusted to an SDS content of 0.1% with a 2% strength SDS solution in water. The sample was centrifuged at 10,000 g for 10 min and the Aβ(20-42) globulomer supernatant was withdrawn.

### Reference Example 6

### Aβ Fibrils

1 mg Aβ(1-42) (Bachem Inc. Catalog Nr.: H-1368) were dissolved in 500 μl aqueous 0.1% NH₄OH (Eppendorff tube) and the sample was stirred for 1 min at room temperature. The sample was centrifuged for 5 min at 10,000×g and the supernatant was withdrawn. 100 μl of this freshly prepared Aβ(1-42) solution were neutralized with 300 μl 20 mM NaH₂PO₄; 140 mM NaCl, pH 7.4. The pH was adjusted to pH 7.4 with 1% HCl. The sample was incubated for 24 h at 37° C. and centrifuged (10 min at 10,000 g). The supernatant was discarded and the fibril pellet washed twice with 400 μl 20 mM NaH₂PO₄, 140 mM NaCl, pH 7.4 and then finally resuspended with 400 μl of 20 mM NaH₂PO₄; 140 mM NaCl, pH 7.4 by vortexing for 1 min.

### Reference Example 7

### sAPPα

Supplied from Sigma (cat. no. S9564; 25 μg in 20 mM NaH₂PO₄; 140 mM NaCl; pH 7.4). The sAPPa was diluted with 20 mM NaH₂PO₄, 140 mM NaCl, pH 7.4, 0.2 mg/ml BSA to 0.1 mg/ml (=1 μmol/p1).

### Reference Example 8

### Aβ(20-42) Monomer, 50% HFIP

2.9 mg of Aβ(20-42) solid powder was suspended in 145 μl 1,1,1,3,3,3-hexafluor-2-propanol and shaken for 1 h at 37° C. to get a clear solution with a concentration of 20 mg/ml. Then 1.1 μl of this solution were diluted with 99 μl of 50% HFIP in water. The Peptide concentration was 0.22 μg/μl=100 pmol/μl.

### Reference Example 9

### Aβ(22-42) Monomer, 50% HFIP

1 mg of Aβ(22-42) solid powder was suspended in 50 μl 1,1,1,3,3,3-hexafluor-2-propanol and shaken for 1 h at 37° C. to get a clear solution with a concentration of 20 mg/ml. Then 1 μl of this solution were diluted with 99 μl of 50% HFIP in water. The peptide concentration was 0.20 μg/μl=100 pmol/μl.

### Reference Example 10

### Aβ(1-42) Monomer (NH₄OH)

1 mg of Aβ(1-42) (Bachem Inc., cat. no. H-1368) was dissolved in 500 μl of 0.1% NH₄OH in H₂O (freshly prepared) and shaken for 30 seconds at room temperature to get a clear solution. The sample was stored at −20° C. for further use.

### Reference Example 11

### Aβ(1-42) Monomer (0.1% NaOH)

1 mg of Aβ(1-42) (Bachem Inc., cat. no.H-1368) was dissolved in 500 μl of 0.1% NaOH in H₂O (freshly prepared) and shaken for 30 seconds at room temperature to get a clear solution. The sample was stored at −20° C. for further use.

### Reference Example 12

### Size Exclusion Chromatography

Size exclusion chromatography parameters: SEC column: Superose 6 HR 10/300 GL (GE Health Care, catalogue no. 17-5172-01); flow-rate: 0.5 ml/min; detection of peptide with extinction at 215 nm; mobile phase: 20 mM NaH₂PO₄, 140 mM NaCl, 1% SDS, pH 7.4.

We claim:

1. An Aβ(X-38 . . . 43) oligomer, wherein X is selected from the group consisting of the numbers 12 . . . 24, characterized by a high molecular weight, or a derivative thereof.

2. The oligomer or derivative of claim 1, wherein X is 15, 18, 19, 20, 21, or 22.

3. The oligomer or derivative of claim 1, wherein Aβ(X-38 . . . 43) is Aβ(X-42).

4. The oligomer or derivative of claim 1, wherein Aβ(X-38 . . . 43) is Aβ(20-42).

5. The oligomer or derivative of claim 1, wherein the oligomer has an apparent molecular weight in size exclusion chromatography (1.0% SDS) of at least 200 kDa, at least 300 kDa, at least 400 kDa, or at least 500 kDa.

6. The oligomer or derivative of claim 1, wherein the oligomer is soluble.

7. The oligomer or derivative of claim 1, wherein the oligomer is reactive with a monoclonal antibody selected from the group consisting of 7C6, 7E5, 4D10 and 5F7.

8. The oligomer or derivative of claim 1, wherein the oligomer is capable of eliciting a polyclonal antiserum which comprises an antibody having a binding affinity to an oligomer or derivative of the invention or to an Aβ(20-42) truncated globulomer that is greater than the binding affinity of the antibody to at least one of Aβ form selected from the group consisting of monomeric Aβ(1-42), monomeric Aβ(1-40), monomeric Aβ(20-42), fibrillomeric Aβ(1-42), fibrillomeric Aβ(1-40) and Aβ(1-42) globulomer.

9. A process for preparing an Aβ(X-38 . . . 43) oligomer or a derivative thereof as defined in claim 1, which process comprises
(i) dissolving Aβ(X-38 . . . 43) peptide or a derivative thereof in a solvent; and
(ii) adding an amphipatic agent to the solution of Aβ(X-38 . . . 43) peptide or derivative and incubating the resulting mixture, or
which process comprises
(i) dissolving Aβ(X-38 . . . 43) peptide or a derivative thereof in a solvent; and
(ii) incubating the dissolved Aβ(X-38 . . . 43) peptide or derivative at a pH in the range of 8 to 10 and a temperature of more than 15° C.

10. The process of claim 9, wherein the solvent is a hydrogen bond-breaking agent such as HFIP or an aqueous solution of sodium hydroxide.

11. The process of claim 9, wherein the amphipatic agent is SDS, lauric acid, N-lauroylsarcosin, tert-octylphenol x 9-10EO, nonylphenol x 20EO, 3-(3-cholamidopropyldimethylammonio-1-propane sulfonate, dodecyl-N,N-dimethyl-3-amino-1-propane sulfonate, dodecylamine or a polyethoxylated sorbitol ester.

12. The process of claim 9, wherein the temperature of incubation is 15 to 50° C.

13. A process for preparing an Aβ(X-38 . . . 43) oligomer or a derivative thereof, wherein X is selected from the group consisting of the numbers 12 . . . 24, which process comprises
(i) dissolving monomeric Aβ(X-38 . . . 43) peptide or a derivative thereof in a hydrogen bond-breaking agent;
(ii) adding an amphipatic agent, mixing, optionally adjusting pH, pre-incubating, cenrifuging, obtaining the supernatant, incubating, centrifuging and obtaining the supernatant;
(iii) diluting, concentrating, centrifuging, and obtaining the supernatant; and
(iv) reducing salt concentration, centrifuging and obtaining the supernatant.

14. An Aβ(X-38 . . . 43) oligomer or a derivative thereof, wherein X is selected from the group consisting of the numbers 12 . . . 24, obtainable by a process as defined in claim 9.

15. A composition comprising an Aβ(X-38 . . . 43) oligomer or derivative as defined in claim 1, and optionally a pharmaceutically acceptable carrier.

16. The composition of claim 15 for treating or preventing amyloidosis.

17. Use of an Aβ(X-38 . . . 43) oligomer or derivative as defined in claim 1 for preparing a composition for diagnosing amyloidosis.

18. A method of identifying an agent capable of binding to an Aβ(X-38 . . . 43) oligomer or derivative as defined in claim 1, which method comprises the steps of: a) exposing one or more agents of interest to the oligomer or derivative for a time and under conditions sufficient for the one or more agents to bind to the oligomer or derivative; and b) identifying those agents which bind to the oligomer or derivative.

19. A method of enriching an agent capable of binding to an Aβ(X-38 . . . 43) oligomer or derivative as defined in claim 1 in a preparation comprising said agent, which method comprises the steps of: a) exposing to the Aβ(X-38 . . . 43) oligomer or derivative the preparation comprising the agent for a time and under conditions sufficient for the agent to bind to Aβ(X-38 . . . 43) oligomer or derivative; and b) obtaining the agent in enriched form.

20. A method of providing an antibody capable of binding to an Aβ(X-38 . . . 43) oligomer or derivative as defined in claim 1, which comprises
i) providing an antigen comprising the Aβ(X-38 . . . 43) oligomer or derivative;
ii) exposing an antibody repertoire to said antigen; and
iii) selecting from said repertoire an antibody which binds to the Aβ(X-38 . . . 43) oligomer or derivative.
